(19) United States
(12) Patent Application Publication
(10) Pub. No.: US 2025/0201341 A1
(43) Pub. Date: Jun. 19, 2025

(54) Methods and Systems for Identifying Disease-Specific Genetic Variants

(71) Applicant: William Gerard KEARNS, Poolesville, MD (US)

(72) Inventor: William Gerard KEARNS, Poolesville, MD (US)

(21) Appl. No.: 18/945,253

(22) Filed: Nov. 12, 2024

Related U.S. Application Data
(60) Provisional application No. 63/611,393, filed on Dec. 18, 2023.
(60) Provisional application No. 63/570,688, filed on Mar. 27, 2024.
(60) Provisional application No. 63/718,965, filed on Nov. 11, 2024.

(51) Int. Cl. G16B 20/20; G16B 25/10; G16B 40/30; G16H 10/60; G16H 20/10; G16H 50/30; G16H 50/70

(52) U.S. Cl. CPC G16B 20/20; G16B 25/10; G16B 40/30; G16H 10/60; G16H 20/10; G16H 50/30; G16H 50/70

(57) ABSTRACT

This application is directed to multiomic and biomimetic digital twin techniques for identifying disease-specific genetic variants. A computer system obtains information of subject genetic variants that are identified from a plurality of biological samples of a plurality of patients who are diagnosed with a target disease. A subset of subject genetic variants are selected based on a plurality of subject phenotypes of the target disease. The subset of subject genetic variants are ranked based on the plurality of subject phenotypes to generate subject genetic variant information. The computer system further obtains subject medical information of the plurality of patients. The computer system applies a biomimetic information model to process the subject genetic variant information, the subject medical information, and the general genetic variant information of the target disease and identify a set of target genetic variants associated with the phenotype of the disease(s) satisfying a variant selection criterion.

### RELATED APPLICATIONS

This application claims priority to U.S. Provisional Patent Application No. 63/611,393, titled “Application of Knowledge Engineering via the use of a Biomimetic Digital Twin Ecosystem, Phenotype Driven Variant Analysis, and Exome Sequencing to Understand the Molecular Mechanisms of Disease,” filed on Dec. 18, 2023, U.S. Provisional Patent Application No. 63/570,688, titled “Application of Knowledge Engineering via the use of a Biomimetic Digital Twin Ecosystem, Phenotype Driven Variant Analysis, and Exome Sequencing to Understand the Molecular Mechanisms of Disease,” filed on Mar. 27, 2024, and U.S. Provisional Patent Application No. 63/718,965, titled “Methods and Systems for Identifying Disease-Specific Genetic Variants,” filed on Nov. 11, 2024, which is hereby incorporated by reference in its entirety.

### TECHNICAL FIELD

The present application generally relates to biomimetic digital twin(s) technology including, but not limited to, methods and systems for analyzing biomedical information integrated from multiomics platforms and identifying biomarkers including disease-specific genetic variants for clinical diagnosis.

### BACKGROUND

Using data science, particularly artificial intelligence and machine learning, for clinical diagnosis of large biomedical data sets present several challenges. Data quality and heterogeneity pose significant issues, as biomedical data are often sourced from diverse platforms and in various formats, complicating integration and standardization efforts. Ensuring data privacy and security, while complying with strict regulations like HIPAA, adds another layer of complexity. The interpretability of machine learning models is also crucial, as clinicians need transparent, understandable outputs to trust and act on the model's predictions. Additionally, biases within the data can lead to inaccurate or unfair predictions, necessitating rigorous validation and ongoing monitoring of models. Finally, effectively incorporating these sophisticated tools into existing clinical workflows without disrupting routine practices requires careful planning, collaboration, and training among data scientists, healthcare providers, and IT professionals.

### SUMMARY

Disclosed embodiments include systems and methods for identifying and screening disease-specific genetic variants. Variants of unknown clinical significance (VUSs) are genetic variants that has been identified through genetic testing but whose significance to the function or health of an organism is not known. The present invention describes an innovative way to potentially re-classify VUSs. The disclosed system and methods combine multiomics, phenotype driven ranking analysis, biomedical models, and genotype-phenotype relationships, to identify VUSs closely associated with the phenotype of targeted diseases and hidden from traditional AI/ML/LLM approaches (“dark data”). The present invention allows one to understand the pathophysiology of studied disease, to correlate DNA or RNA variants with patient symptoms, to begin the process to reclassify VUSs as potential biomarkers and to reduce the time and cost of drug development.

In one aspect, a method for genetic testing (e.g., identifying disease-specific genetic variants) is implemented at a computer system. The method includes obtaining genome information of a plurality of subject genetic variants that are identified from a plurality of biological samples of a plurality of patients who are diagnosed with a target disease. A subset of subject genetic variants from the plurality of subject genetic variants are selected based on a plurality of subject phenotypes of the target disease. The subset of subject genetic variants are ranked based on the plurality of subject phenotypes to generate subject genetic variant information. The computer system further obtains subject medical information of the plurality of patients, including doctor-inputted description of the target disease collected from the plurality of patients and general genetic variant information, independently of the plurality of patients. The computer system applies a biomedical information model to process the subject genetic variant information, the subject medical information, and the general genetic variant information of the target disease and identify a set of target genetic variants associated with the phenotype of the disease(s) satisfying a variant selection criterion.

In some embodiments, the method further includes ranking the set of target genetic variants based on a correlation level with the plurality of subject phenotypes of the target disease, and in accordance with ranking, associating each of the set of target genetic variants with one of a plurality of predefined genetic significance levels.

In some embodiments, the method further includes assessing the set of target variants as therapeutic targets and determining one or more compounds of a drug configured to treat the target disease.

In another aspect, a method is implemented to identify a subject having a high risk of endometriosis, endometrial cancer, or an endometrial form of ovarian cancer. The method includes obtaining a biological sample from a subject, deriving a genomic DNA sample from the biological sample, and determining the said subject is a carrier of at least one single nucleotide polymorphism (SNP) or DNA variant within at least one gene selected from the group consisting of MUC 20, USP17L1, FAM66B, and DEFB109B.

In yet another aspect, a method is implemented to identify a subject having a high risk of rheumatoid arthritis. The method includes obtaining a biological sample from a subject, deriving a genomic DNA sample from the biological sample, and determining the said subject is a carrier of at least one single nucleotide polymorphism (SNP) or DNA variant within at least one gene selected from the group consisting of HIF1A, HLA-DOA, PTGER3, HIPK3, TGFBR3 and HIF1A-AS3.

In yet another aspect of the present application, a method comprises performing next generation sequencing (NGS) on patient and control samples. In some embodiments, sequencing by synthesis (SBS) is performed, as this is a widely adopted next-generation sequencing technology, as it detects single bases as they are incorporated into growing DNA strands with massively parallel capabilities. In some embodiments, specifically, a fluorescently labeled reversible terminator is imaged as each dNTP is added, and then is cleaved to allow incorporation of the next base. In some embodiments, since all four reversible terminator-bound dNTPs are present during each sequencing cycle, natural competition minimizes incorporation bias. In some embodiments, the result is true base-by-base sequencing that enables accurate data for a broad range of applications. In some embodiments, the method virtually eliminates errors and missed calls associated with strings of repeated nucleotides (homopolymers). In some embodiments, since the sequencing analysis is agnostic, other sequencing technologies such as Ion Torrent sequencing that is based on the principle of detecting hydrogen ions released when nucleotides are incorporated into a growing DNA template or Long-read sequencing (LRS) can be used. In some embodiments, LRS is a DNA sequencing technique that can read long strands of DNA at once, without breaking them into smaller fragments. In some embodiments, LRS is a type of next-generation sequencing (NGS) that can detect complex structural variants that are difficult to detect with short-read sequencing. In some embodiments, all types of DNA or RNA sequencing technologies can be used in the analysis. In some embodiments, the DNA variants include at least one SNP or DNA variant within at least one gene selected from the group consisting of MUC 20, USP17L1, FAM66B, and DEFB109B.

In yet another aspect of the present application, a method comprises performing next generation sequencing (NGS) on patient and control samples. In some embodiments, sequencing by synthesis (SBS) is performed, as this is a widely adopted next-generation sequencing technology, as it detects single bases as they are incorporated into growing DNA strands with massively parallel capabilities. In some embodiments, specifically, a fluorescently labeled reversible terminator is imaged as each dNTP is added, and then is cleaved to allow incorporation of the next base. In some embodiments, since all four reversible terminator-bound dNTPs are present during each sequencing cycle, natural competition minimizes incorporation bias. In some embodiments, the result is true base-by-base sequencing that enables accurate data for a broad range of applications. In some embodiments, the method virtually eliminates errors and missed calls associated with strings of repeated nucleotides (homopolymers). In some embodiments, since the sequencing analysis is agnostic, other sequencing technologies such as Ion Torrent sequencing that is based on the principle of detecting hydrogen ions release when nucleotides are incorporated into a growing DNA template or Long-read sequencing (LRS) can be used. In some embodiments, LRS is a DNA sequencing technique that can read long strands of DNA or RNA at once, without breaking them into smaller fragments. In some embodiments, LRS is a type of next-generation sequencing (NGS) that can detect complex structural variants that are difficult to detect with short-read sequencing. In some embodiments, all DNA or RNA sequencing technologies can be used in the analysis. In some embodiments, the DNA variants include at least one gene selected from the group consisting of HIF1A, HLA-DOA, PTGER3, HIPK3, TGFBR3 and HIF1A-AS3.

According to another aspect of the present application, a computer system includes one or more processing units, memory and a plurality of programs stored in the memory. The programs, when executed by the one or more processing units, cause the computer system to perform the method for identifying screening disease-specific genetic variants as described above.

According to another aspect of the present application, a non-transitory computer readable storage medium stores a plurality of programs for execution by a computer system having one or more processing units. The programs, when executed by the one or more processing units, cause the computer system to perform the method for identifying disease-specific genetic variants as described above as described above.

In yet another aspect of the present application, a kit includes at least one probe to selectively hybridizes to at least one nucleotide variance under high stringency conditions and amplifies the nucleotide variance sequence but does not amplify a corresponding wild-type sequence. The nucleotide variance includes at least one SNP within at least one gene selected from the group consisting of MUC 20, USP17L1, FAM66B, and DEFB109B.

In yet another aspect of the present application, a kit includes at least one probe to selectively hybridizes to at least one nucleotide variance under high stringency conditions and amplifies the nucleotide variance sequence but does not amplify a corresponding wild-type sequence, where the nucleotide variance includes at least one SNP within at least one gene selected from the group consisting of HIF1A, HLA-DOA, PTGER3, HIPK3, TGFBR3 and HIF1A-AS3.

Applied Artificial Intelligence (e.g., Large Language Models (LLMs) in biomedical research is accelerating, but effective discovery and validation requires a toolset without limitations or bias. In accordance with at least some embodiments disclosed herein is the realization that there is a need to advance the mathematical, statistical, and computational foundations of digital twins in applications across science, medicine, engineering, and society.

Artificial intelligence (AI), machine learning (ML), and large language models (LLM) has and continues to transform biomedical research and healthcare. The integration of these into biomedical research holds the promise of enhancing operational efficiency and reducing costs, improving diagnostic ability, uncovering new therapeutic targets, and enabling increasingly personalized medical treatments. Our healthcare future will be defined by how we leverage massive amounts of data for value and efficiency via AI/ML/LLM analysis.

While AI, ML and LLM holds tremendous promise for driving advances in biomedical research, these technologies, like all technologies, have limitations. Traditional AI/ML/LLM normalizes data and removes outliers, thus hindering the identification of hidden or “dark” data. Some ML techniques don't require normalization of data and the removal of outliers. In fairness, it must be said that this normalization of data to remove outliers can be argued is to simplify datasets and these can also be adjusted in other ways as well. AI, ML and LLMs also require a test training set to perform the analysis, and this could unintentionally introduce bias into the process. However, in using AI/ML/LLMs, one can modify and enhance training sets to identify the problem more accurately to be solved to reduce bias.

In accordance with at least some embodiments disclosed herein is the realization that the huge datasets required by traditional AI/ML/LLMs, and the associated scale of combinatorial math limit the ability of the algorithms to explore biological complexity, relegating most key relationships and critical interactions into dark data-data that is disconnected, unseen, unexplored and as a result, unanalyzed.

In accordance with at least some embodiments disclosed herein is the realization that there is a need to address these issues and to provide guidance to the biomedical community, e.g., using biomimetic digital twins' technology to more effectively model multidimensional and multi-scale biological complexity. “Digital twin”, “digital twins”, or “digital twin(s), interchangeably refers to a virtual replica of a physical object, process, or system that is used to simulate, monitor, optimize or predict the conditions of the object. “Biomimetic digital twins” refers digital twins that imitate biological processes in living organisms. Across multiple domains of science, engineering, and medicine, excitement is growing about the potential of digital twins to transform scientific research, industrial practices, and many aspects of daily life. A digital twin couples' computational models with a physical counterpart to create a system that is dynamically updated through bidirectional data flows as conditions change. Going beyond traditional simulation and modeling, digital twins could enable improved medical decision-making at the individual patient level, predictions of future weather and climate conditions over longer timescales, and safer, more efficient engineering processes.

The digital twin virtual representation (e.g., model types, fidelity, resolution, parameterization, and quantities of interest) may be chosen dynamically adapted to fit the decision task and computational constraints at hand, as well as acceptable cost. Due to the heterogeneity, complexity, multimodality, and breadth of biomedical data, the harmonization, aggregation, and assimilation of data and models to effectively combine these data into biomimetic digital twins is challenging. For many applications, the models that underlie the digital twin virtual representation must represent the behavior of the system across a wide range of spatial and temporal scales. For systems with a wide range of scales on which there are significant nonlinear scale interactions, it may be impossible to represent explicitly in a digital model the full richness of behavior at scales and including interactions. Technical challenges in modeling, computation, and data must be clarified to implementing digital twins for biomimetic use. Because medical data are often sparse and collecting data can be invasive to patients, researchers need strategies to create working models despite missing data.

A combination of data-driven and mechanistic models can be useful to this end, but these approaches can remain limited due to the complexities and lack of understanding of the full biological processes even when sufficient data are available. In addition, data heterogeneity and the difficulty of integrating disparate multimodal data, collected across different time and size scales, also engender significant research questions. New techniques are necessary to harmonize, aggregate, and assimilate heterogenous data for biomimetic digital twins. Furthermore, achieving interoperability and composability of models will be essential.

Taken collectively, the use of AI/ML/LLMs improves biomedical research but also faces gaps and some limits of AI methodologies when it comes to modeling and exploring the biological complexity of the real world. New theories and methods are required to address the multi-dimensional, multi-scale characteristics of problems in modeling and advanced analytics in general, and in biomedicine in particular. AI/ML/LLMs and digital twins could very well complement each other if the techniques are used most carefully and with enough knowledge in hands.

Across multiple domains of science, engineering, and medicine, excitement is growing about the potential of digital twins to transform scientific research, industrial practices, and many aspects of daily life. A digital twin combines computational models with a physical counterpart to create a system that is dynamically updated through bidirectional data flows as conditions change. Going beyond traditional simulation and modeling, digital twins could enable improved precision medicine and healthcare by more clearly understanding the pathophysiology of disease.

### BRIEF DESCRIPTION OF THE DRAWINGS

The accompanying drawings are included to provide a further understanding of the embodiments, are incorporated herein, and constitute a part of the specification. The drawings illustrate the described embodiments and together with the description serve to explain the underlying principles. Like reference numerals refer to corresponding parts.

FIG. 1 is an example data processing environment having one or more servers communicatively coupled to one or more client devices, in accordance with some embodiments.

FIG. 2A illustrates an example computer system configured to apply multiomics, phenotype-driven variant analysis, biomimetic digital twins, and genotype-phenotype relationships to obtain variants of unknown clinical significance (VUSs), in accordance with some embodiments.

FIG. 2B illustrates a series of experimental steps involved in identifying VUSs associated with pathophysiology of a targeted disease, in accordance with some embodiments.

FIG. 2C illustrates a series of omics-based tests to be combined for analyzing patient samples for obtaining subject variant information, in accordance with some embodiments.

FIG. 2D illustrates steps in obtaining subject variant information through the combination of DNA sequence analysis and differential gene expression transcriptomics analysis, in accordance with some embodiments.

FIG. 3 is a block diagram illustrating a computer system configured to process biomedical data, in accordance with some embodiments.

FIG. 4 is an example data processing environment for training and applying a neural network-based data processing model for processing visual and/or audio data, in accordance with some embodiments.

FIG. 5A is an example neural network applied to process content data in an NN-based data processing model, in accordance with some embodiments, and FIG. 5B is an example node in the neural network, in accordance with some embodiments.

FIG. 6 illustrates ranked variants based the correlations of variants with the phenotype of the targeted diseases obtained from tertiary analysis, in accordance with some embodiments.

FIG. 7A is a flowchart illustrating an exemplary process for identifying disease-specific genetic variants, in accordance with some embodiments. FIG. 7B is a continuation of flowchart FIG. 7A illustrating an exemplary process for identifying disease-specific genetic variants, in accordance with some embodiments.

FIG. 8 is a flowchart illustrating an exemplary process for identifying a subject having a high risk of endometriosis, endometrial cancer, or an endometrial form of ovarian cancer, in accordance with some embodiments.

FIG. 9 is a flowchart illustrating an exemplary process for identifying a subject having a high risk of rheumatoid arthritis, in accordance with some embodiments.

FIG. 10 illustrates a biomimetic digital twin ecosystem process tailored for analysis and user experience, in accordance with some embodiments.

FIG. 11 illustrates a biomimetic digital twin ecosystem including a qualitative metaontology with domains that can be populated and mapped independently by the subject matter experts, in accordance with some embodiments.

FIG. 12 illustrates a biomimetic digital twin ecosystem model and ecosystem design, in accordance with some embodiments.

FIG. 13 illustrates an engineering-level view of dark data discovery, in accordance with some embodiments.

### DETAILED DESCRIPTION

Reference will now be made in detail to specific embodiments, examples of which are illustrated in the accompanying drawings. In the following detailed description, numerous non-limiting specific details are set forth in order to assist in understanding the subject matter presented herein. It will be apparent to one of ordinary skill in the art that various alternatives may be used without departing from the scope of claims, and the subject matter may be practiced without these specific details. For example, it will be apparent to one of ordinary skill in the art that the subject matter presented herein can be implemented on many types of computer systems that support eye monitoring and diagnosis.

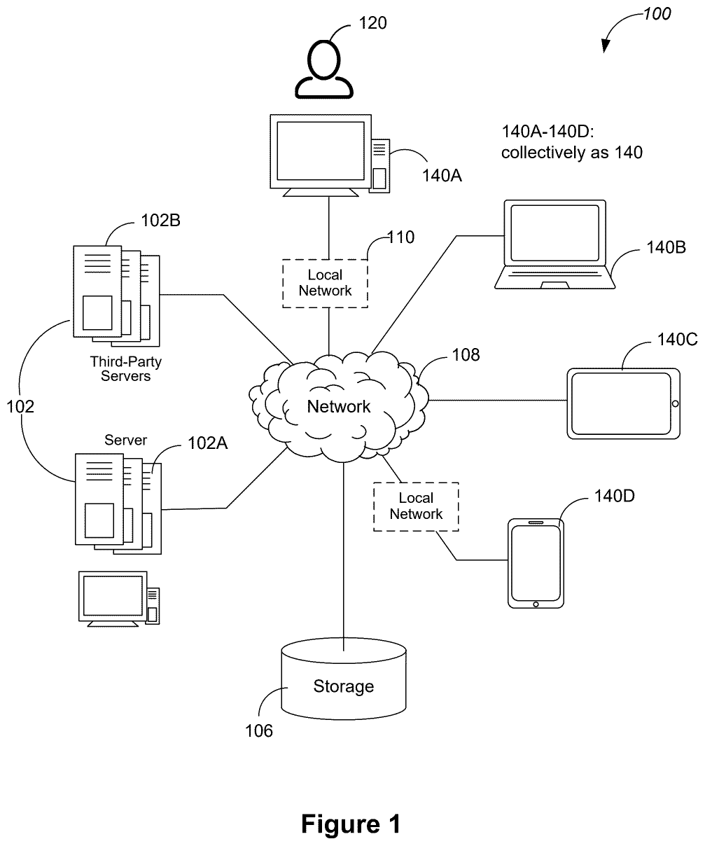

FIG. 1 is an example data processing environment 100 having one or more servers 102 communicatively coupled to one or more client devices 140, in accordance with some embodiments. The one or more client devices 140 may be, for example, desktop computers 140A, laptop computers 140B, tablet computers 140C, mobile phones 140D, or intelligent, multi-sensing, network-connected home devices (e.g., a surveillance camera 140E, a smart television device, a drone). Each client device 140 can collect data or user inputs, executes user applications, and present outputs on its user interface. The collected data or user inputs can be processed locally at the client device 140 and/or remotely by the server(s) 102. The one or more servers 102 provide system data (e.g., boot files, operating system images, and user applications) to the client devices 140, and in some embodiments, processes the data and user inputs received from the client device(s) 140 when the user applications are executed on the client devices 140. Examples of the user applications include, but are not limited to, a biomimetic digital twin application, knowledge graphs application, a medical history application, a sequence analysis application, a genetic variant interpretation application, a genetic variant ranking application, a genetic variant database search application. In some embodiments, the data processing environment 100 further includes a storage 106 for storing data related to the servers 102, client devices 140, and applications executed on the client devices 140.

The one or more servers 102 are configured to enable real-time data communication with the client devices 140 that are remote from each other or from the one or more servers 102. Further, in some embodiments, the one or more servers 102 are configured to implement data processing tasks that cannot be or are preferably not completed locally by the client devices 140. In some embodiments, the one or more servers 102 include a first server 106A for obtain subject genetic variant information, subject medical information, and general genetic variant information of a target disease, e.g., from one or more second servers 106B. The first server 106A is configured to apply a biomimetic information model to process the subject genetic variant information, the subject medical information, and the general genetic variant information of the target disease and identify a set of target genetic variants associated with a target disease.

The one or more servers 102, one or more client devices 140, and storage 106 are communicatively coupled to each other via one or more communication networks 108, which are the medium used to provide communications links between these devices and computers connected together within the data processing environment 100. The one or more communication networks 108 may include connections, such as wire, wireless communication links, or fiber optic cables. Examples of the one or more communication networks 108 include local area networks (LAN), wide area networks (WAN) such as the Internet, or a combination thereof. The one or more communication networks 108 are, optionally, implemented using any known network protocol, including various wired or wireless protocols, such as Ethernet, Universal Serial Bus (USB), FIREWIRE, Long Term Evolution (LTE), Global System for Mobile Communications (GSM), Enhanced Data GSM Environment (EDGE), code division multiple access (CDMA), time division multiple access (TDMA), Bluetooth, Wi-Fi, voice over Internet Protocol (VoIP), Wi-MAX, or any other suitable communication protocol. A connection to the one or more communication networks 108 may be established either directly (e.g., using 4G/4G connectivity to a wireless carrier), or through a network interface 110 (e.g., a router, switch, gateway, hub, or an intelligent, dedicated whole-home control node), or through any combination thereof. As such, the one or more communication networks 108 can represent the Internet of a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other computer systems that route data and messages.

In some embodiments, deep learning techniques are applied in the data processing environment 100 to process biomedical data obtained by an application executed at a client device 140 to identify information contained in the data, match the data with other data, categorize the biomedical data, or synthesize related data. In these deep learning techniques, data processing models are created based on one or more neural networks to process the data. These data processing models are trained with training data before they are applied to process the biomedical data. Subsequently to model training, the client device 140 obtains the biomedical data and processes the data using the data processing models locally.

In some embodiments, both model training and data processing are implemented locally at each individual client device 140. The client device 140 obtains the training data from the one or more servers 102 or storage 106 and applies the training data to train the data processing models.

Alternatively, in some embodiments, both model training and data processing are implemented remotely at a server 102 (e.g., the server 102A) associated with a client device 140. The server 102A obtains the training data from itself, a server 102B, or the storage 106 applies the training data to train the data processing models. The client device 140 obtains the biomedical data, sends the biomedical data to the server 102A (e.g., in an application) for data processing using the trained data processing models, receives data processing results from the server 102A, presents the results on a user interface (e.g., associated with the application) or implements some other functions based on the results. The client device 140 itself implements no or little data processing on the biomedical data prior to sending them to the server 102A.

Additionally, in some embodiments, data processing is implemented locally at a client device 140, while model training is implemented remotely at a server 102 (e.g., the server 102A) associated with the client device 140. The server 102A obtains the training data from itself, another server 102B, or the storage 106 and applies the training data to train the data processing models. The trained data processing models are optionally stored in the server 102A or storage 106. The client device 140 imports the trained data processing models from the server 102A or storage 106, processes the biomedical data using the data processing models, and generates data processing results to be presented on a user interface or used to initiate some functions locally.

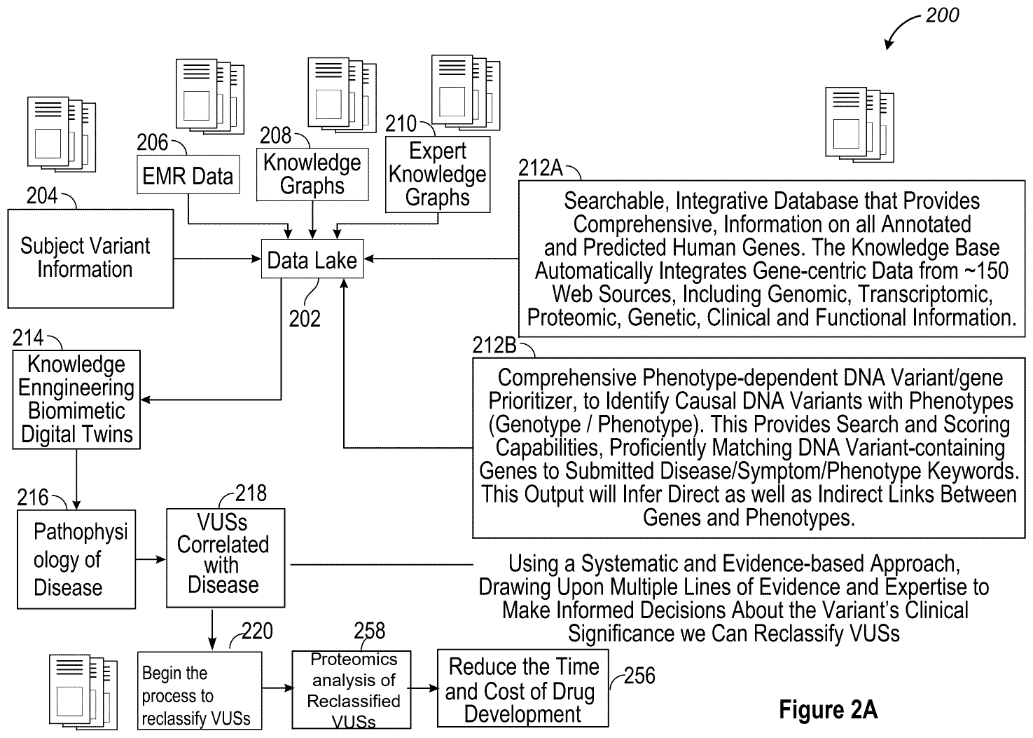

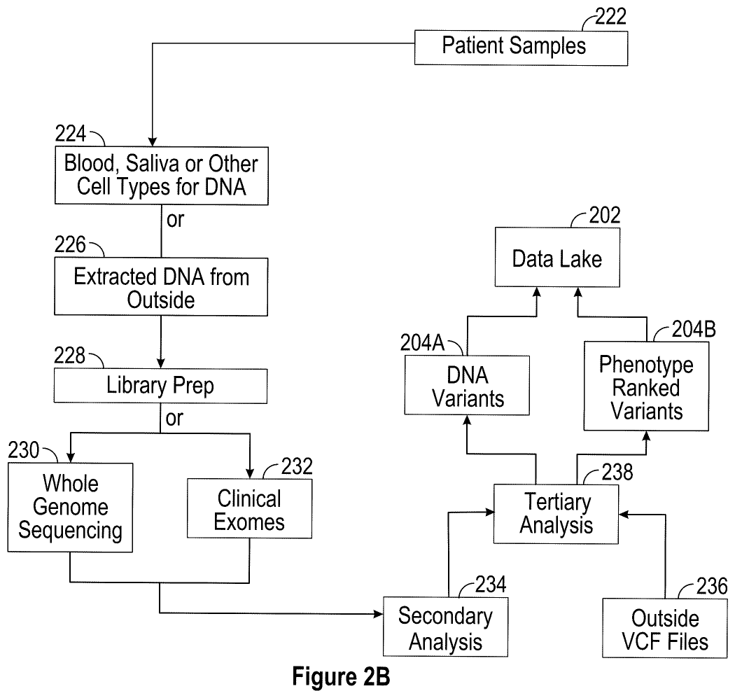

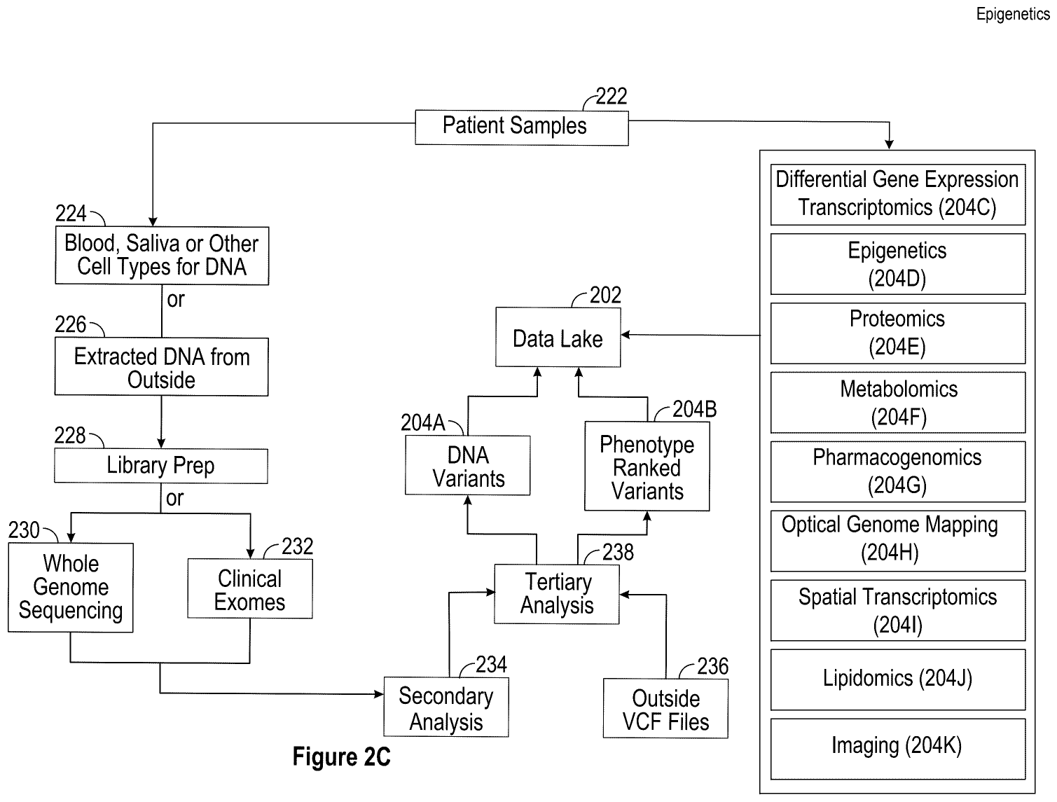

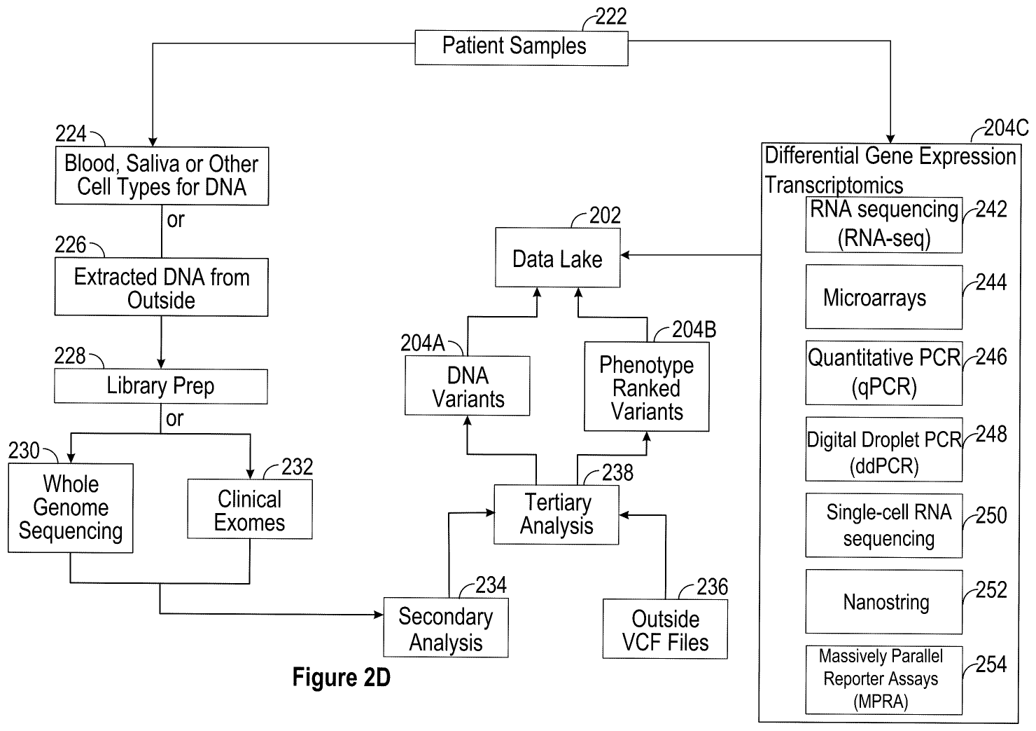

More specifically, in some implementations of this application, a computer system 200 includes a server 102A. The server 102A obtains genome information of a plurality of subject genetic variants that are identified from a plurality of biological samples of a plurality of patients who are diagnosed with a target disease, selects a subset of subject genetic variants from the plurality of subject genetic variants based on a plurality of subject phenotypes of the target disease, and ranks the subset of subject genetic variants based on the plurality of subject phenotypes to generate subject genetic variant information. The server 102 further obtains (e.g., one or more servers 102B) subject medical information of the plurality of patients, including doctor-inputted description of the target disease collected from the plurality of patients. The server 102 obtains (e.g., one or more servers 102B) general genetic variant information, independently of the plurality of patients. A biomimetic information model may be applied to process the subject genetic variant information, the subject medical information, and the general genetic variant information of the target disease and identify a set of target genetic variants from the subset of subject genetic variants, the set of target genetic variants satisfying a variant selection criterion. More details about the biomedical data being obtained by client device 140 and data processing results from the server 102 are discussed below with reference to FIGS. 2A and 2B.

FIG. 2A illustrates an example computer system 200 configured to apply multiomics, phenotype-driven variant analysis, biomimetic digital twins, and genotype-phenotype relationships to identify variants of unknown clinical significance (VUSs), in accordance with some embodiments. In some embodiments, a data lake 202 is created by obtaining genetic information of a plurality of subject genetic variants that are identified from a plurality of biological samples of a plurality of patients who have diagnosed with a targeted disease and genetic variant information independent of the patients. In some embodiments, the data lake 202 is imported with subject variant information 204. In some embodiments, the data lake 202 is imported with the subject medical information includes, but is not limited to, patient medical record, EMR data 206, family history of targeted diseases, and medical information of the plurality of patients. In some embodiments, the data lake 202 is imported with a first knowledge graph, e.g., a general knowledge graph 208 derived from research databases, such as PubMed, ClinVar, The Human Gene Mutation Database (HGMD), etc and a second expert knowledge graph, e.g., a private database, designed by experts in the disease studied 210. In some embodiments, the data lake 202 is imported with genetic variant information independent of the patients through biomedical databases. In some embodiments, at least two databases are utilized to obtain general genetic variant information independent of the patients. In some embodiments, a first database 212A is a searchable, integrative database providing information on annotated and predicted human genes. The first database 212A will automatically integrate gene-centric data from approximately 300 web sources, including genomic, transcriptomic, proteomic, genetic, clinical, and functional information. In some embodiments, a second database 212B is for obtaining general genetic variant information about associations between human gene variants and phenotypes through obtaining, from a second public database. In some embodiments, the second database 212B is phenotype-dependent DNA variant/gene prioritizer, to identify causal DNA variants with phenotypes (genotype/phenotype). The second database 212B provides search and scoring capabilities, proficiently matching DNA variant-containing genes to submitted disease/symptom/phenotype keywords. This output will infer direct as well as indirect links between genes and phenotypes.

In some embodiments, data from data lake 202 is exported into the biomimetic digital twin engine 214 for analysis. In some embodiments, the biomimetic digital twin engine 214 identified the pathophysiology of Disease 216 and produced a list of gene variants classified as VUSs potentially associated with pathophysiology of the targeted disease 218, where VUSs are assigned a genotype-phenotype ranking. In some embodiments, VUSs are ranked according to the number of times that they are present in patient samples 222 but absent from controls. In some embodiments, by using a systematic and evidence-based approach, drawing upon multiple lines of evidence and expertise to make informed decisions about the variant's clinical significance, the VUS can be reclassified. In some embodiments, the list of VUS 218 includes the genome information of the plurality of subject genetic variants includes information of an outlier genetic variant, e.g., corresponding to the set of target genetic variants. In some embodiment, those information of the plurality of subject genetic variants is used to begin to reclassify these variants as pathogenic. The information of the reclassified outlier VUS 220 is not included in the general genetic variant information, and the reclassified outlier VUS 220 is preserved in the set of target genetic variants after the biomedical information model is applied and applied to identify the set of target genetic variants.

In some embodiments, reclassified outlier VUS 220 can be obtained from Biomimetic Digital Twins engine 214 based on the obtained correlation level with the plurality of subject phenotypes of the target disease. In some embodiments, reclassifying outlier VUSs to one of a set of classes (pathogenic, likely pathogenic, uncertain significance, likely benign, and benign) reduces the time and cost of drug development 256.

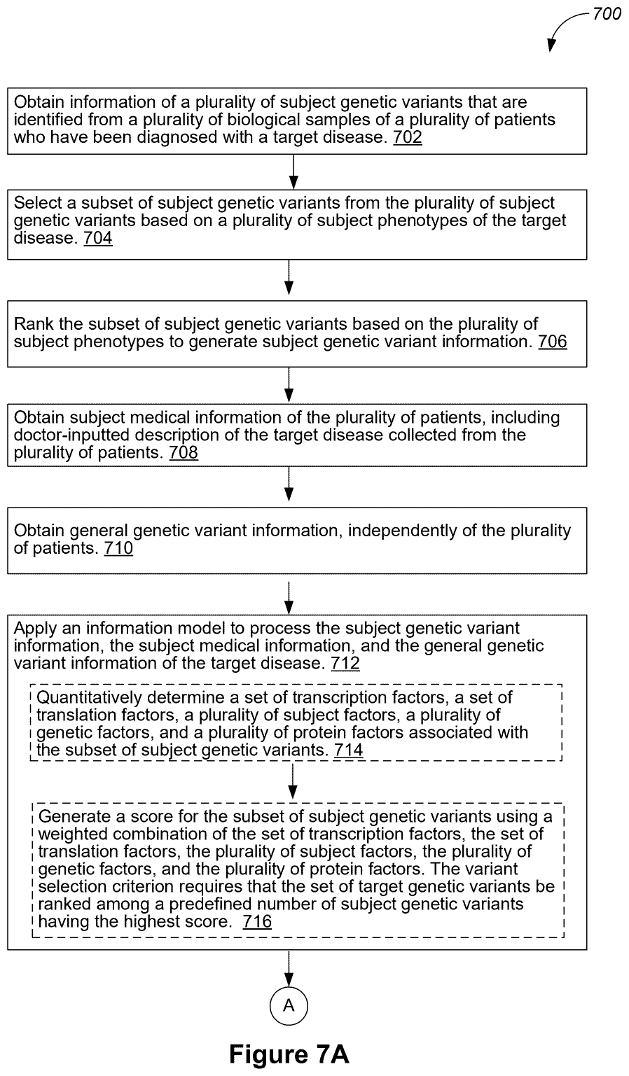

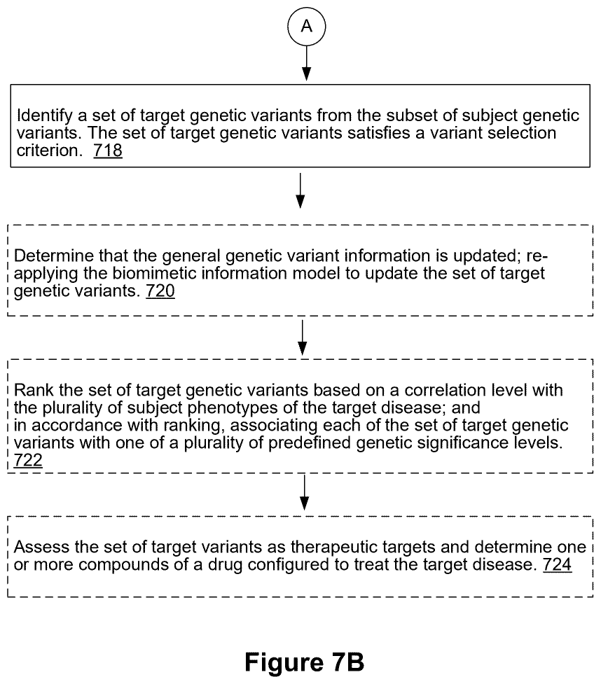

FIG. 2B illustrates a series of experimental steps involved in identifying VUSs associated with pathophysiology of a targeted disease, in accordance with some embodiments. In some embodiments, patient samples 222 are taken in a clinical diagnostic setting. In various embodiments, patient samples 222 include blood, saliva, or other cell types for DNA 224 or a combination thereof. Genetic information from the extracted DNA 226 of the patients is obtained. In some embodiments, a sequencing library 228 is constructed based on extracted DNA 226 obtained from patient samples 222. In some embodiments, whole exome or whole genome sequencing 230 is performed on each sample to determine the presence or absence of known pathogenic mutations, and variants of unknown clinical significance (VUS) associated with targeted diseases. In some embodiments, whole exome sequencing or whole genome sequencing 232 ordered by physicians with a signed informed consent are being analyzed. In some embodiments, secondary analysis 234 is performed on the sequencing data obtained from, e.g., whole genome sequencing or whole exome sequencing, where the secondary analysis compares a reference genome to the patient samples for generating genome information of a plurality of subject genetic variants identified from the plurality of patients associated with the targeted disease. In some embodiments, to obtain a subset of subject genetic variants, tertiary analysis 238 is performed on the results obtained from secondary analysis, optionally in combination with the any outside variant call format (VCF) or other files 236 about the genome information of variants from the patients. In some embodiments, tertiary analysis interprets the variant information based on knowledge about symptoms, phenotypes and gene-disease associations, from biomedical databases, medical guidelines and a wide variety of different bibliography content sources that are clinically relevant. In some embodiments, a filter 638 is applied to select the subset of subject genetic variants from the plurality of subject genetic variants based on one or more of: a confidence score, a population frequency of occurrence, a predicted deleterious level, or a biological impact. In some embodiments, genetic information about DNA variants 204A and genetic information about phenotype ranked variants 204B are downloaded into data lake 202. More details on different data sources 204-220 coupled to a data lake 202 and associated data operations 222-254 are discussed below with reference to FIGS. 7A and 7B.

FIG. 2C illustrates a series of omics-based tests involved analyzing patient samples 222 for obtaining datasets derived from biological samples, in accordance with some embodiments. In some embodiments, datasets obtained from one or more additional omics analysis can be added to the data lake 202. In some embodiments, the one or more additional omics analysis includes, but are not limited to, differential gene expression transcriptomics 204C, epigenetics 204D, proteomics 204E, metabolomics 204F, pharmacogenomics 204G, optical genome mapping (OGM) 204H, spatial transcriptomics 204I, lipidomics 204J, and/or biological imaging 204K.

In some embodiments, the transcriptomics 204C is the study of the complete set of RNA transcripts (the transcriptome) produced by the genome under specific circumstances or in a particular cell or tissue type. In some embodiments, the transcriptomics 204C provides a snapshot of gene expression at a given time, offering a dynamic view of how genes are regulated in response to various biological processes, environmental stimuli, or diseases. In some embodiments, the differential gene expression refers to the process of comparing the levels of gene expression between two or more biological conditions (e.g., healthy vs. diseased tissue, treated vs. untreated cells, different time points). In some embodiments, this analysis helps to identify genes that are upregulated (expressed at higher levels) or downregulated (expressed at lower levels) under specific conditions, providing insights into how genes respond to external stimuli, diseases, or developmental stages. In some embodiments, these analysis can also be performed on single cells (sc-RNA).

In some embodiments, the epigenetics 204D refers to the study of heritable changes in gene expression or cellular function that do not involve alterations in the underlying DNA sequence. In some embodiments, these changes are caused by chemical modifications to DNA or histone proteins, which regulate how genes are turned on or off. Epigenetic modifications can be influenced by environmental factors, lifestyle, and developmental stages, and they can be passed from one generation to the next or occur during an organism's lifetime.

In some embodiments, the proteomics 204E is the large-scale study of the entire set of proteins (the proteome) produced or modified by an organism, cell, or tissue under specific conditions. Proteins are the functional molecules responsible for carrying out most biological processes, and they can undergo various modifications and interactions that influence their function, structure, and activity. In some embodiments, proteomics aims to understand the structure, function, expression, and interactions of proteins in biological systems.

In some embodiments, the metabolomics 204F is the large-scale study of small molecules, commonly known as metabolites, within cells, tissues, or organisms. In some embodiments, these metabolites are the end products of various biochemical reactions in metabolism and reflect the physiological state of a cell or tissue at a given time. In some embodiments, by analyzing the complete set of metabolites (the metabolome), metabolomics provides a snapshot of cellular processes and offers insights into how biochemical pathways are functioning under specific conditions, such as health, disease, or environmental changes.

In some embodiments, the pharmacogenomics 204G is the study of how an individual's genetic makeup influences their response to drugs. In some embodiments, the pharmacogenomics 204G combines principles from pharmacology (the study of drugs and their effects) and genomics (the study of genes and their functions) to understand how genetic variations affect drug efficacy, safety, and metabolism. In some embodiments, the ultimate goal of pharmacogenomics is to enable personalized medicine, where drug treatments can be tailored to an individual's genetic profile for optimal effectiveness and minimal side effects.

In some embodiments, the optical genome mapping (OGM) 204H is a technique used to visualize and analyze the structure of large, complex genomes at high resolution. In some embodiments, the optical genome mapping (OGM) 204H provides a physical map of the genome by imaging long DNA molecules in their native state, enabling the detection of structural variations (SVs) such as large insertions, deletions, inversions, translocations, and duplications, which are difficult to identify with traditional sequencing methods.

In some embodiments, the spatial transcriptomics 204I is a technique used to map gene expression within the spatial context of a tissue or organism. Unlike traditional transcriptomics, which provides gene expression data without information about the physical location of cells, spatial transcriptomics preserves the spatial arrangement of cells while sequencing their RNA. In some embodiments, this technique allows researchers to understand how gene expression varies across different regions of a tissue, providing insights into tissue organization, cell-to-cell interactions, and how spatial gene expression patterns contribute to biological processes and diseases.

In some embodiments, the lipidomics 204J is a branch of metabolomics that focuses on the comprehensive study and analysis of lipids within a biological system, such as cells, tissues, or organisms. In some embodiments, the lipidomics 204J involves the identification, quantification, and characterization of the diverse array of lipids and their biological roles, interactions, and functions.

In some embodiments, the biological imaging 204K refers to a collection of techniques used to visualize biological processes, structures, and systems in living organisms, tissues, or cells. In some embodiments, these techniques enable researchers and clinicians to study biological phenomena in a non-invasive or minimally invasive way, providing insights into cellular structures, molecular interactions, and physiological functions.

FIG. 2D illustrates steps in obtaining subject variant information 204 through the analysis of genetic information about DNA variants 204A, genetic information about phenotype ranked variants 204B, and differential gene expression transcriptomics 204C, in accordance with some embodiments. In some embodiments, differential gene expression transcriptomics data 204C are collected from the patient samples and are downloaded into data lake 202. In some embodiments, the differential gene expression transcriptomics data characterize how gene expression changes under different biological conditions, for example, healthy vs. diseased tissue, treated vs. untreated cells, different time points, in patient samples. In some embodiments, the techniques for differential gene expression analysis in transcriptomics includes, but is not limited to, RNA sequencing (RNA-seq) 242, Microarrays 244, quantitative PCR (qPCR) 246, Digital droplet PCR (ddPCR) 248, single-cell RNA sequencing (scRNA-seq) 250, Nanostring technology 252, and/or Massively Parallel Reporter Assay (MPRA) 254.

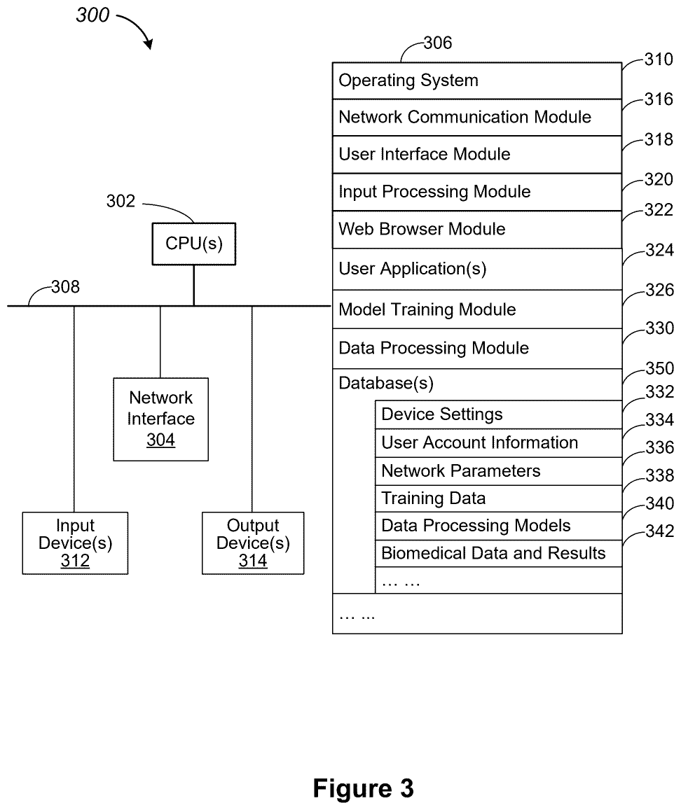

FIG. 3 is a block diagram of a computer system 200 configured to process biomedical data, in accordance with some embodiments. The server 102 includes a server 102A, a client device 140 (e.g., a desktop computer 104A in FIG. 1), a storage 106, or a combination thereof. The server 102A, typically, includes one or more processing units (CPUs) 302, one or more network interfaces 304, memory 306, and one or more communication buses 308 for interconnecting these components (sometimes called a chipset). The server 102 includes one or more input devices 312 that facilitate user input, such as a keyboard, a mouse, a voice-command input unit or microphone, a touch screen display, a touch-sensitive input pad, a gesture capturing camera, or other input buttons or controls. Furthermore, in some embodiments, the client device 140 of the server 102 uses a microphone for voice recognition or a camera for gesture recognition to supplement or replace the keyboard. In some embodiments, the client device 140 includes one or more optical cameras (e.g., an RGB camera), scanners, or photo sensor units for capturing images, for example, of graphic serial codes printed on the electronic devices. The server 102 also includes one or more output devices 314 that enable presentation of user interfaces and display content, including one or more speakers and/or one or more visual displays. Optionally, the client device 140 includes a location detection device, such as a GPS (global positioning system) or other geo-location receiver, for determining the location of the client device 140.

Memory 306 includes high-speed random access memory, such as DRAM, SRAM, DDR RAM, or other random access solid state memory devices; and, optionally, includes non-volatile memory, such as one or more magnetic disk storage devices, one or more optical disk storage devices, one or more flash memory devices, or one or more other non-volatile solid state storage devices. Memory 306, optionally, includes one or more storage devices remotely located from one or more processing units 302. Memory 306, or alternatively the non-volatile memory within memory 306, includes a non-transitory computer readable storage medium. In some embodiments, memory 306, or the non-transitory computer readable storage medium of memory 306, stores the following programs, modules, and data structures, or a subset or superset thereof:
Operating system 310 including procedures for handling various basic system services and for performing hardware dependent tasks;
Network communication module 316 for connecting each server 102 or client device 140 to other devices (e.g., server 102, client device 140, or storage 106) via one or more network interfaces 304 (wired or wireless) and one or more communication networks 108, such as the Internet, other wide area networks, local area networks, metropolitan area networks, and so on;
User interface module 318 for enabling presentation of information (e.g., a graphical user interface for application(s) 324, widgets, websites and web pages thereof, and/or games, audio and/or video content, text, etc.) at each client device 140 via one or more output devices 314 (e.g., displays, speakers, etc.);
Input processing module 320 for detecting one or more user inputs or interactions from one of the one or more input devices 312 and interpreting the detected input or interaction;
Web browser module 322 for navigating, requesting (e.g., via HTTP), and displaying websites and web pages thereof, including a web interface for logging into a user account associated with a client device 140 or another electronic device, controlling the client or electronic device if associated with the user account, and editing and reviewing settings and data that are associated with the user account;
One or more user applications 324 for execution by the server 102 (e.g., games, social network applications, smart home applications, and/or other web or non-web-based applications for controlling another electronic device and reviewing data captured by such devices);
Model training module 326 for obtaining training data and establishing a data processing model 340 for processing biomedical data to be collected or obtained by a server 102A;
Data processing module 330 for processing biomedical data using data processing models 340, thereby identifying information contained in the biomedical data, matching the biomedical data with other data, categorizing the biomedical data, or synthesizing related biomedical data; and
One or more databases 350 for storing at least data including one or more of:
   Device settings 332 including common device settings (e.g., service tier, device model, storage capacity, processing capabilities, communication capabilities, etc.) of the one or more servers 102 or client devices 140;
   User account information 334 for the one or more user applications 324, e.g., usernames, security questions, account history data, user preferences, and predefined account settings;
   Network parameters 336 for the one or more communication networks 108, e.g., IP address, subnet mask, default gateway, DNS server and host name;
   Training data 338 for training one or more data processing models 340;
   Data processing model(s) 340 for processing biomedical data using deep learning techniques; and
   Biomedical data and results 342 that are obtained by the server 102A and outputted to the client device 140, respectively, where the biomedical data is processed by the data processing models 340 to provide the associated results to be presented on client device 140.

Optionally, the one or more databases 350 are stored in one of the server 102, client device 140, and storage 106 of the server 102. Optionally, the one or more databases 350 are distributed in more than one of the server 102, client device 140, and storage 106 of the server 102. In some embodiments, more than one copy of the above data is stored at distinct devices, e.g., two copies of the data processing model 340 are stored at the server 102 and storage 106, respectively.

Each of the above identified elements may be stored in one or more of the previously mentioned memory devices and corresponds to a set of instructions for performing a function described above. The above identified modules or programs (i.e., sets of instructions) need not be implemented as separate software programs, procedures, modules or data structures, and thus various subsets of these modules may be combined or otherwise re-arranged in various embodiments. In some embodiments, memory 306, optionally, stores a subset of the modules and data structures identified above. Furthermore, memory 306, optionally, stores additional modules and data structures not described above.

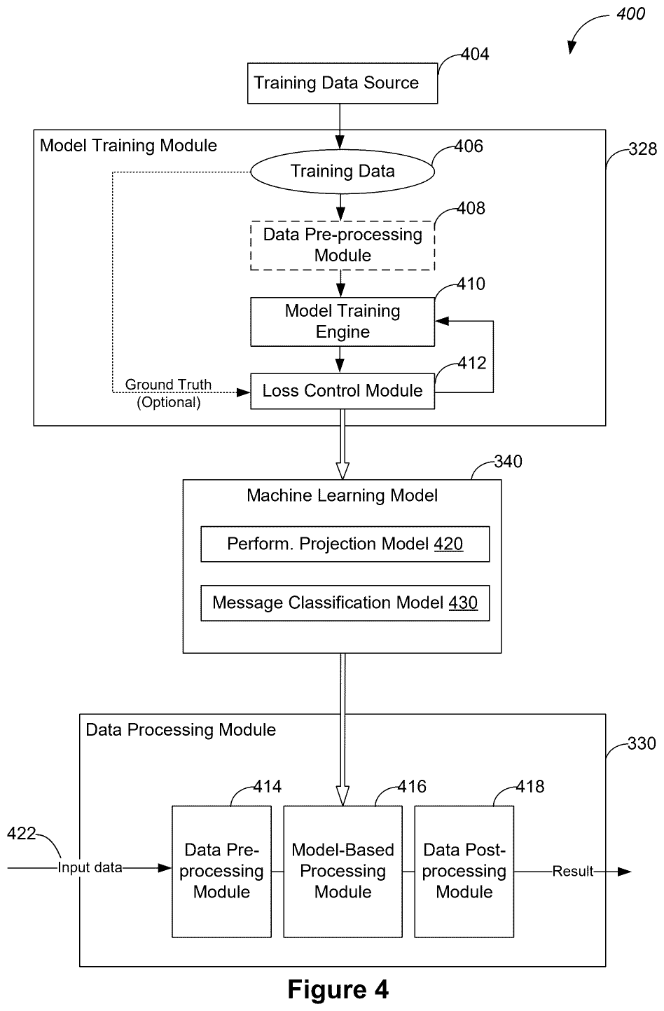

FIG. 4 is an example data processing system 400 for training and applying a neural network based (NN-based) data processing model 340 for processing biomedical data (e.g., video, image, audio, or textual data), in accordance with some embodiments. The data processing system 400 includes a model training module 326 for establishing the data processing model 340 and a data processing module 330 for processing the biomedical data using the data processing model 340. In some embodiments, both of the model training module 326 and the data processing module 330 are located on a client device 140 of the data processing system 400, while a training data source 404 distinct form the client device 140 provides training data 338 to the client device 140. The training data source 404 is optionally a server 102 or storage 106. Alternatively, in some embodiments, both of the model training module 326 and the data processing module 330 are located on a server 102 of the data processing system 400. The training data source 404 providing the training data 338 is optionally the server 102 itself, another server 102, or the storage 106. Additionally, in some embodiments, the model training module 326 and the data processing module 330 are separately located on a server 102 and client device 140, and the server 102 provides the trained data processing model 340 to the client device 140.

The model training module 326 includes one or more data pre-processing modules 408, a model training engine 410, and a loss control module 412. The data processing model 340 is trained according to a type of the biomedical data to be processed. The training data 338 is consistent with the type of the biomedical data, so is a data pre-processing module 408 applied to process the training data 338 consistent with the type of the biomedical data. For example, an image pre-processing module 408A is configured to process image training data 338 to a predefined image format, e.g., extract a region of interest (ROI) in each training image, and crop each training image to a predefined image size. Alternatively, an audio pre-processing module 408B is configured to process audio training data 338 to a predefined audio format, e.g., converting each training sequence to a frequency domain using a Fourier transform. The model training engine 410 receives pre-processed training data provided by the data pre-processing modules 408, further processes the pre-processed training data using an existing data processing model 340 and generates an output from each training data item. During this course, the loss control module 412 can monitor a loss function comparing the output associated with the respective training data item and a ground truth of the respective training data item. The model training engine 410 modifies the data processing model 340 to reduce the loss function, until the loss function satisfies a loss criteria (e.g., a comparison result of the loss function is minimized or reduced below a loss threshold). The modified data processing model 340 is provided to the data processing module 330 to process the biomedical data.

In some embodiments, the model training module 326 offers supervised learning in which the training data is entirely labelled and includes a desired output for each training data item (also called the ground truth in some situations). Conversely, in some embodiments, the model training module 326 offers unsupervised learning in which the training data are not labelled. The model training module 326 is configured to identify previously undetected patterns in the training data without pre-existing labels and with no or little human supervision. Additionally, in some embodiments, the model training module 326 offers partially supervised learning in which the training data are partially labelled.

The data processing module 330 includes a data pre-processing modules 414, a model-based processing module 416, and a data post-processing module 418. The data pre-processing modules 414 pre-processes the biomedical data based on the type of the biomedical data. Functions of the data pre-processing modules 414 are consistent with those of the pre-processing modules 408 and covert the biomedical data to a predefined content format that is acceptable by inputs of the model-based processing module 416. Examples of the biomedical data include one or more of: video, image, audio, textual, and other types of data. For example, each image is pre-processed to extract an ROI or cropped to a predefined image size, and an audio clip is pre-processed to convert to a frequency domain using a Fourier transform. In some situations, the biomedical data includes two or more types, e.g., video data and textual data. The model-based processing module 416 applies the trained data processing model 340 provided by the model training module 326 to process the pre-processed biomedical data. The model-based processing module 416 can also monitor an error indicator to determine whether the biomedical data has been properly processed in the data processing model 340. In some embodiments, the processed biomedical data is further processed by the data post-processing module 418 to present the processed biomedical data in a preferred format or to provide other related information that can be derived from the processed biomedical data.

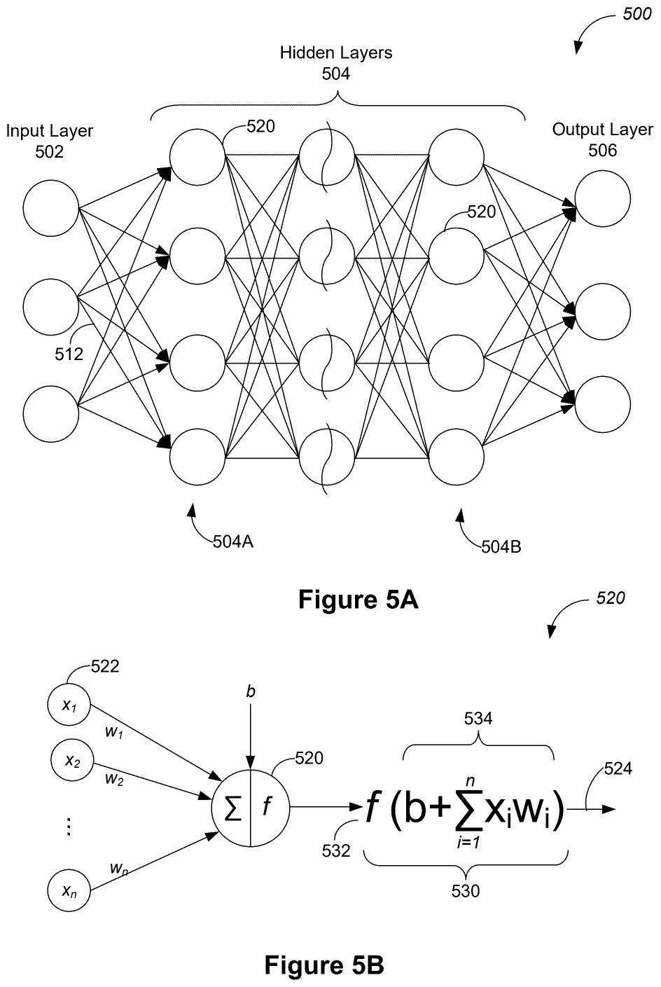

FIG. 5A is an example neural network (NN) 500 applied to process biomedical data in an NN-based data processing model 340, in accordance with some embodiments, and FIG. 5B is an example node 520 in the neural network (NN) 500, in accordance with some embodiments. The data processing model 340 is established based on the neural network 500. A corresponding model-based processing module 416 applies the data processing model 340 including the neural network 500 to process biomedical data that has been converted to a predefined content format. The neural network 500 includes a collection of nodes 520 that are connected by links 512. Each node 520 receives one or more node inputs and applies a propagation function to generate a node output from the one or more node inputs. As the node output is provided via one or more links 512 to one or more other nodes 520, a weight w associated with each link 512 is applied to the node output. Likewise, the one or more node inputs are combined based on corresponding weights w₁, w₂, w₃, and w₄ according to the propagation function. In an example, the propagation function is a product of a non-linear activation function and a linear weighted combination of the one or more node inputs.

The collection of nodes 520 is organized into one or more layers in the neural network 500. Optionally, the one or more layers includes a single layer acting as both an input layer and an output layer. Optionally, the one or more layers includes an input layer 502 for receiving inputs, an output layer 506 for providing outputs, and zero or more hidden layers 504 (e.g., 504A and 504B) between the input and output layers 502 and 506. A deep neural network has more than one hidden layers 504 between the input and output layers 502 and 506. In the neural network 500, each layer is only connected with its immediately preceding and/or immediately following layer. In some embodiments, a layer 502 or 504B is a fully connected layer because each node 520 in the layer 502 or 504B is connected to every node 520 in its immediately following layer. In some embodiments, one of the one or more hidden layers 504 includes two or more nodes that are connected to the same node in its immediately following layer for down sampling or pooling the nodes 520 between these two layers. Particularly, max pooling uses a maximum value of the two or more nodes in the layer 504B for generating the node of the immediately following layer 506 connected to the two or more nodes.

In some embodiments, a convolutional neural network (CNN) is applied in a data processing model 340 to process biomedical data (particularly, video and image data). The CNN employs convolution operations and belongs to a class of deep neural networks 500, i.e., a feedforward neural network that only moves data forward from the input layer 502 through the hidden layers to the output layer 506. The one or more hidden layers of the CNN are convolutional layers convolving with a multiplication or dot product. Each node in a convolutional layer receives inputs from a receptive area associated with a previous layer (e.g., five nodes), and the receptive area is smaller than the entire previous layer and may vary based on a location of the convolution layer in the convolutional neural network. Video or image data is pre-processed to a predefined video/image format corresponding to the inputs of the CNN. The pre-processed video or image data is abstracted by each layer of the CNN to a respective feature map. By these means, video and image data can be processed by the CNN for video and image recognition, classification, analysis, imprinting, or synthesis.

Alternatively, and additionally, in some embodiments, a recurrent neural network (RNN) is applied in the data processing model 340 to process biomedical data (particularly, textual and audio data). Nodes in successive layers of the RNN follow a temporal sequence, such that the RNN exhibits a temporal dynamic behavior. In an example, each node 520 of the RNN has a time-varying real-valued activation. Examples of the RNN include, but are not limited to, a long short-term memory (LSTM) network, a fully recurrent network, an Elman network, a Jordan network, a Hopfield network, a bidirectional associative memory (BAM network), an echo state network, an independently RNN (IndRNN), a recursive neural network, and a neural history compressor. In some embodiments, the RNN can be used for handwriting or speech recognition. It is noted that in some embodiments, two or more types of biomedical data are processed by the data processing module 330, and two or more types of neural networks (e.g., both CNN and RNN) are applied to process the biomedical data jointly.

The training process is a process for calibrating of the weights wᵢ for each layer of the learning model using a training data set which is provided in the input layer 502. The training process typically includes two steps, forward propagation and backward propagation, which are repeated multiple times until a predefined convergence condition is satisfied. In the forward propagation, the set of weights for different layers are applied to the input data and intermediate results from the previous layers. In the backward propagation, a margin of error of the output (e.g., a loss function) is measured, and the weights are adjusted accordingly to decrease the error. The activation function is optionally linear, rectified linear unit, sigmoid, hyperbolic tangent, or of other types. In some embodiments, a network bias term b is added to the sum of the weighted outputs from the previous layer before the activation function is applied. The network bias b provides a perturbation that helps the NN 500 avoid over fitting the training data. The result of the training includes the network bias parameter b for each layer.

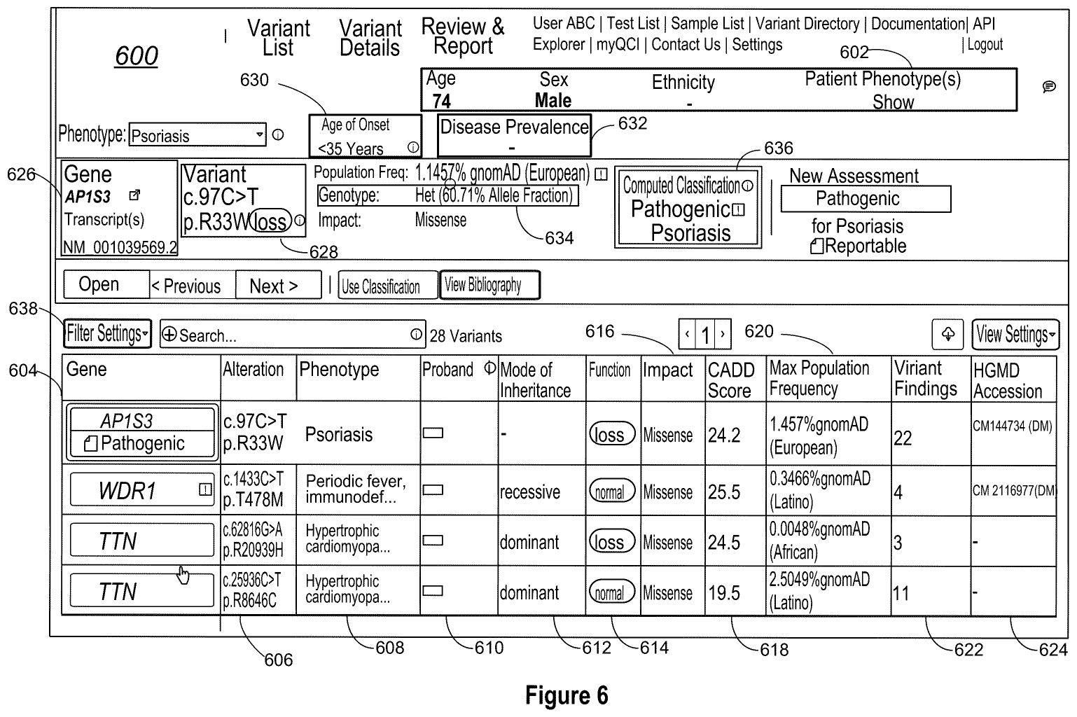

FIG. 6 illustrates ranked variants based the correlations of variants with the phenotype of the targeted diseases obtained from tertiary analysis displayed on a user interface 600, in accordance with some embodiments. In some embodiments, the variants are ranked based the correlations of variants with the phenotype of the targeted diseases obtained from tertiary analysis. In some embodiments, a filter 638 is applied to select the subset of subject genetic variants for ranking from the plurality of subject genetic variants based on one or more of: a confidence score, a population frequency of occurrence, common variant, a predicted deleterious level, or a biological impact. In some embodiments, a higher ranking is associated with a greater likelihood of being pathogenic for a selected phenotype of the targeted diseases. In some embodiments, information about the ranked variants is displayed on a user interface. In some embodiments, the displayed information about the ranked variants comprises patient information 602, phenotype ranked variants 604, gene alteration loci 606, phenotype 608, proband status 610, mode of inheritance (MOI) 612, loss-of-function status 614, mutation impact 616, CADD (Combined Annotation Dependent Depletion) score 618, Max population frequency 620, variant findings 622, and human gene mutation database (HGMD), or other databases such as ClinVar, ClinGen, etc, accession 624. In some embodiments, for each ranked variant for a selected phenotype of the targeted diseases, gene information including a gene symbol and transcripts information 626, mutation loci 628, age of onset 630, disease prevalence 632, zygosity 634, and predicted pathogenicity of variant 636 are available on the interface.

In some embodiments, the ranked variants information 604-640 are obtained through a clinical decision support software that accelerates variant interpretation and reporting of germline, hereditary and oncology NGS tests at scale. In some embodiments, the software is powered by insights about symptoms, phenotypes and gene-disease associations, biomedical databases such as HGMD, ClinVar, Clingen, VarElect, COSMIC, etc medical guidelines and a wide variety of different bibliography content sources that are clinically relevant and are manually curated. In some embodiments, the clinical decision support software computes and combines the relevant information related to the variant of interest and distributes the relevant biological context. In some embodiments, the clinical decision support software uses more than 20 million curated findings and evidence and has analyzed more than 3 million clinical cases supported by AI and Augmented Molecular Insights. In some embodiments, the clinical decision support software offers the possibility of phenotype driven analysis where the user can submit phenotypes or symptoms of suspected disease or disease under investigation along with the VCF file 236 of the sample. Based on this info, the phenotype-driven ranking algorithm in the clinical decision support software estimates and ranks genomic variants based on the probability of being the causative one for the disease, symptoms, or the phenotypes under investigation by taking into account multiple variables such as zygosity 636, predicted pathogenicity of variant 636, MOI (mode of inheritance) 612, CADD score 618, and more variant-centric variables as well as other curated molecular insights.

FIGS. 7A and 7B illustrate a flowchart illustrating a method 700 for identifying disease-specific genetic variants, in accordance with some embodiments. In some embodiments, information of a plurality of subject genetic variants that are identified from a plurality of biological samples of a plurality of patients who are diagnosed with a target disease are obtained (operation 702). In some embodiments, patient samples 222 (FIG. 2B) are taken in a clinical diagnostic setting. In various embodiments, patient samples 222 include blood, saliva, or other cell types for DNA 224 or a combination thereof. Genetic information is extracted from the patient samples 226. In some embodiments, a library 228 is prepared from the patient samples 222 by any available sequencing platforms for high throughput, large-scale parallel sequencing. In some embodiments, whole exome or whole genome NGS 230 is performed on each sample to determine the presence or absence of known pathogenic mutations, and variants of unknown clinical significance (VUS) associated with targeted diseases. In some embodiments, clinical exome 232 ordered by physicians with a signed informed consent are being analyzed. In some embodiments, secondary analysis 234 is performed on the sequencing data obtained from, e.g., whole genome sequencing or clinical exomes sequencing, where the secondary analysis compares a reference genome to the patient samples for generating genome information of a plurality of subject genetic variants identified from the plurality of patients associated with the targeted disease. More details on patient samples 222 are explained above with reference FIG. 2B. In some embodiments, information of a plurality of subject genetic variants that are identified from a plurality of patient samples 222 includes datasets obtained from one or more additional omics analysis. In some embodiments, the one or more additional omics analysis include, but are not limited to, differential gene expression transcriptomics 204C, epigenetics 204D, proteomics 204E, metabolomics 204F, pharmacogenomics 204G, optical genome mapping (OGM) 204H, spatial transcriptomics 204I, lipidomics 204J, and/or biological imaging 204K (FIG. 2C). In some embodiments, subject variant information 204 is obtained through the analysis of the combination of genetic information about DNA variants 204A, genetic information about phenotype ranked variants 204B, and differential gene expression transcriptomics 204C (FIG. 2D). In some embodiments, subject variant information 204A-204K or any combination thereof, are all downloaded into a data lake 202.

In some embodiments, the disease includes, but is not limited to, endometriosis, endometrial cancer, or an endometrial form of ovarian cancer, rheumatoid arthritis, osteoarthritis or other autoimmune disorders. In some embodiments, the disease is a type of genetic disease, including, but not limited, Alzheimer's disease (APOE1). Charcot-Marie-Tooth disease, Leber hereditary optic neuropathy (LHON), Angelman syndrome (UBE3A, ubiquitin-protein ligase E3A), Prader-Willi syndrome (region in chromosome 15), β-Thalassaemia (HBB, β-Globin), Gaucher disease (type I) (GBA, Glucocerebrosidase), Cystic fibrosis (CFTR Epithelial chloride channel), Sickle cell disease (HBB, β-Globin), Tay-Sachs disease (HEXA, Hexosaminidase A), Phenylketonuria (PAH, Phenylalanine hydrolyase), Familial hypercholesterolemia a (LDLR, Low density lipoprotein receptor), Adult polycystic kidney disease (PKD1, Polycystin), Huntington disease (HDD, Huntingtin), Neurofibromatosis type (NF1, NF1 tumor suppressor gene), Myotonic dystrophy (DM, Myotonin), Tuberous sclerosis (TSC1, Tuberin), Achondroplasia (FGFR3, Fibroblast growth factor receptor), Fragile X syndrome (FMRT, RNA-binding protein), Duchenne muscular dystrophy (DMD, Dystrophin), Haemophilia A (F8C, Blood coagulation factor VIII), Lesch-Nyhan syndrome (HPRT1, Hypoxanthine guanine ribosyltransferase 1), autistic like spectrum, rare diseases, multifactorial disorders, and Adrenoleukodystrophy (ABCD1).

In some embodiments, a subset of subject genetic variants is selected from the plurality of subject genetic variants based on a plurality of subject phenotypes of the target disease (operation 704). In some embodiments, to obtain a subset of subject genetic variants, tertiary analysis 238 (FIG. 2B) is performed on the results obtained from secondary analysis, optionally in combination with the any outside VCF files 236 about the genome information of variants from the patients. In some embodiments, tertiary analysis 238 interprets the variant information based on knowledge about symptoms, phenotypes and gene-disease associations, from biomedical databases, medical guidelines and a wide variety of different bibliography content sources that are clinically relevant. In some embodiments, a filter 638 is applied to select the subset of subject genetic variants from the plurality of subject genetic variants based on one or more of: a confidence score, a population frequency of occurrence, a predicted deleterious level, or a biological context. More details on tertiary analysis 238 are explained above with reference FIG. 6.

In some embodiments, the subset of subject genetic variants are ranked based on the plurality of subject phenotypes to generate subject genetic variant information (operation 706). In some embodiments, the variants are ranked based the correlations of variants with the phenotype of the targeted diseases obtained from tertiary analysis 238 (FIG. 2B). In some embodiments, genetic information about DNA variants 204A and phenotype ranked variants 204B are downloaded into a data lake 202 (FIG. 2B).

In some embodiments, the method 700 includes obtaining subject medical information of the plurality of patients, including doctor-inputted description of the target disease collected from the plurality of patients (operation 708). In some embodiments, the subject medical information includes, but is not limited to, patient medical record, EMR data 206, family history of targeted diseases, and medical information of the plurality of patients (FIG. 2B). The subject medical information is exported to the data lake 202.

In some embodiments, the method 700 includes (operation 710) obtaining general genetic variant information, independently of the plurality of patients. In some embodiments, the general genetic variant information includes a first knowledge graph that couples the target disease and a plurality of first biomedical terms semantically to one another based on a public knowledge database. In some embodiments, the public knowledge database includes a collection of official peer-reviewed publications. In some embodiments, the first knowledge graph is generated by semantically analyzing a subset of the collection of official peer-reviewed publications, extracting the plurality of first biomedical terms that are mentioned in the subset of official publications jointly with the target disease, and in accordance with an analysis of the official peer-reviewed publications, forming the first knowledge graph including connecting the target disease directly or indirectly with each of the plurality of first biomedical terms. In some embodiments, the general genetic variant information includes a second knowledge graph that couples the target disease and a plurality of second biomedical terms semantically to one another based on a private knowledge database. In some embodiments, the private knowledge database includes information items collected from a group of subject experts including a set of experts of the target disease. In some embodiments, the second knowledge graph is generated by semantically analyzing a subset of information items, extracting the plurality of second biomedical terms that are mentioned in the subset of information items jointly with the target disease, and in accordance with a semantic analysis of the information items, forming the second knowledge graph including connecting the target disease directly or indirectly with each of the plurality of second biomedical terms. In some embodiments, the first knowledge graph is a knowledge graph 208 derived from research databases, such as PubMed or HGMD, and the second knowledge graph is a “Expert Knowledge graph” designed by experts in the disease studied 210 (FIG. 2A).

In some embodiments, the operation 710 further includes obtaining general genetic variant information about annotated and predicted human genes through extracting, from a first database, the general gene information of the genomic related information that is associated with the plurality of subject genetic variants. In some embodiments, the first database is a public first database. In some embodiments, the first public database is a searchable, integrative database that provides information on annotated and predicted human genes 212 A. The database 212A will automatically integrate gene-centric data from ˜300 web sources, including genomic, transcriptomic, proteomic, genetic, clinical, and functional information (FIG. 2A).

In some embodiments, the operation 710 further includes obtaining general genetic variant information about associations between human gene variants and phenotypes through obtaining, from a second database, the associations between human gene variants and phenotypes, where the corresponding general genetic variants are prioritized based on their corresponding associations with a plurality of general phenotypes of the target disease. In some embodiments, the obtaining the associations between human gene variants and phenotypes further includes providing a query to the second public database, the query identifying the target disease, and in response to the query, extracting the information about the associations between human gene variants and phenotypes from the second public database. In some embodiments, the second database 212B is phenotype-dependent DNA variant/gene prioritizer, to identify causal DNA variants with phenotypes (genotype/phenotype). The second database provides search and scoring capabilities, proficiently matching DNA variant-containing genes to submitted disease, symptom, or phenotype keywords. This output will infer direct as well as indirect links between genes and phenotypes (FIG. 2A).

In some embodiments, the operation 710 obtains one or more of: a first knowledge graph that couples the target disease and a plurality of first biomedical terms semantically to one another based on a public knowledge database, a second knowledge graph that couples the target disease and a plurality of second biomedical terms semantically to one another based on a private knowledge database, general gene information of a plurality of human genes which includes at least a subset of first genes that are associated with the plurality of subject genetic variants, and genetic variant information that identifies and prioritizes corresponding general genetic variants based on a plurality of general phenotypes of the target disease. In some embodiments, general genetic variant information is exported to the data lake 202 (FIG. 2A).

In some embodiments, the method 700 includes applying an information model to process the subject genetic variant information, the subject medical information, and the general genetic variant information of the target disease (712). In some embodiments, the biomedical information model includes a neural network or a set of predefined information processing rules.

In some embodiments, the applying the biomedical information model (operation 712) includes quantitatively determining a set of transcription factors, a set of translation factors, a plurality of subject factors, a plurality of genetic factors, and a plurality of protein factors associated with the subset of subject genetic variants (operation 714), and generating a score for the subset of subject genetic variants using a weighted combination of the set of transcription factors, the set of translation factors, the plurality of subject factors, the plurality of genetic factors, and the plurality of protein factors. The variant selection criterion requires that the set of target genetic variants be ranked among a predefined number of subject genetic variants having the highest score (operation 716).

In some embodiments, the applying the biomedical information model (operation 712) includes quantitatively determining the percentages of patients carrying the subject genetic variants, the correlations of the subject genetic variants with patient's medical history, the correlations of the subject genetic variants to the generated knowledge graphs, and the correlations of the subject genetic variants to phenotypes of the targeted disease based on first and second public databases, and generating a score for the subset of subject genetic variants using a weighted combination of the percentages of patients carrying the subject genetic variants, the correlations of the subject genetic variants with patient's medical history, the correlations of the subject genetic variants to the generated knowledge graphs, and the correlations of the subject genetic variants to phenotypes of the targeted disease based on first and second public databases, where the variant selection criterion requires that the set of target genetic variants be ranked among a predefined number of subject genetic variants having the highest score.

In some embodiments, the applying a biomedical information model (operation 712) is applying a Biomimetic Digital Twins engine 214, where the data from the data lake 202 is exported into the Biomimetic Digital Twins engine for analysis. Each twin model is a discrete component of the analytical scope of the ecosystem and each twin can initiate an interaction with others or respond as prompted in an automated manner.

In some embodiments, the method 700 includes identifying a set of target genetic variants from the subset of subject genetic variants, where the set of target genetic variants satisfies a variant selection criterion (operation 718). In some embodiments, the set of target genetic variants includes a first number of genetic variants associated with the target disease, and the subset of subject genetic variants includes a second number of genetic variants associated with the target disease, the first number is at least two orders smaller than the second number. In some embodiments, each of the subset of subject genetic variants is detected in samples of a respective number of patients in the plurality of patients. The subject genetic variant information includes the respective number of patients corresponding to each variant of the subset of subject genetic variants. The subset of subject genetic variants is ranked partially based on the respective number of patients corresponding to each of the subset of subject genetic variants. In some embodiments, the set of target genetic variants includes a first subset of target genetic variants that are directly linked to the target disease and a second subset of target genetic variants that are indirectly linked to the target disease.

In some embodiments, Digital Twin Ecosystem's biomimetic engine produced a list of gene variants classified as VUSs 218 potentially associated with the targeted disease (FIG. 2B), where VUSs are assigned a genotype-phenotype ranking. In some embodiments, VUSs are ranked according to the number of times that they are present in patient samples 222 but absent from controls. In some embodiments, the target disease includes endometriosis and endometriosis-related infertility, and the list of gene variants classified as VUSs includes MUC 20, USP17L1, FAM66B, and DEFB109B. In some embodiments, the target disease includes Rheumatoid Arthritis, and the list of gene variants classified as VUSs includes HIF1A, HLA-DOA, PTGER3, HIPK3, TGFBR3 and HIF1A-AS3.

In some embodiments, the list of VUS 218 includes the genome information of the plurality of subject genetic variants includes information of an outlier genetic variant, a reclassified outlier VUS 220, e.g., corresponding to the set of target genetic variants. The information of the reclassified outlier VUS 220 is not included in the general genetic variant information, and the reclassified outlier VUS 220 is preserved in the set of target genetic variants after the biomedical information model is applied and applied to identify the set of target genetic variants.

In some embodiments, the outliner variants include reclassified outlier VUS 220 hidden from identification from traditional AI/ML/LLM approaches. In some embodiments, the target disease includes endometriosis and endometriosis-related infertility, and the reclassified outlier VUS 220 includes MUC 20, USP17L1, FAM66B, and DEFB109B. In some embodiments, the target disease includes Rheumatoid Arthritis, and the reclassified outlier VUS 220 includes HIF1A, HLA-DOA, PTGER3, HIPK3, TGFBR3 and HIF1A-AS3.

In some embodiments, the method 700 further includes determining that the general genetic variant information is updated, and in accordance with a determination that the general genetic variant information is updated, re-applying the biomimetic information model to update the set of target genetic variants (operation 720).

In some embodiments, the method 700 further includes ranking the set of target genetic variants based on a correlation level with the plurality of subject phenotypes of the target disease, and in accordance with ranking, associating each of the set of target genetic variants with one of a plurality of predefined genetic significance levels (operation 722). In some embodiments, the plurality of predefined genetic significance levels includes pathogenic, likely pathogenic, benign, and likely benign. In some embodiments, the correlation level with the plurality of subject phenotypes of the target disease is obtained by multiple steps and considerations, including, but not limited to gather clinical information about the individual who underwent genetic testing, classifying variants according to established guidelines, performing functional studies, obtaining population frequency of variants, and/or in silico predicting the functional impact of a variant. In some embodiments, outlier VUS can be reclassified 220 based on the obtained correlation level with the plurality of subject phenotypes of the target disease (FIG. 2A).

In some embodiments, a proteomics workflow 258 may be added to the process to predict functional vs non-functional proteins. In some embodiments, the proteomics workflow may include performing sequence to structure and function predictions, analyzing the VUS in context of proteins, and predicting if there is any impact on the structure of the protein because of the variant.

In some embodiments, the method 700 further includes assessing the set of target variants as therapeutic targets and determining one or more compounds of a drug configured to treat the target disease (operation 724). In some embodiments, the set of target variants includes a first target variant, and assessing the set of target variants further including one or more of: gathering clinical information about the plurality of patients, reclassifying the first target variant to one of a set of classes including pathogenic, likely pathogenic, uncertain significance, likely benign, and benign, conducting functional study to assess an impact of the first target variant on a protein function or expression, assessing a frequency of the first target variant in one or more populations, determining whether the first target variant co-segregates with a predefined phenotype, predicting a functional impact of the first target variant based on a variant location within a corresponding gene and an effect on a protein structure, and/or identifying a pathway affected by the first target variant and contributing to one or more phenotypes of the target disease. In some embodiments, the one or more compounds of the drug include small molecules, antibodies, gene therapies, an immunotherapy, a chemotherapy, a radiation therapy, a hormone therapy, a photodynamic therapy, a targeted therapy, polynucleotide, natural compound, immune modulator, bone marrow therapy, stem cell therapy, surgery therapy, induction therapy, maintenance therapy, or a combination thereof. In some embodiments, reclassifying outlier VUSs to one of a set of classes (pathogenic, likely pathogenic, uncertain significance, likely benign, and benign) reduces the time and cost of drug development 256 (FIG. 2A). In some embodiments, a drug discovery pipeline can be added to the drug development process 256, including, but are not limited to, Integrated Modeling PipelinE for COVID Cure by Assessing Better LEads (IMPECCABLE). The IMPECCABLE pipeline for drug discovery, basically utilizes virtual screening and MD simulation-based energy calculations to determine the high scoring compounds against the target protein. In some embodiments, the drug discovery pipeline utilizes supercomputer facilities to perform virtual screening against the target protein to develop a potential therapeutic target. In some embodiments, LLMs can also be included into this framework so the therapeutics can be classified based on their available information and the drug discovery pipeline can be developed further regarding other therapeutics currently available for the disease studied.

In one or more examples, the functions described may be implemented in hardware, software, firmware, or any combination thereof. When implemented in software, the functions may be stored on or transmitted over, as one or more instructions or code, on a computer-readable storage medium, and executed by a hardware-based processing unit. Computer-readable storage media may include computer-readable storage media, which corresponds to a tangible medium such as a data storage medium, or communication media including any medium that facilitates transfer of a computer program from one place to another (e.g., according to a communication protocol). In this manner, computer-readable storage media generally may correspond to (1) tangible computer-readable storage media, which is non-transitory or 2) a communication medium such as a signal or carrier wave. Data storage media may be any available media that can be accessed by one or more computers or one or more processors to retrieve instructions, code and/or data structures for implementation of the embodiments described in the present application. A computer program product may include a computer-readable medium.

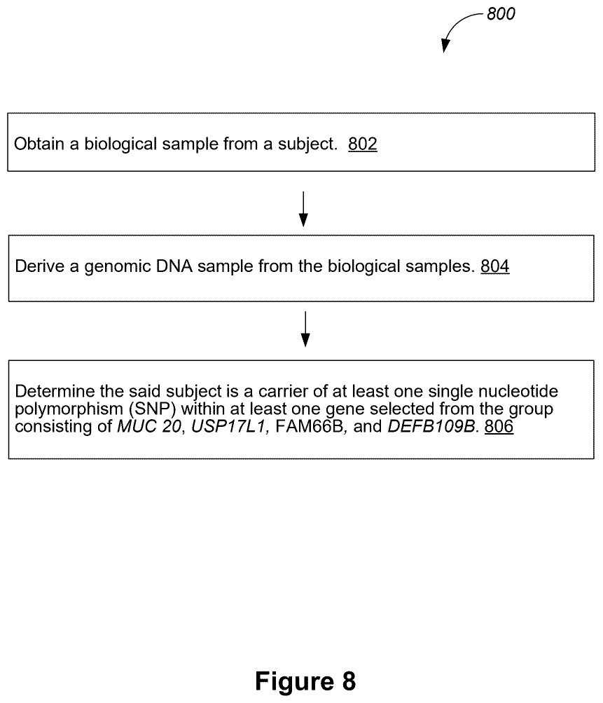

FIG. 8 is a flowchart illustrating a method of identifying a subject having a high risk of endometriosis, endometrial cancer, or an endometrial form of ovarian cancer. The method including firstly obtaining a biological sample from a subject (operation 802). In some embodiments, the subjects are genetically susceptible individuals to these diseases. A genomic DNA sample is derived from the biological sample (operation 804). In various embodiments, the sample is saliva, whole blood, blood serum, blood plasma, urine, saliva, other body fluid or biofluid, cell sample, or tissue sample, or a combination thereof. In various embodiments, the sample includes a nucleic acid from the subject. The sample is being processed to determine whether the said subject is a carrier of at least one variant within at least one gene selected from the group consisting of MUC20, USP17L1, FAM66B, and DEFB109B (operation 806). In some embodiments, determining the said subject is a carrier of at least one variant of the targeted genes includes detecting a gene sequence of a variant at the targeted gene/genetic locus. In some embodiments, the targeted gene/genetic locus includes MUC 20, USP17L1, FAM66B, or DEFB109B, or a combination thereof. In some embodiments, the variant SNP within the gene MUC20 is at the location of rs10794288 or rs10902088. In some embodiments, the determining the said subject is a carrier of at least one variant is performed by a genotyping assay, polymerase chain reaction (PCR), reverse transcription PCR, quantitative PCR, a microarray, DNA sequencing, and/or RNA sequencing. In yet other embodiments, genotyping assay includes contacting the sample with an oligonucleotide probe specific to a variant allele of MUC 20, USP17L1, FAM66B, or DEFB109B, generating an allele-specific hybridization complex between the oligonucleotide probe and the variant allele, and upon detecting the allele-specific hybridization complex, detecting the variant allele, or upon not detecting the allele-specific hybridization complex, not detecting the variant allele. Upon detecting at least one variant allele of MUC 20, USP17L1, FAM66B, and DEFB109B in a subject, the method further includes administering one or more therapeutic agents compounds to said subject for the treatment of endometriosis, endometrial cancer, or an endometrial form of ovarian cancer.

In some embodiments, the present invention also provides a process for identifying a subject having a high risk of endometriosis, endometrial cancer, or an endometrial form of ovarian cancer. The process may include one or more detection agents for detecting one or more variant alleles at one or more genes/genetic location of MUC20, USP17L1, FAM66B, and DEFB109B. In some embodiments, the process includes at least one probe to selectively hybridizes to at least one nucleotide variant under high stringency conditions and amplifies the nucleotide variant sequence but does not amplify a corresponding wild-type sequence, where the nucleotide variant includes at least one DNA variant within at least one gene selected from the group consisting of MUC 20, USP17L1, FAM66B, and DEFB109B.

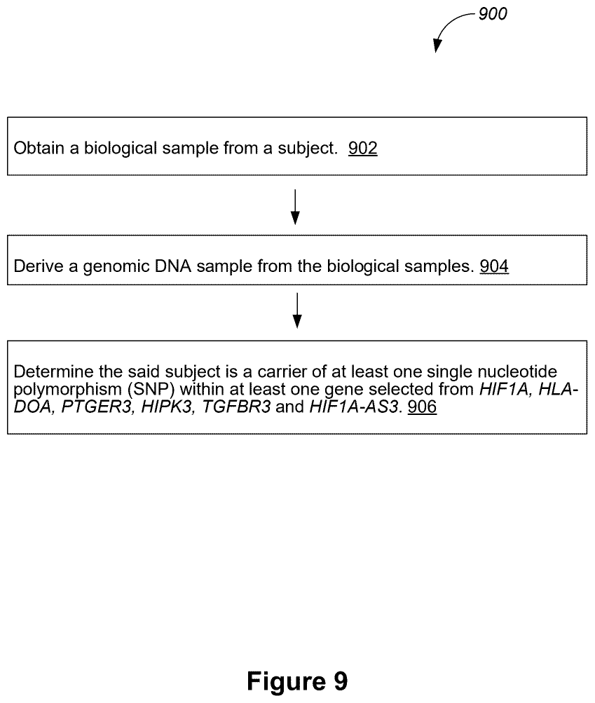

FIG. 9 is a flowchart illustrating a method of identifying a subject having a high risk of rheumatoid arthritis. The method including first obtaining a biological sample from a subject (operation 902). In some embodiments, the subjects are diagnosed or genetically susceptible to rheumatoid arthritis. A genomic DNA sample is derived from the biological sample (operation 904). In various embodiments, the sample is saliva, whole blood, blood serum, blood plasma, urine, saliva, other body fluid or biofluid, cell sample, or tissue sample, or a combination thereof. In various embodiments, the sample includes a nucleic acid from the subject. The sample is being processed to determine whether the said subject is a carrier of at least one single DNA variant within at least one gene selected from the group consisting of HIF1A, HLA-DOA, PTGER3, HIPK3, TGFBR3 and HIF1A-AS3 (operation 906). In some embodiments, determining the said subject is a carrier of at least one variant of the targeted genes includes detecting a gene sequence of a variant at the targeted gene/genetic locus. In some embodiments, the targeted gene/genetic locus includes HIF1A, HLA-DOA, PTGER3, HIPK3, TGFBR3 or HIF1A-AS3, or a combination thereof. In some embodiments, the DNA variant/genetic locus are shown as in Table 4. In some embodiments, the determining the said subject is a carrier of at least one DNA variant is performed by a genotyping assay, polymerase chain reaction (PCR), reverse transcription PCR, quantitative PCR, a microarray, DNA sequencing, and/or RNA sequencing. In yet other embodiments, genotyping assay includes contacting the sample with an oligonucleotide probe specific to a SNP allele of HIF1A, HLA-DOA, PTGER3, HIPK3, TGFBR3 or HIF1A-AS3; generating an allele-specific hybridization complex between the oligonucleotide probe and the allele; and upon detecting the allele-specific hybridization complex, detecting the allele; or upon not detecting the allele-specific hybridization complex, not detecting the allele. Upon detecting at least one allele of HIF1A, HLA-DOA, PTGER3, HIPK3, TGFBR3 or HIF1A-AS3, the method further includes administering one or more therapeutic agents compounds to said subject for the treatment of rheumatoid arthritis.

In some embodiments, the present invention also provides a process for identifying a subject having a high risk of rheumatoid arthritis. The process may include one or more detection agents for detecting one or more alleles at one or more genes/genetic location of HIF1A, HLA-DOA, PTGER3, HIPK3, TGFBR3 or HIF1A-AS3. In some embodiments, the process includes at least one probe to selectively hybridizes to at least one nucleotide variant under high stringency conditions and amplifies the nucleotide variant sequence but does not amplify a corresponding wild-type sequence, where the nucleotide variant includes at least one allele within at least one gene selected from the group consisting of HIF1A, HLA-DOA, PTGER3, HIPK3, TGFBR3 and HIF1A-AS3. General genetic variant information includes genomic related information on annotated and predicted human genes.

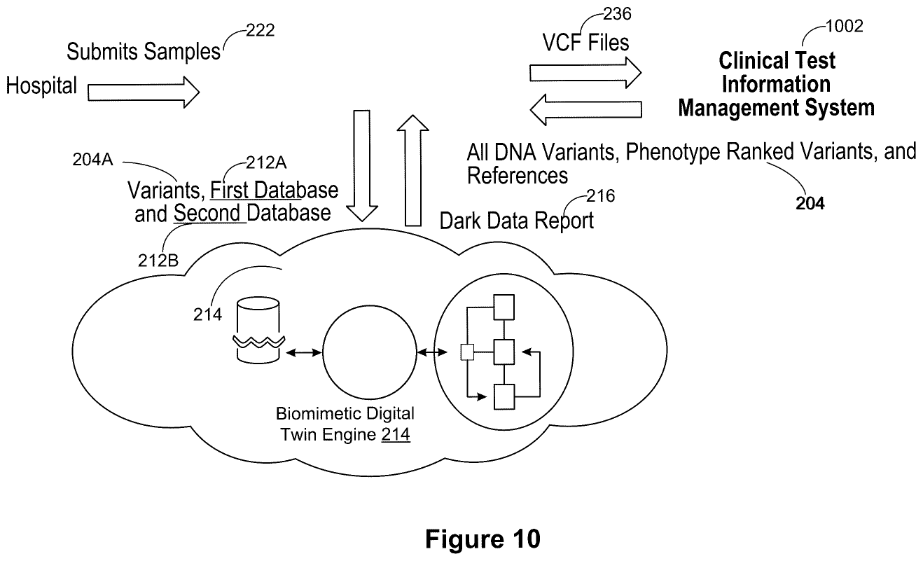

FIG. 10 illustrates an example biomimetic digital twin ecosystem process 1000, in accordance with some embodiments. In some embodiments, patient samples 222 are submitted for sequencing to obtain genome information of a plurality of subject genetic variants that are identified from a plurality of biological samples of a plurality of patients who are diagnosed with a target disease. In some embodiments, each patient's DNA and matched control underwent exome sequencing or whole genome sequencing, and secondary analysis 234. In some embodiments, VCF files 236 contains information about variants found at specific positions in a reference genome are obtained after a secondary analysis 234. In some embodiments, a subset of subject genetic variants from the plurality of subject genetic variants are selected based on a plurality of subject phenotypes of the target diseases. In some embodiments, the subset of subject genetic variants are ranked by a clinical test information management system 1002 based on the plurality of subject phenotypes to generate subject variant information 204. In some embodiments, general genetic variant information, independently of the plurality of patients are obtained. In some embodiments, general genetic variant information independently of the plurality of patients includes genomic related information on annotated and predicted human genes 212A. In some embodiments, general genetic variant information independently of the plurality of patients includes information about associations between human gene variants and phenotypes (e.g., in a second database 212B). In some embodiments, the digital twin ecosystem's biomimetic engine 214 combined and processed data, including subject variant information 204, annotated and predicted human genes (e.g., stored in a first database 212A), and information about associations between human gene variants and phenotypes (e.g., in a second database 212B). In some embodiments, the digital twin ecosystem's biomimetic engine 214 identified a set of target genetic variants from the subset of subject genetic variants, wherein the set of target genetic variants satisfying a variant selection criterion. In some embodiments, the digital twin ecosystem's biomimetic engine 214 produced a list of gene variants as VUSs 218 potentially associated with the pathophysiology of a targeted disease.

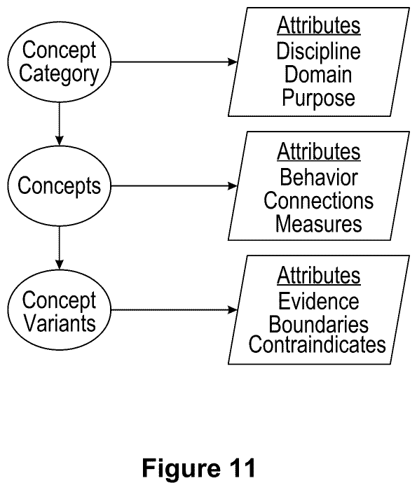

FIG. 11 illustrates a biomimetic digital twin ecosystem including a qualitative metaontology with domains that can be populated and mapped independently by the subject matter experts, in accordance with some embodiments.

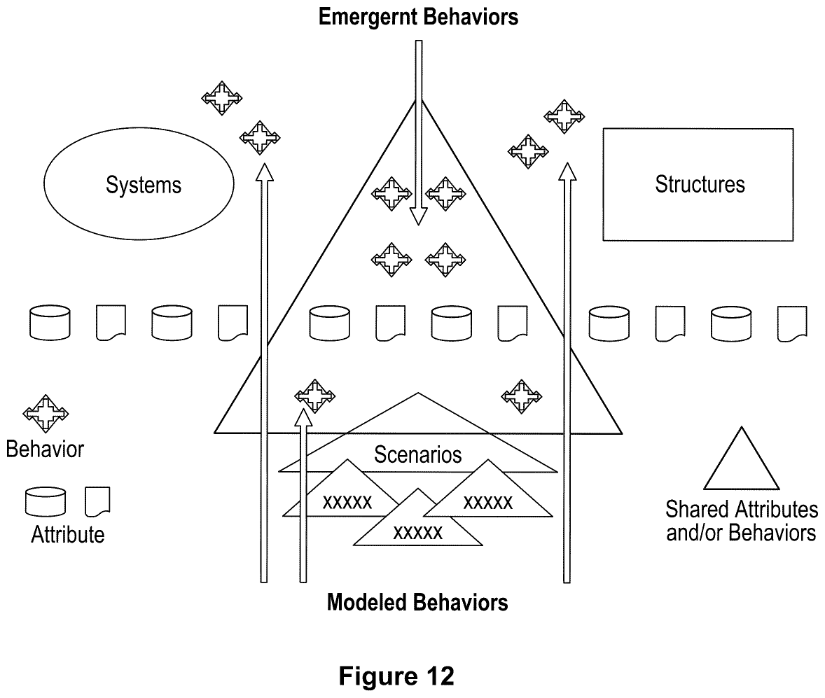

FIG. 12 illustrates a biomimetic digital twin ecosystem model and ecosystem design, in accordance with some embodiments. Models are scoped around known behaviors and designed by imitating (twinning) the understood structures, systems, and scenarios of the modeled behaviors. Emerging behaviors are not predictions, but evidence to be considered by experts.

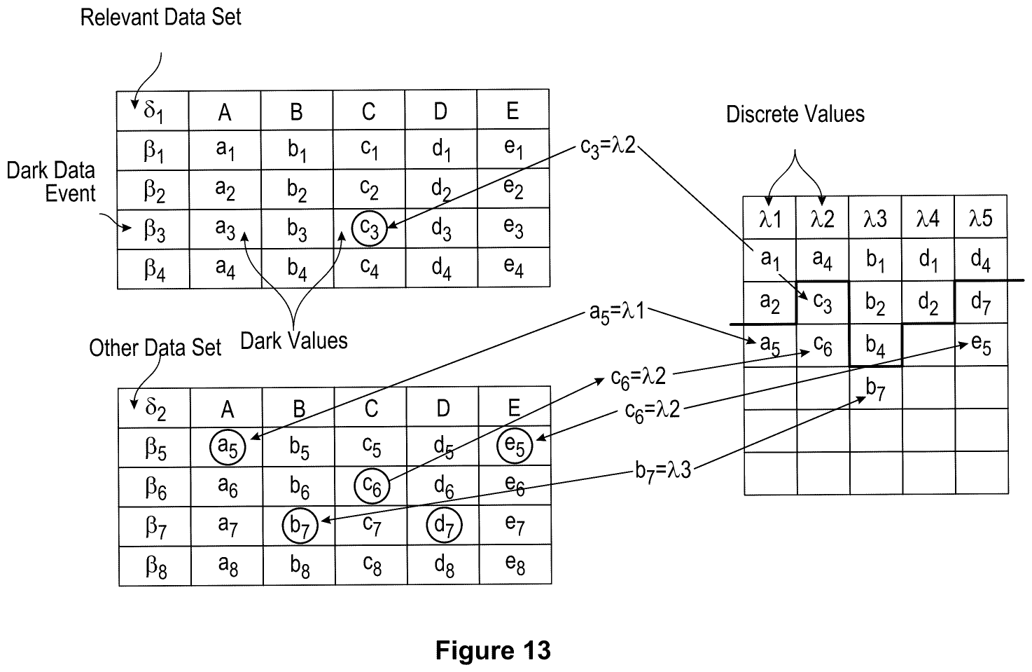

FIG. 13 illustrates an engineering-level view of dark data discovery, in accordance with some embodiments.

The terminology used in the description of the embodiments herein is for the purpose of describing particular embodiments only and is not intended to limit the scope of claims. As used in the description of the embodiments and the appended claims, the singular forms “a,” “an,” and “the” are intended to include the plural forms as well, unless the context clearly indicates otherwise. It will also be understood that the term “and/or” as used herein refers to and encompasses any and possible combinations of one or more of the associated listed items. It will be further understood that the terms “comprises” and/or “comprising,” when used in this specification, specify the presence of stated features, elements, and/or components, but do not preclude the presence or addition of one or more other features, elements, components, and/or groups thereof.

It will also be understood that, although the terms first and second may be used herein to identify various elements, these elements should not be limited by these terms. These terms are only used to distinguish one element from another. For example, a first electrode could be termed a second electrode, and, similarly, a second electrode could be termed a first electrode, without departing from the scope of the embodiments. The first electrode and the second electrode are both electrodes, but they are not the same electrode.

Example 1: Application of Knowledge Engineering Via the Use of a Biomimetic Digital Twin Ecosystem, Phenotype Driven Variant Analysis, and Exome Sequencing to Understand the Molecular Mechanisms of Disease

Applied Artificial Intelligence, particularly Large Language Models, in biomedical research is accelerating, but effective discovery and validation requires a toolset without limitations or bias. In accordance with at least some embodiments disclosed herein is the realization that there is a need to advance the mathematical, statistical, and computational foundations of digital twins in applications across science, medicine, engineering, and society. An innovative method that incorporated phenotype ranking algorithms with knowledge engineering via a biomimetic digital twin ecosystem was developed. This ecosystem applied real-world reasoning principles to non-normalized, raw data to identify hidden or “dark data”. A whole exome sequencing study on patients with endometriosis was performed and four VUSs potentially associated with endometriosis-related disorders were identified in nearly all patients analyzed. One VUS was identified in patient samples 222 and could be a biomarker for diagnostics. This method can be used to understand the mechanisms of any disease, for virtual clinical trials, and to identify effective new therapies.

Artificial intelligence (AI), machine learning (ML), and large language models (LLM) has and continues to transform biomedical research and healthcare. The integration of these into biomedical research holds the promise of enhancing operational efficiency and reducing costs, improving diagnostic ability, uncovering new therapeutic targets, and enabling increasingly personalized medical treatments. Our healthcare future will be defined by how we leverage massive amounts of data for value and efficiency via AI/ML/LLM analysis.

While AI, ML and LLM holds tremendous promise for driving advances in biomedical research, these technologies, like technologies, have limitations. Traditional AI/ML/LLM normalizes data and removes outliers, thus hindering the identification of hidden or “dark” data. In fairness, it must be said that this normalization of data to remove outliers can be argued is to simplify datasets and these can also be adjusted in other ways as well. AI, ML and LLMs also require a test training set to perform the analysis, and this could unintentionally introduce bias into the process. However, in using AI/ML/LLMs, one can modify and enhance training sets to identify the problem more accurately to be solved to reduce bias.

In accordance with at least some embodiments disclosed herein is the realization that the huge datasets required by traditional AI/ML/LLMs, and the associated scale of combinatorial math limit the ability of the algorithms to explore biological complexity, relegating most key relationships and critical interactions into “dark data”-data that is unseen, unexplored and as a result, unanalyzed.

In accordance with at least some embodiments disclosed herein is the realization that there is a need to address these issues and to provide guidance to the biomedical community, e.g., using of biomimetic digital twins' technology to more effectively model multidimensional and multi-scale biological complexity. An important lesson from the long and complex history of neural networks and artificial intelligence is that revolutionary technology can be based on ideas and principles drawn from an understanding of life, rather than on direct harnessing of life's mechanisms or hardware.

Across multiple domains of science, engineering, and medicine, excitement is growing about the potential of digital twins to transform scientific research, industrial practices, and many aspects of daily life. A digital twins couple computational models with a physical counterpart to create a system that is dynamically updated through bidirectional data flows as conditions change. Going beyond traditional simulation and modeling, digital twins could enable improved medical decision-making at the individual patient level, predictions of future weather and climate conditions over longer timescales, and safer, more efficient engineering processes. However, many challenges remain before these applications can be realized.

In accordance with at least some embodiments disclosed herein is the realization that the foundational research and resources needed to support the development of digital twin technologies. In accordance with at least some embodiments disclosed herein is the realization that there is a need to address these issues and to provide guidance to the biomedical community, e.g., the notion that the digital twin virtual representation be fit for purpose, meaning that the virtual representation, e.g., model types, fidelity, resolution, parameterization, and quantities of interest, be chosen, and in many cases dynamically adapted, to fit the decision task and computational constraints at hand, as well as acceptable cost.

Due to the heterogeneity, complexity, multimodality, and breadth of biomedical data, the harmonization, aggregation, and assimilation of data and models to effectively combine these data into biomimetic digital twins require significant technical research.

For many applications, the models that underlie the digital twin virtual representation must represent the behavior of the system across a wide range of spatial and temporal scales. For systems with a wide range of scales on which there are significant nonlinear scale interactions, it may be impossible to represent explicitly in a digital model the full richness of behavior at scales and including interactions.

Technical challenges in modeling, computation, and data pose current barriers to implementing biomimetic digital twins for biomedical use. Because medical data are often sparse and collecting data can be invasive to patients, researchers need strategies to create working models despite missing data.

A combination of data-driven and mechanistic models can be useful to this end, but these approaches can remain limited due to the complexities and lack of understanding of the full biological processes even when sufficient data are available. In addition, data heterogeneity and the difficulty of integrating disparate multimodal data, collected across different time and size scales, also engender significant research questions. The methods as disclosed herein harmonize, aggregate, and assimilate heterogenous data for biomedical/biomimetic digital twins. Furthermore, achieving interoperability and composability of models will be essential. In accordance with at least some embodiments disclosed herein is the realization that there is a need to address gaps and some limits of AI methodologies when it comes to modeling and exploring the biological complexity of the real world. Digital twins represents new theories and methods to address the multi-dimensional, multi-scale characteristics of problems in modeling and advanced analytics in general, and in biomedicine in particular.

AI/ML/LLMs and biomimetic digital twins could very well complement each other if the techniques are used most carefully and with enough knowledge in hands.

To address these issues, in this example, a biomimetic digital twin ecosystem incorporated is into an advanced genomics experimental protocol. It is believed that this is the first report incorporating this methodology into research to understand the pathophysiology of disease.

A study utilizing the biomimetic knowledge engineering methodology were performed for creating an ecosystem of digital twins that implement real-world reasoning principles and that analyze data that is raw and in its original state-meaning that no cleansing or normalization that removes outliers and hides relationships and impacts within data sets are performed. The use of this methodology has both leveraged and utilized dark data and has enabled unexpected discovery.

This example focused on the molecular mechanisms of endometriosis. Endometriosis is an inflammatory condition occurring in 5-10% of women of reproductive age and is associated with debilitating pelvic pain and infertility. It was characterized by the presence of endometrial-like tissue outside the uterus, mainly on pelvic organs. Definitive diagnosis requires visualization of lesions during surgery, contributing to a delay in diagnosis that globally averages seven years from symptom onset. Causes of endometriosis remain largely unknown, but the condition has an estimated heritability of ˜ 50% with ˜26% estimated to be due to common genetic variation in the populations studied.

The molecular mechanisms involved in the development of endometriosis are still being actively researched, and our understanding of the exact processes is evolving. While the precise mechanisms are not fully elucidated, several key molecular factors and pathways are implicated in the pathogenesis of endometriosis. These include the Epithelial-Mesenchymal Transition (EMT), angiogenesis, and vascularization. Chronic inflammation and immune dysregulation are considered important contributors, and hormonal factors also play a significant role. Finally, the role that genetic and epigenetic factors play in the pathogenesis of endometriosis have yet to be comprehensively identified.

Genetic and epigenetic alterations are investigated for their role in endometriosis susceptibility and development. Various genetic polymorphisms and mutations are associated with an increased risk of endometriosis. Epigenetic modifications, including DNA methylation, histone modifications, and microRNA expression changes, can influence gene expression patterns in endometrial cells, affecting processes such as hormone signaling, inflammation, and tissue remodeling. It's important to note that these molecular mechanisms are not mutually exclusive, and they likely interact and influence each other in a complex manner.

Previous multiomic studies such as next generation sequencing to identify pathogenic variants, microarrays to identify polymorphic markers, RNA Seq analysis for gene expression, and epigenetic analysis have provided limiting and confusing results to identify genomic markers associated with the pathogenesis of endometriosis. One group performed a genome-wide association study (GWAS) meta-analysis, which included 60,674 cases and 701,926 controls of European and East Asian descent and identified 42 genome-wide significant loci comprising 49 distinct association signals. While this study was and provided important information on the identification of genomic loci potentially associated with the pathogenesis of endometriosis, one must note that it did not involve a diverse cohort comprised of a wide range of different ethnic populations. Additional research may yield knowledge about the potential role of ethnicity in the mechanism of this disease.

In this example, an innovative approach utilizing whole exome sequencing, phenotype-driven ranking analysis and a biomimetic digital twin ecosystem to identify dark data associated with the molecular profile of endometriosis was developed. Significantly, evidence for a potential biomarker and a chromosomal “hotspot” associated with the pathogenesis of endometriosis has been provided.

In some embodiments, endometriotic and normal matched samples were biopsied in a clinical diagnostic setting.

In some embodiments, whole exome NGS was performed on each sample to determine the presence or absence of known pathogenic mutations, and variants of unknown clinical significance (VUS) associated with endometriosis (FIG. 2B).

Whole genome amplified DNA (50 ng) from each sample was used as input for library preparation. The library prep was done through commercially available NGS library prep service. The DNA sample underwent enzymatic preparation to produce fragment sizes of ˜200 bp. This was followed by ligation using full length adapters. The samples then underwent a purification by beads selectively binding DNA fragments at certain size for cleanup and are washed. A PCR amplification was then performed followed by a second bead cleanup. The samples were then sized and quantitated. Samples were pooled with no more than 12 samples per pool and a 16-hour hybridization was preformed using a panel of oligonucleotide probes.

A bead capture and a set of post-hybridization washes were performed using a hybridization and wash Kit. A post-hybridization amplification using library amplification primers was done, followed by a bead cleanup. The pools were sized and quantitated once more. The pools were normalized and pooled into a single pool.

The pooled libraries were then denatured and loaded onto a sequencing system and sequenced using a sequencing reagent kit. The libraries bound to grafted oligoes on the flow cell and then hybridized and bridge on their specific oligo and undergo multiple cycles of amplification. This forms clusters used a cluster generation technology. Then the clusters underwent 2-channel sequencing by synthesis (SBS) chemistry.

The experimental protocol included a sequencing system for short-read NGS, a secondary analysis, and a tertiary analysis. First, the whole exome sequencing were comprehensively validated against National Institute of Standards and Technology (NIST) reference/validation samples. Accuracy, sensitivity, specificity, positive predictive value, negative predictive value, positive percent agreement, precision (inter- and intra-) assays were performed to complete the validation process.

College of Pathologists (CAP) surveys were utilized. Blinded DNA sequencing to previously known samples was also performed to ensure the accuracy of the results.

Required passing QC metrics for each sample sequenced included, but are not limited to one or more of:
Total_input_reads, which is greater than 49,000,000 in some embodiments;
Number_of_duplicate_marked_reads_pct: under 10%.
Uniformity_of_coverage_pct_gt_02mean_over_target_region: Above 95%
Average_alignment_coverage_over_target_region: Above 85%
Pct_of_target_region_with_coverage_20×_inf: >95%
Short-read sequencing (˜350 bp) is the best modality to use for this study as long-range sequencing (˜3000-5000 bps) is more relevant to identify structural variants. Furthermore, the current standard of care for clinical NGS testing, is using short-read sequencing.

Upon sequencing, a secondary analysis platform were used for secondary analysis. This enrichment is an accurate and efficient end-to-end (FASTQ to VCF) secondary analysis solution for whole exome data. This app took input files in FASTQ, BAM, and CRAM format. Files may be decompressed, go through Map/Align/Sort, and go through variant calling using a variant interpretation platform.

For tertiary analysis, variants, and phenotype ranked variants for each sample using a variant interpretation platform are downloaded. A phenotype-driven ranking filter included a clinical decision support software that accelerates variant interpretation and reporting of germline, hereditary and oncology NGS tests at scale. The phenotype-driven ranking filter computed and combined the relevant information related to the variant of interest and distributes the relevant biological context. The phenotype-driven ranking filter offers the possibility of phenotype driven analysis where the user can submit phenotypes or symptoms of suspected disease or disease under investigation along with the .vcf file of the sample. Based on this info, a phenotype-driven ranking algorithm estimated and ranked genomic variants based on the probability of being the causative one for the disease, symptoms, or the phenotypes under investigation by considering multiple variables such as zygosity, predicted pathogenicity of variant, MOI (mode of inheritance), CADD score, and more variant-centric variables as well as the curated molecular insights.

In some embodiments, knowledge Engineering is using a Biomimetic Engine. Each twin models a discrete component of the analytical scope of the ecosystem. Internal properties and behaviors must be modeled to a level of sufficient comprehensiveness to enable the reactions that are required for the ecosystem to reflect the real world to the scope of its design. Each twin can initiate an interaction with others or respond as prompted. Mitigation of bias is achieved by forming independent design of each twin, abstract knowledge graphs populated without defining specific problems or events, and autonomous interactions between the twins.

This real-world reasoning approach enables the construction of models that integrate highly diverse elements and information sources to enable exploration and discovery to a scope that traditional information architecture cannot accommodate.

In some embodiments, Systems Thinking and Real-world Reasoning (RWR) are required. the National Academies of Science (NAS) also recommends addressing complexity using systems thinking. Key observations include, but are not limited to:
Bottom-up, mechanistic, linear approaches to understanding macro-level behavior are limited when considering complex systems.
Bottom-up, reductionist hypotheses and approaches can lead to a proliferation of parameters; this challenge can potentially be addressed by applying top-down, system-level principles.
Systems thinking can be used to predict macroscopic phenomena while bypassing the need to explicitly unmask the quantitative dynamics operating at the microscopic level.

While knowledge engineering efforts seek to incorporate elements of cognitive science, a key aspect of the innovation strategy is the driving role of a cognitive methodology, which is enabled by biomimetic information architectures. Brain processes are systemic and leverage what neuroscientists label plasticity and sparsity.
Plasticity is the ability to engage diverse combinations of neurons and synapses by relevance to the purpose of the analysis, and to dynamically adapt internal functional architectures.
Sparsity is the ability to identify the minimum data required. The brain can respond to situations that are simultaneously new on multiple dimensions and can even categorize one data point.

The neuronal and synaptic architecture of the brain is an ecosystem, which according to the National Academies of Science contains 100 trillion neurons. Systemic architecture, plasticity and sparsity are core to biological learning, but are NOT like ML algorithms. The biomimetic technologies that enable elements of RWR are:
Expertise Graphs
Neural System Dynamics Digital Twins

Principles of plasticity and sparsity by implementing qualitative expertise graphs and leveraging them for contextual selection of data and methods from the in-memory model library can be imitated. Unlike the deterministic methods to which traditional application engineering is limited of necessity, systemic modeling requires the coexistence of chaotic and stochastic model elements, as well as their ability to dynamically interact with the deterministic elements.

For several years AI has been looked to as the leading pathway to genetic understanding and drug development. However, deep learning (DL) and natural language processing (NLP) has three key challenges that are addressed by the biomimetic digital twin ecosystem methodology presented in this manuscript.

In some embodiments, a biomimetic digital twin ecosystem process 1000 (FIG. 10) includes the following steps:
1. Each patient endometrial DNA sample and matched control underwent exome sequencing, secondary analysis, and tertiary analysis. DNA variants and phenotype ranked variants are exported to the digital twin ecosystem's data lake (data lake).
2. Expert knowledge graphs are produced listing previously reported DNA variants potentially associated with the pathophysiology of endometriosis and are exported to the data lake.
3. Expert knowledge graphs are produced from pathology reports on each endometriosis sample and are exported to the data lake.
4. Expert knowledge graphs are produced from each patient medical record and exported to the data lake.
5. The digital twin ecosystem's biomimetic engine (DT engine) then combined data downloaded from Clinical Insight, including in silico calculations, phenotype ranked references, multifactor correlations to the generated knowledge graphs, and produced a list of gene variants classified as VUSs potentially associated with the pathophysiology of endometriosis.
6. The DT engine ranked VUSs according to the number of times that they are present in patient samples but absent from controls and provided output on genes that mapped to the same chromosome arm.
7. The DT engine's output pinpointed four genes, with DNA variants classified as VUSs, and phenotypes potentially associated with the pathophysiology of endometriosis, endometrial cancer, or an endometrial form of ovarian cancer.
8. The DT output data are uploaded into a database on annotated human genes and a phenotype-dependent variants prioritizer to confirm results. The databases include, but are not limited to:
   a) A searchable, integrative database provides information on annotated and predicted human genes. The knowledgebase automatically integrates gene-centric data from ˜150 web sources, including genomic, transcriptomic, proteomic, genetic, clinical, and functional information.
   b) A phenotype-dependent DNA variant/gene prioritizer can identify causal DNA variants with phenotypes. The prioritizer provides search and scoring capabilities, proficiently matching DNA variant-containing genes to submitted disease/symptom/phenotype keywords. The prioritizer algorithm infers direct as well as indirect links between genes and phenotypes.
9. The DT engine does not make recommendations or draw conclusions, but rather provides researchers with evidence for consideration which is not visible to AI or traditional bioinformatics platforms or approaches.

In some embodiments associated with statistical analysis, while statistical methods may ordinarily be applied to the data at this stage in the analysis, the presently disclosed methodology enables researchers to discover real-world evidence that they cannot find using standard research software, including ML/AI tools. Assessing the statistical significance of the evidence, if desired, can be performed but the calculations depend on the researcher's hypotheses in combination with other available evidence. The use of p-values and associated methods are not without controversy. The presently disclosed approach delivers the results and the supporting evidence and adding a statistical component to the outcome could reduce the clarity of the results and possibly add bias.

In some embodiments, DNA variants from variant interpretation platform from each patient's endometriotic and control tissue sample pairs were downloaded. Pathogenic mutations in 8/12 patient samples were identified. These mutations are in the following genes: CPB1 (c.516C>A), CD36 (c.447_450dupTCAA), AXDND1 (c.125dupA), PROKR2 (c.254G>A), PKN1 (c.1663C>A), DUOX2 (c.2654G>T), CHST15 (c.1366dupC), and ATP6V1B1 (c.988G>A).

In some embodiments, these mutations are associated with endometriosis, endometrial cancer, or an endometrial form of ovarian cancer. No pathogenic mutations were identified in normal patient-matched control samples.

In some embodiments, the mutation is CPB1 (c.516C>A). This mutation is found in some patients with ovarian cancer in the analysis of germline and somatic variants in ovarian cancer.

In some embodiments, the mutation is CD36 (c.447_450dupTCAA). Downregulation of CD36 results in reduced phagocytic ability of peritoneal macrophages of women with endometriosis.

In some embodiments, the mutation is AXDND1 (c.125dupA). Mutations in this gene are associated with ovarian cancer.

In some embodiments, the mutation is PROKR2 (c.254G>A). This mutation is associated with Kallmann syndrome. Kallmann syndrome combines an impaired sense of smell with a hormonal disorder that delays or prevents puberty. This hormonal disorder is due to the underdevelopment of specific neurons, or nerves, in the brain that signal the hypothalamus.

In some embodiments, the mutation is PKN1 (c.1663C>A). This mutation is associated in the development of cancer in populations of women studied with severe endometriosis.

In some embodiments, the mutation is DUOX2 (c.2654G>T). Hypoxia-inhibited dual-specificity phosphatase-2 expression in endometriotic cells regulates cyclooxygenase-2 expression and is thought to be associated with the development of endometriotic lesions.

In some embodiments, the mutation is CHST15 (c.1366dupC). CHST15 expression in tissue is thought to be a prognostic factor of tumor cancer antigens in patients with endometrial cancer.

In some embodiments, the mutation is ATP6V1B1 (c.988G>A). This mutation is associated with epithelial ovarian cancer.

In some embodiments, the phenotype ranking was combined with biomimetic digital twin analysis. In some embodiments, phenotype ranked variants using specific key terms that described the phenotype of endometriosis, endometrial cancer or an endometrial form of ovarian cancer using a phenotype-driven ranking filter for each patient sample and matched controls were downloaded. The data into the biomimetic digital twin ecosystem were then exported. Hidden or “dark data” for DNA variants in four genes classified as variants of unknown clinical significance (VUS) in patient samples but not found in patient matched controls were identified.

In some embodiments, the VUSs Identified were in genes MUC20 (12/12), USP17L1 (8/12), FAM66B (8/12), and DEFB109B (12/12).

In some embodiments, the VUS SNP is within the MUC20 gene. MUC20 polymorphisms, especially rs10794288 and rs10902088, are associated with endometriosis as well as endometriosis-related infertility.

In some embodiments, the VUS is within USP17L1—This is predicted to enable cysteine-type endopeptidase activity and thiol-dependent deubiquitinase. Predicted to be involved in protein deubiquitination and regulation of apoptotic process. Atypical regulation of apoptosis could be involved in the pathophysiology of endometriosis.

In some embodiments, the VUS is within FAM66B. This is a long non-coding RNA (lncRNAs), from the family of regulatory ncRNAs. Mechanistic studies indicate that lncRNAs may regulate genes involved in endometriosis by acting as a molecular sponge for miRNAs, by directly targeting regulatory elements via interactions with chromatin or transcription factors or by affecting signaling pathways.

In some embodiments, the VUS is within DEFB109B. Understanding the biology of endometrial stem cell populations is important for defining normal and abnormal endometrial tissue regeneration and lineage cell commitment. This gene plays a role in the transmission of abnormalities across cell lineages and contributes to proliferative disorders, such as endometrial polyps, endometriosis, and endometrial hyperplasia/cancer.

This is the first study incorporating a phenotype-driven ranking filters with knowledge engineering via the use of a biomimetic digital twin ecosystem and genomic analysis, to provide greater understanding of the molecular mechanism of disease.

In some embodiments, 8 pathogenic mutations were identified associated with the pathophysiology of endometriosis, endometrial cancer, or an endometrial form of ovarian cancer, in 8/12 patient samples analyzed. Additionally, the variant interpretation system and the biomimetic digital twin ecosystem identified four VUSs also associated with the development of endometriosis-related disorders.

In some embodiments, one VUS, in the MUC20 gene, maps to chromosome 3, and was identified in all patient samples analyzed. MUC20 polymorphisms, especially rs10794288 and rs10902088, are associated with endometriosis as well as endometriosis-related infertility.

In some embodiments, the other identified VUSs were in genes USP17L1, FAM66B, and DEFB109B mapped to the short arm of chromosome 8.

USP17L1 is predicted to enable cysteine-type endopeptidase activity and thiol-dependent deubiquitinase. This gene is involved in protein deubiquitination and the regulation of the apoptotic process. Atypical regulation of apoptosis is hypothesized to be involved in the pathophysiology of endometriosis.

FAM66B is a long non-coding RNAs (lncRNA), a type of regulatory ncRNA. Mechanistic studies indicate that lncRNAs may regulate genes involved in endometriosis by acting as a molecular sponge for miRNAs, by directly targeting regulatory elements via interactions with chromatin or transcription factors or by affecting signaling pathways.

DEFB109B is involved in normal and abnormal endometrial tissue regeneration and lineage cell commitment. Transmission of abnormalities across cell lineages may contribute to proliferative disorders, such as endometrial polyps, endometriosis, and endometrial hyperplasia/cancer.

In some embodiments, a potential biomarker associated with endometriosis was identified. In some embodiments, the presence of a VUS within the MUC20 gene in patient samples has been demonstrated.

Furthermore, expression of the DEFB109B and FAM66B genes are regulated by the same gene enhancer sequence. These sequences are regulatory DNA sequences that, when bound by transcription factors, enhance the transcription of an associated gene(s). This suggests a possible hotspot on the short arm of chromosome 8 that could be associated with the molecular pathophysiology of endometriosis.

In some embodiments, the functional descriptions, category, and Functionality annotation score obtained from a database annotating and predicting human genes for the identified pathogenic mutations and VUSs are provided in Table 1.

Functionality annotation score allows a quantitative assessment of a gene's annotation status by exploiting the gene's information in the database. The degree of accumulated knowledge for a given gene measured by Functionality annotation score was correlated with the number of publications for a gene, and with the seniority of this entry in the HUGO Gene Nomenclature Committee (HGNC) database.

TABLE 1
Pathogenic mutations and VUSs identified associated
with the pathophysiology of Endometriosis.

| | | | Functionality |
|---|---|---|---|
| | | | annotation |
| Symbol | Description | Category | score |
| CPB1 | Carboxypeptidase B1 | Protein Coding | 45 |
| CD36 | CD36 Molecule | Protein Coding | 53 |
| AXDND1 | Axonemal Dynein Light Chain Domain | Protein Coding | 36 |
| | Containing 1 | | |
| PROKR2 | Prokineticin Receptor 2 | Protein Coding | 46 |
| PKN1 | Protein Kinase N1 | Protein Coding | 51 |
| DUOX2 | Dual Oxidase 2 | Protein Coding | 47 |
| CHST15 | Carbohydrate Sulfotransferase 15 | Protein Coding | 44 |
| ATP6V1B1 | ATPase H+ Transporting V1 Subunit B1 | Protein Coding | 48 |
| USP17L1 | Ubiquitin Specific Peptidase 17 Like Family | Protein Coding | 23 |
| | Member 1 | | |
| FAM66B | Family With Sequence Similarity 66 Member B | RNA Gene | 17 |
| DEFB109B | Defensin Beta 109B | Protein Coding | 16 |
| MUC20 | Mucin 20, Cell Surface Associated | Protein Coding | 41 |

In addition to the biomimetic digital twin engine, a database on annotated human genes and a phenotype-dependent variants prioritizer were incorporated into the analysis. Pathogenic mutations directly related to the pathophysiology of endometriosis are shown in Table 2, and pathogenic mutations and VUSs identified as the mutations indirectly related to the development of endometriosis are shown in Table 3.

TABLE 2
Pathogenic mutations directly associated with the pathophysiology of Endometriosis.

| | | | | | | Global | | | |
|---|---|---|---|---|---|---|---|---|---|
| | | | | | | Rank | | | Average |
| | | | Functionality | | Matched | (Total | | | Disease- |
| | | | annotation | Matched | Phenotypes | Genes | | | Causing |
| Symbol | Description | Category | score | Phenotypes | Scount | 2505) | −LOG10(P) | Score | Likelihood |
| CD36 | CD36 Molecule | Protein Coding | 53 | “Endometriosis” | 1 | 750 | 1.63 | 0.92 | 20.40% |

TABLE 3
Pathogenic mutations and VUSs indirectly associated
with the pathophysiology of Endometriosis.

| | | | | Functionality | |
|---|---|---|---|---|---|
| Implicated | Implicating | | | annotation | Matched |
| Symbol | Symbol | Description | Category | score | Phenotypes |
| MUC20 | MUC16 | Mucin 16, Cell | Protein | 42 | “Endometriosis” |
| | | Surface Associated | Coding | | |
| MUC20 | HGF | Hepatocyte Growth | Protein | 55 | “Endometriosis” |
| | | Factor | Coding | | |
| MUC20 | MUC4 | Mucin 4, Cell | Protein | 44 | “Endometriosis” |
| | | Surface Associated | Coding | | |
| MUC20 | MET | MET Proto- | Protein | 58 | “Endometriosis” |
| | | Oncogene, Receptor | Coding | | |
| | | Tyrosine Kinase | | | |
| MUC20 | TP53 | Tumor Protein P53 | Protein | 57 | “Endometriosis” |
| | | | Coding | | |
| PROKR2 | GNRH1 | Gonadotropin | Protein | 44 | “Endometriosis” |
| | | Releasing | Coding | | |
| | | Hormone 1 | | | |
| PROKR2 | EMSLR | E2F1 MRNA | RNA | 14 | “Endometriosis” |
| | | Stabilizing LncRNA | Gene | | |
| PROKR2 | WNT4 | Wnt Family | Protein | 51 | “Endometriosis” |
| | | Member 4 | Coding | | |
| PROKR2 | GNRHR | Gonadotropin | Protein | 51 | “Endometriosis” |
| | | Releasing Hormone | Coding | | |
| | | Receptor | | | |
| PROKR2 | FGFR1 | Fibroblast Growth | Protein | 58 | “Endometriosis” |
| | | Factor Receptor 1 | Coding | | |
| DUOX2 | ESR1 | Estrogen Receptor 1 | Protein | 57 | “Endometriosis” |
| | | | Coding | | |
| DUOX2 | TP53 | Tumor Protein P53 | Protein | 57 | “Endometriosis” |
| | | | Coding | | |
| DUOX2 | PTGS2 | Prostaglandin- | Protein | 53 | “Endometriosis” |
| | | Endoperoxide | Coding | | |
| | | Synthase 2 | | | |
| DUOX2 | IFNG | Interferon Gamma | Protein | 54 | “Endometriosis” |
| | | | Coding | | |
| DUOX2 | WNT4 | Wnt Family Member | Protein | 51 | “Endometriosis” |
| | | 4 | Coding | | |
| PKN1 | AKT1 | AKT | Protein | 57 | “Endometriosis” |
| | | Serine/Threonine | Coding | | |
| | | Kinase 1 | | | |
| PKN1 | ESR1 | Estrogen Receptor 1 | Protein | 57 | “Endometriosis” |
| | | | Coding | | |
| PKN1 | RELA | RELA Proto- | Protein | 54 | “Endometriosis” |
| | | Oncogene, NF-KB | Coding | | |
| | | Subunit | | | |
| PKN1 | TP53 | Tumor Protein P53 | Protein | 57 | “Endometriosis” |
| | | | Coding | | |
| PKN1 | EGFR | Epidermal Growth | Protein | 58 | “Endometriosis” |
| | | Factor Receptor | Coding | | |
| ATP6V1B1 | IL6 | Interleukin 6 | Protein | 54 | “Endometriosis” |
| | | | Coding | | |
| ATP6V1B1 | WNT4 | Wnt Family Member | Protein | 51 | “Endometriosis” |
| | | 4 | Coding | | |
| ATP6V1B1 | VEGFA | Vascular Endothelial | Protein | 53 | “Endometriosis” |
| | | Growth Factor A | Coding | | |
| ATP6V1B1 | PGR | Progesterone | Protein | 52 | “Endometriosis” |
| | | Receptor | Coding | | |
| ATP6V1B1 | ESR1 | Estrogen Receptor 1 | Protein | 57 | “Endometriosis” |
| | | | Coding | | |
| CHST15 | ESR1 | Estrogen Receptor 1 | Protein | 57 | “Endometriosis” |
| | | | Coding | | |
| CHST15 | HOTAIR | HOX Transcript | RNA | 25 | “Endometriosis” |
| | | Antisense RNA | Gene | | |
| CHST15 | CYP19A1 | Cytochrome P450 | Protein | 52 | “Endometriosis” |
| | | Family 19 Subfamily | Coding | | |
| | | A Member 1 | | | |
| CHST15 | CDKN2A | Cyclin Dependent | Protein | 54 | “Endometriosis” |
| | | Kinase Inhibitor 2A | Coding | | |
| CHST15 | GSTM1 | Glutathione S- | Protein | 46 | “Endometriosis” |
| | | Transferase Mu 1 | Coding | | |
| CPB1 | CPB2 | Carboxypeptidase B2 | Protein | 48 | “Endometriosis” |
| | | | Coding | | |
| CPB1 | PTEN | Phosphatase And | Protein | 55 | “Endometriosis” |
| | | Tensin Homolog | Coding | | |
| CPB1 | IL10 | Interleukin 10 | Protein | 51 | “Endometriosis” |
| | | | Coding | | |
| CPB1 | CDKN2A | Cyclin Dependent | Protein | 54 | “Endometriosis” |
| | | Kinase Inhibitor 2A | Coding | | |
| CPB1 | PGR | Progesterone | Protein | 52 | “Endometriosis” |
| | | Receptor | Coding | | |
| USP17L1 | ESR1 | Estrogen Receptor 1 | Protein | 57 | “Endometriosis” |
| | | | Coding | | |
| USP17L1 | VEGFA | Vascular Endothelial | Protein | 53 | “Endometriosis” |
| | | Growth Factor A | Coding | | |
| USP17L1 | USP17L2 | Ubiquitin Specific | Protein | 32 | “Endometriosis” |
| | | Peptidase 17 Like | Coding | | |
| | | Family Member 2 | | | |
| USP17L1 | PGR | Progesterone | Protein | 52 | “Endometriosis” |
| | | Receptor | Coding | | |
| USP17L1 | TP53 | Tumor Protein P53 | Protein | 57 | “Endometriosis” |
| | | | Coding | | |
| AXDND1 | CYP17A1 | Cytochrome P450 | Protein | 52 | “Endometriosis” |
| | | Family 17 Subfamily | Coding | | |
| | | A Member 1 | | | |
| AXDND1 | CYP1B1 | Cytochrome P450 | Protein | 51 | “Endometriosis” |
| | | Family 1 Subfamily | Coding | | |
| | | B Member 1 | | | |
| AXDND1 | BMP6 | Bone Morphogenetic | Protein | 48 | “Endometriosis” |
| | | Protein 6 | Coding | | |
| AXDND1 | GHRL | Ghrelin And | Protein | 45 | “Endometriosis” |
| | | Obestatin | Coding | | |
| | | Prepropeptide | | | |
| AXDND1 | IL32 | Interleukin 32 | Protein | 42 | “Endometriosis” |
| | | | Coding | | |
| DEFB109B | IQCH | IQ Motif | Protein | 36 | “Endometriosis” |
| | | Containing H | Coding | | |
| DEFB109B | SGPP2 | Sphingosine-1- | Protein | 38 | “Endometriosis” |
| | | Phosphate | Coding | | |
| | | Phosphatase 2 | | | |

| | | Global | | | | |
|---|---|---|---|---|---|---|
| | | Rank | | Average | | Average |
| | Matched | (Total | | Disease- | | Disease |
| Implicated | Phenotypes | Genes | Score | Causing | Score | Causing |
| Symbol | Count | 2505) | (Implicated) | Likelihood | (Implicating) | Likelihood1 |
| MUC20 | 1 | 48 | 0.59 | 0.00% | 0.37 | 0.04% |
| MUC20 | 1 | 83 | 0.59 | 0.00% | 0.26 | 70.64% |
| MUC20 | 1 | 395 | 0.59 | 0.00% | 0.21 | 0.00% |
| MUC20 | 1 | 235 | 0.59 | 0.00% | 0.2 | 56.48% |
| MUC20 | 1 | 35 | 0.59 | 0.00% | 0.12 | 72.85% |
| PROKR2 | 1 | 37 | 0.45 | 45.03% | 0.24 | 27.92% |
| PROKR2 | 1 | 30 | 0.45 | 45.03% | 0.24 | 0.00% |
| PROKR2 | 1 | 4 | 0.45 | 45.03% | 0.22 | 78.03% |
| PROKR2 | 1 | 163 | 0.45 | 45.03% | 0.2 | 60.64% |
| PROKR2 | 1 | 293 | 0.45 | 45.03% | 0.18 | 76.25% |
| DUOX2 | 1 | 26 | 0.44 | 11.15% | 0.27 | 68.53% |
| DUOX2 | 1 | 35 | 0.44 | 11.15% | 0.21 | 72.85% |
| DUOX2 | 1 | 36 | 0.44 | 11.15% | 0.18 | 66.54% |
| DUOX2 | 1 | 58 | 0.44 | 11.15% | 0.13 | 81.00% |
| DUOX2 | 1 | 4 | 0.44 | 11.15% | 0.13 | 78.03% |
| PKN1 | 1 | 152 | 0.31 | 25.43% | 0.18 | 90.77% |
| PKN1 | 1 | 26 | 0.31 | 25.43% | 0.15 | 68.53% |
| PKN1 | 1 | 192 | 0.31 | 25.43% | 0.12 | 80.67% |
| PKN1 | 1 | 35 | 0.31 | 25.43% | 0.12 | 72.85% |
| PKN1 | 1 | 136 | 0.31 | 25.43% | 0.11 | 77.60% |
| ATP6V1B1 | 1 | 31 | 0.19 | 40.73% | 0.11 | 56.07% |
| ATP6V1B1 | 1 | 4 | 0.19 | 40.73% | 0.09 | 78.03% |
| ATP6V1B1 | 1 | 27 | 0.19 | 40.73% | 0.08 | 64.83% |
| ATP6V1B1 | 1 | 25 | 0.19 | 40.73% | 0.07 | 37.86% |
| ATP6V1B1 | 1 | 26 | 0.19 | 40.73% | 0.06 | 68.53% |
| CHST15 | 1 | 26 | 0.18 | 53.63% | 0.11 | 68.53% |
| CHST15 | 1 | 256 | 0.18 | 53.63% | 0.08 | 0.00% |
| CHST15 | 1 | 24 | 0.18 | 53.63% | 0.06 | 49.30% |
| CHST15 | 1 | 70 | 0.18 | 53.63% | 0.04 | 47.77% |
| CHST15 | 1 | 33 | 0.18 | 53.63% | 0.04 | 0.00% |
| CPB1 | 1 | 719 | 0.16 | 41.72% | 0.09 | 40.67% |
| CPB1 | 1 | 44 | 0.16 | 41.72% | 0.08 | 78.93% |
| CPB1 | 1 | 39 | 0.16 | 41.72% | 0.07 | 63.14% |
| CPB1 | 1 | 70 | 0.16 | 41.72% | 0.07 | 47.77% |
| CPB1 | 1 | 25 | 0.16 | 41.72% | 0.06 | 37.86% |
| USP17L1 | 1 | 26 | 0.15 | 0.00% | 0.08 | 68.53% |
| USP17L1 | 1 | 27 | 0.15 | 0.00% | 0.07 | 64.83% |
| USP17L1 | 1 | 821 | 0.15 | 0.00% | 0.07 | 4.21% |
| USP17L1 | 1 | 25 | 0.15 | 0.00% | 0.06 | 37.86% |
| USP17L1 | 1 | 35 | 0.15 | 0.00% | 0.06 | 72.85% |
| AXDND1 | 1 | 45 | 0.07 | 31.43% | 0.05 | 90.83% |
| AXDND1 | 1 | 105 | 0.07 | 31.43% | 0.03 | 16.89% |
| AXDND1 | 1 | 184 | 0.07 | 31.43% | 0.03 | 19.17% |
| AXDND1 | 1 | 241 | 0.07 | 31.43% | 0.03 | 21.61% |
| AXDND1 | 1 | 239 | 0.07 | 31.43% | 0.02 | 36.14% |
| DEFB109B | 1 | 337 | 0.03 | 0.00% | 0.02 | 69.17% |
| DEFB109B | 1 | 2257 | 0.03 | 0.00% | 0.01 | 68.13% |

In some embodiments, the digital twin ecosystem can uncover additional variants classified as VUSs on genes potentially associated with endometriosis.

These results provide evidence that the VUSs are highly likely to play some role in the pathophysiology of endometriosis, endometrial cancer, and an endometrial form of ovarian cancer. Furthermore, an innovative way to potentially re-classify VUSs is described.

For several years traditional AI has been looked to as the leading pathway to genomic understanding and drug development. Deep learning (DL) and natural language processing (NLP) have three key challenges that are addressed by the biomimetic digital twin ecosystem methodology. These challenges include instability, blindness to dark data, and risks and biases that are being challenged by some in the AI community.

Addressing each of the three above limitations requires finding and connecting biological dark data. Standard AI is blind to the dark data because algorithms can only find what they are engineered to find. Furthermore, AI can only work within the narrow limits of the algorithm's training data, which is large (many rows), narrow (limited attributes) and cleansed (outliers removed). These factors greatly diminish one's ability to identify high-value insights.

In some implementations, shedding light on the darkened insights requires small/wide data methods. Wide data allows analysts to examine and combine a variety of small and large attributes from diverse sources, while small data is focused on applying analytical techniques that look for useful information within limited sets of data. Hence, the outputs of the digital twin ecosystem are arrays of scenarios with associated evidence, and AI/ML/LLMs outputs predictions. These outputs should be, in the future, compared closely. The comparison results will be valuable in identifying additional gaps in biomedical research.

A Biomimetic Digital Twin Ecosystem is very powerful in its ability to analyze small, wide data sets and identify dark data. Additional studies are required to discover the role of ethnicity, if any, in the pathogenesis of endometrial-related disorders.

In conclusion, using the advanced genomic process, including whole exome sequencing, a clinical information application, and a biomimetic digital twin ecosystem, it has been demonstrated how researchers can more clearly define the molecular mechanism of disease. The analysis identified a potential biomarker for a molecular test to determine whether a patient has endometriosis. These data also identified a potential hotspot for the molecular study of endometriosis on the short arm of chromosome 8. The combination of a knowledge engineering platform and molecular analyses can be used for the identification of molecular mechanisms for any disease. It can include and clarify the role of ethnicities in the severity of disease, perform virtual clinical trials, and aid in the rapid identification of new therapies for the effective treatment of disease.

### Example 2: Biomimetic Digital Twins and Multiomics: Applications to Rheumatoid Arthritis and the Potential Reclassification of Variants of Unknown Clinical Significance Key Points

Biomimetic digital twins have been incorporated with the multiomics platform to potentially clarify the pathogenesis of a complex disorder, rheumatoid arthritis. It was accomplished by identifying dark data—complex relationships that are not visible in bioinformatics platforms—as either directly or indirectly related to the development of rheumatoid arthritis.

These results suggested that by using biomimetic digital twins and a Multiomics platform, the process of reclassifying variants of unknown clinical significance (VUS) can be started.

The reclassification of VUSs would play a critical role in complex diagnostics and drug development.

The National Academies of Science issued a report on 12.15.23 “Foundational Research Gaps and Future Directions for Digital Twins”. This report described the importance of using biomimetic digital twins and multiomics in research. These are incorporated in the analysis of patients with rheumatoid arthritis (RA). Exome sequencing, genotype-phenotype ranking, and digital twin analysis are performed. Five pathogenic, and one likely pathogenic DNA variants in patient samples analyzed but absent from controls, were identified. The variants identified in these genes, P2RX7, HTRA2, PTPN22, FLG, CD46, and EIF4G1 play a role in the development of RA. 3172 variants of unknown clinical significance (VUSs) in patient samples, absent from controls, were also identified. VUSs appeared to be associated with RA. Hidden or “dark data” from six genes were identified. The genes often found in patient samples included genes HIF1A, HLA-DOA, PTGER3, HIPK3, TGFBR3 and HIF1A-AS3. VUSs identified in genes HIF1A, HLA-DOA, PTGER3, HIPK3 are directly related to the pathogenesis of RA, whereas VUSs identified in genes TGFBR3 and HIF1A-AS3 are indirectly related to the pathophysiology of RA. These results suggested that by using digital twins and multiomics, more insight into the development of RA can be gained. Potentially the process of reclassifying VUSs can be started. The reclassification of VUSs will play a critical role in complex molecular diagnostics and drug development.

Modeling real-world complexity has long been the goal of biomedical methodology and software, but it persists in being a major challenge due to the astronomical combinatorial possibilities of biological systems which are multidimensional and multiscale. Modeling the interactions within molecular genomic ecosystems and how they interact with human biology has long been the driver of biomedical research and technological advancement. One aim has been to understand the relationships between genotypes and phenotypes by correct classification of DNA variants, but the results thus far are limited due to variants of unknown clinical significance (VUS).

The futility of trying to model real world complexity by scaling data volume and processing power has been identified and communicated for nearly two decades but has not been adequately assimilated.

In accordance with at least some embodiments disclosed herein is the realization that across multiple domains of science, engineering, and medicine, excitement is growing about the potential of digital twins to transform scientific research, industrial practices, and many aspects of daily life. A digital twin combines computational models with a physical counterpart to create a system that is dynamically updated through bidirectional data flows as conditions change. Going beyond traditional simulation and modeling, digital twins could enable improved precision medicine and healthcare by more clearly understanding the pathophysiology of disease. In accordance with at least some embodiments disclosed herein is the realization that the foundational research and resources needed to support the development of digital twin technologies is ongoing. Biomimetic digital twins apply many of the recommendations from the report, including the conclusion that new theories and methods are required to address the multi-dimensional, multi-scale characteristics of problems in modeling and advanced analytics in general, and in biomedicine in particular.

In some embodiments, a biomimetic digital twin ecosystem is incorporated into advanced multiomics experimental protocols. The biomimetic knowledge engineering methodology are utilized for creating an ecosystem of digital twins that implement real-world reasoning principles and that analyzed data that is raw and in its original state-meaning that no cleansing or normalization that removed outliers and hide relationships and impacts within data sets are performed. The use of this methodology has both leveraged and utilized dark data and has enabled unexpected discovery. Evidence for a potential biomarker for a less invasive diagnostic for endometriosis are provided and a chromosomal “hotspot” associated with the pathogenesis of endometriosis is identified. It has been demonstrated that using multiomics and digital twins how researchers can more clearly define the molecular mechanism of disease.

This example focused on the molecular mechanisms of rheumatoid arthritis (RA). RA is a multifactorial autoimmune disease of unknown etiology, primarily affecting the joints, then extra-articular manifestations can occur. RA causes joint inflammation, which in severe cases may result in permanent joint damage and disability. Additionally, RA may affect other organs, including the lungs, heart, blood vessels, skin, and eyes. Rheumatoid arthritis affects approximately 1 of every 200 adults worldwide and occurs 2 to 3 times more frequently in women than men. It can affect people of any age, but peak onset is from age 50 to 59 years.

Most epidemiological studies in rheumatoid arthritis are conducted in western countries, showing an RA prevalence in the range of 0.5-1.0% in the United States. The cumulative lifetime risk of developing adult-onset RA has been roughly estimated at 3.6% for women and 1.7% for men.

RA has a strong genetic component. Twin studies have estimated the heritability of RA to be approximately 60%. This number is observed in anti-cyclic citrullinated peptide antibody (ACPA) positive patients. ACPA-positive patients are considered to have a more severe subset of RA, with more severe joint destruction and a higher mortality rate. ACPA positivity is also associated with older age, female gender, smoking, joint complaints, and first-degree relatives with RA.

The disease concordance of identical twins is 12-15%, indicating that environmental factors also play an important role in susceptibility.

Genotype-phenotype relationships have identified 6786 genes with some potential association to the pathophysiology of RA.

Other studies have suggested that over 100 loci are identified across genomes harboring RA susceptibility variants by genome-wide association studies, with fine mapping, candidate gene approaches, and a meta-analysis of genome-wide association studies involving >100,000 individuals.

RA is a complex multifactorial disease with both genetic and environmental risk factors contributing to RA, and multiple risk factors may be required before the threshold at which RA is triggered.

In this example, the research using exome sequencing, phenotype-driven ranking analysis, a biomimetic digital twin analysis, a database on annotated human genes, and a phenotype-dependent variants prioritizer were utilized to identify dark data associated with the molecular profile of the complex multifactorial disorder, rheumatoid arthritis. Significantly, additional results demonstrating that digital twins and multiomics can play a role in the clarification in the pathophysiology of a complex genetic disorder are provided. Furthermore, the potential role of using the platform in identifying variants of unknown clinical significance (VUSs) potentially associated with the development of rheumatoid arthritis have been demonstrated and it suggested a way to potentially reclassify VUSs as pathogenic, likely pathogenic, benign, or likely benign.

In some embodiments, patient samples analyzed in this study were from immunodeficiency whole exome clinical tests ordered by physicians with a signed informed consent. The patient ages ranged from 65 to 72, nineteen are of Caucasian ethnicity, four are African American, and two are of Asian descent. Thirteen samples are from female sex and twelve from male sex.

Whole exome NGS was performed on each sample to determine the presence or absence of known pathogenic, or likely pathogenic mutations, and variants of unknown clinical significance (VUS) associated with rheumatoid arthritis (FIG. 2B).

In some embodiments, whole-genome amplified DNA (e.g., 50 ng) from each sample was used as input for library preparation. The library prep was done is done through commercially available NGS library prep service. The DNA sample underwent enzymatic preparation to produce fragment sizes of ˜200 bp. This was followed by ligation using full length adapters. The samples then underwent a purification by beads selectively binding DNA fragments at certain size cleanup and are washed. A PCR amplification was then performed followed by a seco bead cleanup. The samples were then sized and quantitated. Samples were pooled with no more than 12 samples per pool and a 16-hour hybridization was preformed using a panel of oligonucleotide probes.

A bead capture and a set of post-hybridization washes were performed using a hybridization and wash Kit. A post-hybridization amplification using library amplification primers was done, followed by a bead cleanup. The pools were sized and quantitated once more. The pools are normalized and pooled into a single pool.

The pooled libraries were then denatured and loaded onto a sequencing system and sequenced using a sequencing reagent kit. The libraries bound to grafted oligoes on the flow cell and then hybridized and bridge on their specific oligo and undergo multiple cycles of amplification. This forms clusters using a cluster generation technology. Then the clusters underwent 2-channel sequencing by synthesis (SBS) chemistry.

The experimental protocol included a sequencing system for short-read NGS, a secondary analysis, and a tertiary analysis. First, the whole exome sequencing was comprehensively validated against National Institute of Standards and Technology (NIST) reference/validation samples. Accuracy, sensitivity, specificity, positive predictive value, negative predictive value, positive percent agreement, precision (inter- and intra-) assays were performed to complete the validation process.

College of Pathologists (CAP) surveys are utilized. Blinded DNA sequencing to previously known samples were also performed to ensure the accuracy of these results.

Required passing QC metrics for each sample sequenced.

Total_input_reads: Grater than 49,000,000

Number_of_duplicate_marked_reads_pct: under 10%

Uniformity_of_coverage_pct_gt_02mean_over_target_region: Above 95%

Average_alignment_coverage_over_target_region: Above 85%

Pct_of_target_region_with_coverage_20×_inf: >95%

Short-read sequencing (˜350 bp) is the best modality to use for this study as long-range sequencing (˜3000-5000 bps) is more relevant to identify structural variants. Furthermore, the current standard of care for clinical NGS testing, is using short-read sequencing.

Upon sequencing, a secondary analysis platform was used for secondary analysis. This enrichment is an accurate and efficient end-to-end (FASTQ to VCF) secondary analysis solution for whole exome data. This app took input files in FASTQ, BAM, and CRAM format. Files may be decompressed, go through Map/Align/Sort, and go through variant calling using a variant interpretation platform. In some embodiments, the output for the secondary analysis of the whole exome for one of the rheumatoid arthritis (RA) patients are shown in Appendix 1.

For tertiary analysis, variants, and phenotype ranked variants for each sample using a variant interpretation platform were downloaded. A phenotype-driven ranking filter is a clinical decision support software that accelerates variant interpretation and reporting of hereditary and oncology NGS tests at scale. The phenotype-driven ranking filter computed and combined the relevant information related to the variant of interest and distributes the relevant biological context. The phenotype-driven ranking filter offers the possibility of phenotype driven analysis where the user can submit phenotypes or symptoms of suspected disease or disease under investigation along with the .vcf file of the sample. Based on this information, a phenotype-driven ranking algorithm estimated and ranked genomic variants based on the probability of being the causative one for the disease, symptoms, or the phenotypes under investigation by considering multiple variables such as zygosity, predicted pathogenicity of variant, MOI (mode of inheritance), CADD score, and more variant-centric variables as well as the curated molecular insights. In some embodiments, the comparison Between Standard Artificial Intelligence and a Biomimetic Digital Twin Analysis are shown as discussed below.

In some embodiments, a Biomimetic Digital Twin Architecture is innovative in the following aspects: human expertise graphs, model & ecosystem design, real-world data (RWD) approach, dark data discovery, and transparency.

In some embodiment associated with human expertise graphs, experts use qualitative reasoning for problem analysis. Therefore, the biomimetic digital twin ecosystem must include a qualitative metaontology with domains that can be populated and mapped independently by the subject matter experts. FIG. 11 is a high-level example. Experts map relevant attributes in the provided data sources to their own qualitative models of the domains or to industry standard ontologies.

In some embodiments associated with model and ecosystem design, models are scoped around known behaviors and designed by imitating (twinning) the understood structures, systems, and scenarios of the modeled behaviors. Emerging behaviors are not predictions, but evidence to be considered by experts (FIG. 12).

In some embodiment associated with ecosystem architecture, each twin models a discrete component of the analytical scope of the ecosystem. Internal properties and behaviors must be modeled to a level of sufficient comprehensiveness to enable the reactions that are required for the ecosystem to reflect the real world to the scope of its design. Each twin can initiate an interaction with others or respond as prompted. Mitigation of bias is achieved by: independent design of each twin, abstract knowledge graphs populated without defining specific problems or events, and autonomous interactions between the twins.

In some embodiments associated with real-world data (RWD), a data lake is populated with the required small/wide data sources, which could be small data sets such as extracts from patient records or outputs of larger systems such as bioinformatics platforms. Tables are in their native schema without normalization or cleansing-RWD.

Contextualization is the primary method of interpreting data and assessing the relevance of evidence to a defined problem. Big data is more likely to pose challenges rather than help. A recent article titled The Limits of Data in issues.org, from the National Academy of Sciences, concluded “Data is powerful because it's universal. The cost is context.”

In some embodiments associated with dark data discovery, the relevance computation engine leverages the combined expertise graphs to identify multiscale and multidimensional relationships (FIG. 13) across the data sets in the data lake. FIG. 13 shows an engineering-level view of dark data discovery.

In this case multifactor correlations between pathogenic variants (relevant), VUSs (other) and knowledge graphs populated by the researchers produced the reported findings.

In some embodiments, the findings are distinct from AI outputs generated using the same inputs. An AI-based data processing model requires very large training datasets, can provide statistically computes predictions rather than discovering contextual relationships so they cannot deliver RWD but frequently produce hallucinations, and uses black box algorithms that provide no explanation for deriving the outputs.

In some embodiments associated with transparency, the biomimetic digital twin ecosystem is NOT a black box application. The process of computing relevance using expert mappings is transparent and does not perform any data transformation. The outputs are not predictions but discovered relationships with the associated evidence. The outputs identify the source files and attributes for each value that is presented as part of the evidence, so that everything is traceable. The only variable may be differences in experts' views on the significance of the evidence.

In some embodiments associated with knowledge engineering using a biomimetic engine, each twin models a discrete component of the analytical scope of the ecosystem. Internal properties and behaviors must be modeled to a level of sufficient comprehensiveness to enable the reactions that are required for the ecosystem to reflect the real world to the scope of its design. Each twin can initiate an interaction with others or respond as prompted. Mitigation of bias is achieved by independent design of each twin, abstract knowledge graphs populated without defining specific problems or events, and autonomous interactions between the twins. This real-world reasoning approach enables the construction of models that integrate highly diverse elements and information sources to enable exploration and discovery to a scope that traditional information architecture cannot accommodate.

In some embodiments associated with systems thinking and real-world reasoning (RWR), the NAS also recommends addressing complexity using systems thinking. Key observations include, but are not limited to:
Bottom-up, mechanistic, linear approaches to understanding macro-level behavior are limited when considering complex systems.
Bottom-up, reductionist hypotheses and approaches can lead to a proliferation of parameters; this challenge can potentially be addressed by applying top-down, system-level principles.
Systems thinking can be used to predict macroscopic phenomena while bypassing the need to explicitly unmask the quantitative dynamics operating at the microscopic level.

While knowledge engineering efforts seek to incorporate elements of cognitive science, a key aspect of the disclosed innovation strategy is the driving role of a cognitive methodology, which is enabled by biomimetic information architectures. Brain processes are systemic and leverage what neuroscientists label plasticity and sparsity.
Plasticity is the ability to engage diverse combinations of neurons and synapses by relevance to the purpose of the analysis, and to dynamically adapt internal functional architectures.
Sparsity is the ability to identify the minimum data required. The brain can respond to situations that are simultaneously new on multiple dimensions and can even categorize one data point.

The neuronal and synaptic architecture of the brain is an ecosystem, which according to the National Academies of Science contains 100 trillion neurons. Systemic architecture, plasticity and sparsity are core to biological learning, but are NOT like ML algorithms. The biomimetic technologies that enable elements of RWR are:
Expertise Graphs
Neural System Dynamics Digital Twins

Principles of plasticity and sparsity can be imitated by implementing qualitative expertise graphs and leveraging them for contextual selection of data and methods from the in-memory model library. Unlike the deterministic methods to which traditional application engineering is limited of necessity, systemic modeling requires the coexistence of chaotic and stochastic model elements, as well as their ability to dynamically interact with the deterministic elements.

For several years AI has been looked to as the leading pathway to genetic and genomic understanding and drug development. However, deep learning (DL) and natural language processing (NLP) have three key challenges that are addressed by the biomimetic digital twin ecosystem methodology presented in this manuscript.

### Biomimetic Digital Twin Ecosystem Process (DT) Tailored for the Analysis

1) Each patient DNA sample and controls underwent exome sequencing, secondary analysis, and tertiary analysis. DNA variants and phenotype ranked variants are exported to the Digital Twin Ecosystem's data lake (data lake).
2) Expert knowledge graphs are produced listing previously reported DNA variants potentially associated with the pathophysiology of rheumatoid arthritis and are exported to the data lake.
3) Expert knowledge graphs are produced from each patient medical record and exported to the data lake.
4) The Digital Twin Ecosystem's biomimetic engine (DT engine) then combined data downloaded from Clinical Insight, including in silico calculations, phenotype ranked references, multifactor correlations to the generated knowledge graphs, and produced a list of gene variants classified as VUSs potentially associated with the pathophysiology of RA.
5) The DT engine ranked VUSs according to the number of times that they are present in patient samples but absent from controls.
6) The DT engine's output pinpointed six genes, with DNA variants classified as VUSs, and phenotypes potentially associated with the pathophysiology of rheumatoid arthritis.
7) The DT output data are uploaded into a gene database or a gene prioritizer to identify genotype/phenotype relationships associated with pathophysiology of rheumatoid arthritis.
   a. The gene database (e.g., corresponding to a first database 212A in FIG. 2A) may be a searchable, integrative database that provides information on annotated and predicted human genes. The knowledgebase automatically integrates gene-centric data from ˜200 web sources, including genomic, transcriptomic, proteomic, genetic, clinical, and functional information.
   b. The gene prioritizer (e.g., corresponding to a first database 212B in FIG. 2A) may be a phenotype-dependent DNA variant/gene prioritizer, that can identify causal DNA variants with phenotypes. This provides search and scoring capabilities, proficiently matching DNA variant-containing genes to submitted disease/symptom/phenotype keywords. The algorithm infers direct as well as indirect links between genes and phenotypes.

8) The DT engine does not make recommendations or draw conclusions, but rather provides researchers with evidence for consideration which is not visible to AI or traditional bioinformatics platforms or approaches.

In some embodiments associated with statistical analysis, while statistical methods may ordinarily be applied to the data at this stage in the analysis, the presently disclosed methodology enables researchers to discover real-world evidence that they cannot find using standard research software, including ML/AI tools. Assessing the statistical significance of the evidence, if desired, can be performed but the calculations depend on the researcher's hypotheses in combination with other available evidence. The use of p-values and associated methods are not without controversy 20. The presently disclosed approach delivers the results and the supporting evidence and adding a statistical component to the outcome could reduce the clarity of the results and possibly add bias.

In some embodiments, eleven DNA variants from a variant interpretation platform from each patients' sample and controls were downloaded. Five pathogenic and one likely pathogenic DNA variant in 8/25 patient samples analyzed were identified. These mutations are associated with rheumatoid arthritis. These include genes P2RX7, HTRA2, PTPN22 (likely pathogenic), FLG, CD46, and EIF4G1. No pathogenic or likely pathogenic DNA variant is identified in any controls. P2RX7 is a highly expressed receptor on immune cells, triggering the release of cytokines and regulating autoimmune responses. The synthesis of pro-inflammatory cytokines and apoptosis of lymphoid cells can be induced through P2X7. These results suggest a possible involvement of P2X7 in the pathogenesis of inflammatory autoimmune diseases and its role in the development of rheumatoid arthritis. HTRA2 is a serine peptidase that plays a significant role in collagen-induced rheumatoid arthritis. HTRA2 modulates inflammatory responses by controlling TRAF2 stability in a collagen-induced rheumatoid arthritis. PTPN22 encodes of member of the non-receptor class 4 subfamily of the protein-tyrosine phosphatase family. The encoded protein is a lymphoid-specific intracellular phosphatase that associates with the molecular adapter protein CBL and may be involved in regulating CBL function in the T-cell receptor signaling pathway. DNA variants in this gene may be associated with a range of autoimmune disorders including Type 1 Diabetes, rheumatoid arthritis, systemic lupus erythematosus and Graves' disease.

For FLG, antikeratin antibodies and the antiperinuclear factor are the most specific serological markers of rheumatoid arthritis. They are largely the same autoantibodies that recognizes human epidermal filaggrins and profilaggrin-related proteins of buccal epithelial cells (collectively referred to as (pro) filaggrin). The protein encoded by CD46 is a type I membrane protein and is a regulatory part of the complement system. CD46 acts as a cofactor for complement factor I, a serine protease which protects autologous cells against complement-mediated injury by cleaving C3b and C4b deposited on host tissue. CD46 acts as a costimulatory factor for T-cells which induces the differentiation of CD4+ into T-regulatory 1 cells. T-regulatory 1 cells suppress the immune system.

The protein encoded by EIF4G1 a component of the multi-subunit protein complex EIF4F. This complex facilitates the recruitment of mRNA to the ribosome, which is a rate-limiting step during the initiation phase of protein synthesis. The recognition of the mRNA cap and the ATP-dependent unwinding of 5′-terminal secondary structure is catalyzed by factors in this complex. The subunit encoded by this gene is a large scaffolding protein that contains binding sites for other members of the EIF4F complex. A domain at its N-terminus can also interact with the poly(A)-binding protein, which may mediate the circularization of mRNA during translation. Pathogenic DNA variants within this gene dysregulate the recruitment of mRNA to ribosomes and is associated with pathophysiology of rheumatoid arthritis.

In some embodiments, the phenotype ranking is combined with biomimetic digital twin analysis. In some embodiments, genotype-phenotype ranked variants using specific key terms that described the phenotype of rheumatoid arthritis using a phenotype-driven ranking filter for each patient sample are downloaded. The data is then exported into the biomimetic digital twin ecosystem for analysis. 3172 VUSs in patient samples analyzed, but not found in controls, were identified, as shown in Appendix 2. Hidden or “dark data” for DNA variants in six genes classified as VUSs in patient samples were identified. The genes often found in patient samples included genes HIF1A, HLA-DOA, PTGER3, HIPK3, TGFBR3 and HIF1A-AS3 (Tables 1 to 2). The functional descriptions, category, and Functionality annotation score obtained from a database annotating and predicting human genes, are provided in Table 5. Functionality annotation score allows a quantitative assessment of a gene's annotation status by exploiting the gene's information in the database. The degree of accumulated knowledge for a given gene measured by Functionality annotation score was correlated with the number of publications for a gene, and with the seniority of this entry in the HUGO Gene Nomenclature Committee (HGNC) database.

TABLE 4
Biomimetic digital twin output identifying six genes closely
associated with the pathophysiology of rheumatoid arthritis

| Gene | | Protein | Translation | CADD | |
|---|---|---|---|---|---|
| Symbol | Transcript Variant | Variant | Impact | Score | Sample |
| HIF1A | c.2038C > G; c.2107C > G; | p.Q680E; | missense | 16.54 | 10461 |
| | c.2035C > G; n.213 + 9755G > C | p.Q703E; | | | |
| | | p.Q679E | | | |
| HIF1A | c.35 + 2003delT; c . −9delT | | | 3.912 | 10430 |
| HIF1A | c.1256C > T; c.1253C > T; | p.T419I; | missense | 20.3 | 10444 |
| | c. 1325C > T; n.213 + | p.T418I; | | | |
| | 12795G > A | p.T442I | | | |
| HIF1A | c. 1256C > T; c.1253C > T; | p.T419I; | missense | 20.3 | 10454 |
| | c.1325C > T; n.213 + 12795G > A | p.T418I; | | | |
| | | p.T442I | | | |
| HIF1A | c.151G > C; n.214-3477C > G; | p. V74L; | missense | 23.4 | 10431 |
| | c.220G > C; c.148G > C | p.V51L; | | | |
| | | p. V50L | | | |
| HIF1A | c.44delT; c.35 + 2055delT | p.L15* | frameshift | 20.2 | 10500 |
| HIF1A | c.44delT; c.35 + 2055delT | p.L15* | frameshift | 20.2 | 10500 |
| HIF1A-AS3 | c.2038C > G; c.2107C > G; | p.Q680E; | missense | 16.54 | 10461 |
| | c.2035C > G; n.213 + | p.Q703E; | | | |
| | 9755G > C | p.Q679E | | | |
| HIF1A-AS3 | c.1256C > T; c.1253C > T; | p.T419I; | missense | 20.3 | 10444 |
| | c.1325C > T; n.213 + | p.T418I; | | | |
| | 12795G > A | p.T442I | | | |
| HIF1A-AS3 | c.1256C > T; c.1253C > T; | p.T419I; | missense | 20.3 | 10454 |
| | c. 1325C > T; n.213 + | p.T418I; | | | |
| | 12795G > A | p.T442I | | | |
| HIF1A-AS3 | c. 151G > C; n.214-3477C > G; | p.V74L; | missense | 23.4 | 10431 |
| | c.220G > C; c.148G > C | p.V51L; | | | |
| | | p. V50L | | | |
| HIF1A-AS3 | c.2355G > A; n.213 + 3929C > T; | p.G784G; | synonymous | <10 | 10500 |
| | c.2424G > A; c.*17G > A; | p.G785G; | | | |
| | c.2352G > A | p.G808G | | | |
| HIPK3 | c.509G > A | p.G170E | missense | 20.7 | 10447 |
| HIPK3 | c.732A > G | p.I244M | missense | 23.3 | 10491 |
| HIPK3 | c.3511C > T; c.3448C > T | p.R1171C; | missense | 28.2 | 10436 |
| | | p.R1150C | | | |
| HIPK3 | c. 1499G > A | p.S500N | missense | 20.7 | 10500 |
| HLA-DOA | c.313C > T | p.R105C | missense | 23.2 | 10447 |
| HLA-DOA | c.313C > T | p.R105C | missense | 23.2 | 10455 |
| HLA-DOA | c.108C > T | p.P36P | synonymous | <10 | 10465 |
| HLA-DOA | c.3G > A | p.M1I | start loss | 24.7 | 10466 |
| PTGER3 | n.1316 + 59326delC; c.1185delC; | p.N395fs*9 | frameshift | 19.64 | 10491 |
| | c.*104delC; c.1104 + 784delC; | | | | |
| | n.1343 + 784delC; | | | | |
| | c.1077 + 59326delC; | | | | |
| | c.*23 + 784delC | | | | |
| PTGER3 | c.1105T > C; c.1077 + 59246T > C; | p.L369L | synonymous | <10 | 10446 |
| | c.*24T > C; n.1316 + 59246T > C; | | | | |
| | c.1104 + 704T > C; c.*23 + 704T > C; | | | | |
| | n.1343 + 704T > C | | | | |
| PTGER3 | n.1317-20553C > T; c.1124C > T; | p.P375L | missense | 20.3 | 10446 |
| | c.1078 − 20553C > T; | | | | |
| | n.1316 + 37963C > T; | | | | |
| | c.1077 + 37963C > T | | | | |
| PTGER3 | n.1316 + 59326delC; c.1185delC; | p.N395fs*9 | frameshift | 19.64 | 10440 |
| | c.*104delC; c.1104 + 784delC; | | | | |
| | n.1343 + 784delC; | | | | |
| | c.1077 + 59326delC; c.*23 + 784delC | | | | |
| TGFBR3 | c.2329C > T; c.2326C > T; | p.P777S; | missense | 22.8 | 10447 |
| | n.2813C > T | p.P776S | | | |
| TGFBR3 | c.2365A > T; n.2852A > T; | p.I790F; | missense | 29.8 | 10451 |
| | c.2368A > T | p.I789F | | | |
| TGFBR3 | c.886G > T; n.1370G > T | p.A296S | missense | 22.9 | 10446 |
| TGFBR3 | n.442A > G; c.55A > G | p.T19A | missense | <10 | 10454 |
| TGFBR3 | c.464A > G; n.948A > G | p.H155R | missense | 19.22 | 10484 |
| TGFBR3L | c.917C > A | p.S306* | stop gain | 39 | 10451 |

TABLE 5
Six genes that are classified as VUSs and associated
with the pathophysiology of rheumatoid arthritis.

| | | | Functionality |
|---|---|---|---|
| | | | annotation |
| Symbol | Description | Category | score |
| HIF1A | Hypoxia Inducible Factor 1 Subunit Alpha | Protein Coding | 57 |
| HIF1A-AS3 | HIF1A Antisense RNA 3 | RNA Gene | 18 |
| HIPK3 | Homeodomain Interacting Protein Kinase 3 | Protein Coding | 45 |
| HLA-DOA | Major Histocompatibility Complex, Class II, | Protein Coding | 45 |
| | DO Alpha | | |
| PTGER3 | Prostaglandin E Receptor 3 | Protein Coding | 53 |
| TGFBR3 | Transforming Growth Factor Beta Receptor 3 | Protein Coding | 54 |

In some embodiments, an HIF1A gene encodes the alpha subunit of transcription factor hypoxia-inducible factor-1 (HIF-1), which is a heterodimer composed of an alpha and a beta subunit. HIF-1 functions as a master regulator of cellular and systemic homeostatic response to hypoxia by activating transcription of many genes, including those involved in energy metabolism, angiogenesis, apoptosis, and other genes whose protein products increase oxygen delivery or facilitate metabolic adaptation to hypoxia.

In some embodiments, 18 VUSs, and 12 different proteins within the HIF1A gene in patients analyzed were identified, but one was classified as a missense mutation.

In some embodiments, an HLA-DOA gene is a protein-coding gene that belongs to the HLA class II alpha chain paralogues. It is a non-classical HLA gene that forms a heterodimer with HLA-DOB. The heterodimer, HLA-DOA, is found in lysosomes in B cells and regulates HLA-DM-mediated peptide loading on MHC class II molecules. One study identified an independent risk of a synonymous mutation at HLA-DOA on anti-citrullinated protein autoantibody (ACPA)-positive rheumatoid arthritis risk.

In some embodiments, 3 VUSs, and 3 different proteins within the HLA-DOA gene in patients analyzed were identified. One is a missense mutation, one is a synonymous variant, and one is a start-loss mutation.

In some embodiments, a PTGER3 gene is a receptor for prostaglandin E2 (PGE2). The activity of this receptor can couple to both the inhibition of adenylate cyclase mediated by G (i) proteins, and to an elevation of intracellular calcium. Prostanoid receptors are activated by the endogenous ligands prostaglandin (PG) D2, PGE2, PGF2alpha, PGH2, prostacyclin (PGI2) and thromboxane A2. Cyclooxygenase (COX) converts arachidonic acid to PGH2, from which other prostaglandins are synthesized. PGE2 is induced with IL-1, which also enhances the production of parathyroid hormone-related protein (PTHrP). The induction of PGE2 by IL-1alpha appears to be an important component of the PTHrP production of the inflammatory process in synovial tissues from patients with RA.

In some embodiments, within the PTGER3 gene, 21 VUSs that were transcribed into six different proteins were identified. All of these proteins are predicted to be missense mutations. A missense mutation is a DNA change that replaces an amino acid in a protein with a different one. Missense mutations are also known as nonsynonymous mutations. Some missense mutations have little to no effect on the protein's function, while others can alter it. For example, a missense mutation in the caveolin-3 gene is associated with limb-girdle muscular dystrophy in humans. Another missense mutation in the PAX3 gene can cause Klein-Waardenburg syndrome, which includes limb abnormalities. A different missense mutation in the same amino acid residue can cause craniofacial-deafness-hand syndrome, a more severe disorder.

In some embodiments, HIPK3 enables protein serine/threonine kinase activity, is involved in mRNA transcription, it provides negative regulation of apoptosis and aids in protein phosphorylation. DNA variants within this gene appear to play a role in the development of rheumatoid arthritis.

In some embodiments, 5 VUSs that encoded 5 different proteins within the HIPK3 gene in patients analyzed were identified. Proteins were classified as missense variants.

In some embodiments, a TGFBR3 locus encodes the transforming growth factor (TGF)-beta type III receptor. The encoded receptor is a membrane proteoglycan that often functions as a co-receptor with other TGF-beta receptor superfamily members. Ectodomain shedding produces soluble TGFBR3, which may inhibit TGFB signaling. Variants with this gene likely play an indirect role in the pathophysiology of rheumatoid arthritis. 12 VUSs, encoding 7 proteins within the TGFBR3 gene in patients analyzed were identified. Variants were classified as missense mutations.

In some embodiments, HIF1A-AS3 is an RNA gene and is affiliated with the lncRNA class of molecules. It appears to play an indirect role in the development of rheumatoid arthritis. 12 VUSs, encoding 11 proteins within the HIF1A-AS3 gene in patients analyzed are identified, one of these variants is classified as missense mutation. Another mutation is classified as a synonymous variant.

In this example, additional evidence was provided to support the use of incorporating whole exome sequencing, phenotype-driven ranking filters with knowledge engineering via the use of biomimetic digital twins, a database on annotated human genes, and a phenotype-dependent variants prioritizer, to provide a greater understanding of the molecular mechanism of disease. Furthermore, these results are beginning to show the value of multiomics and the use of biomimetic digital twins for the potential reclassification of VUSs.

Five pathogenic, and one likely pathogenic, DNA variants in 8/25 patient samples analyzed, but not in 25 control samples, were identified.

Clinical molecular laboratory directors face immense challenges in making decisions on reporting out VUSs. The number of VUSs identified in exome sequencing can vary significantly from person to person. Whole exome sequencing typically identifies thousands of genetic variants within exons.

Many of these variants may be common in the population and are well-studied, while others may be rare or previously unreported. VUSs are those genetic variants whose significance in relation to disease or health outcomes is not understood. These require further investigation, functional studies, or larger population studies to determine their clinical relevance.

The number of VUSs identified in whole exome sequencing depends on various factors, including the individual's genetic background, ethnicity, family history, and the specific criteria used to classify variants as VUSs. Additionally, the depth and accuracy of sequencing, as well as the bioinformatics tools and databases used for variant interpretation, can also influence the number of VUSs identified.

In clinical settings, genetic counselors, geneticists, oncologists, and other healthcare specialties carefully assess and interpret variants identified through whole exome sequencing to provide patients with the most accurate information regarding their potential health implications. As the understanding of the human genome and the functional significance of genetic variants continues to evolve, the interpretation of VUSs will also change over time.

Genotype-phenotype ranking and a biomimetic digital twin engine were used to identify 3172 VUSs potentially associated with the pathophysiology of rheumatoid arthritis.

In addition to the biomimetic digital twin engine, a database on annotated human genes and a phenotype-dependent variants prioritizer were incorporated into the analysis. Four VUSs, in genes HIF1A, HLA-DOA, PTGER3, HIPK3, are identified, which are directly related to the development of rheumatoid arthritis (Table 6).

TABLE 6
Four example genes, classified as VUSs, directly associated with rheumatoid arthritis

| | | | | | | Global | | | |
|---|---|---|---|---|---|---|---|---|---|
| | | | | | | Rank | | | Average |
| | | | Functionality | | Matched | (Total | | | Disease |
| | | | Annotation | Matched | Phenotypes | Genes | | | Causing |
| Symbol | Description | Category | Score | Phenotypes | Scount | 4892) | −LOG10(P) | Score | Likelihood |
| HLA- | Major Histocompatibility | Protein | 45 | “rheumatoid | 1 | 199 | 2.21 | 4.79 | 35.57% |
| DOA | Complex, Class II, DO | Coding | | arthritis” | | | | | |
| | Alpha | | | | | | | | |
| HIF1A | Hypoxia Inducible Factor | Protein | 57 | “rheumatoid | 1 | 252 | 2.11 | 4.21 | 61.97% |
| | 1 Subunit Alpha | Coding | | arthritis” | | | | | |
| PTGER3 | Prostaglandin E Receptor | Protein | 53 | “rheumatoid | 1 | 3453 | 0.97 | 0.93 | 58.11% |
| | 3 | Coding | | arthritis” | | | | | |
| HIPK3 | Homeodomain Interacting | Protein | 45 | “rheumatoid | 1 | 3622 | 0.95 | 0.86 | 64.66% |
| | Protein Kinase 3 | Coding | | arthritis” | | | | | |

The HIF1A gene is found in 7/7 patient samples, HLA-DOA is present in 4 samples, the PTGER3 VUS was identified in 4 patient samples, whereas 4/25 patients had a HIPK3 VUS.

All but one of the VUSs identified within the HIF1A gene were classified as a missense mutation. Missense variants are a genetic alteration in which a single base pair substitution alters the genetic code in a way that produces an amino acid that is different from the usual amino acid at that position. Many missense variants will alter the function of the protein and be disease causing.

Hyperplasia of synovial fibroblasts, infiltration with inflammatory cytokines, and tissue hypoxia are major characteristics of rheumatoid arthritis. Interleukin 33 (IL-33) is an inflammatory cytokine exacerbating the disease severity of rheumatoid arthritis. Hypoxia-inducible factor-1α (HIF-1A) shows increased expression in RA synovium and could regulate several inflammatory cytokine productions. Elevated levels of IL-33 are shown in RA patient synovial fluids. HIF-1A appears to promote the activation of the signaling pathways controlling IL-33 production, particularly the p38 and ERK pathways. IL-33 in turn could induce more HIF-1α expression, thus forming a HIF-1α/IL-33 regulatory circuit that would perpetuate the inflammatory process in rheumatoid arthritis.

In some embodiments, three VUSs that encoded three different proteins within the HLA-DOA gene were identified.

One is a missense mutation, one is a synonymous variant, and one is a start-loss mutation. Start-loss mutations are a point mutation in the ATG start codon of a transcript that reduces or eliminates protein production. The elimination or reduction of a functional protein is most likely a disease-causing DNA variant. A synonymous mutation is a genetic change that alters a gene's DNA sequence but not the protein sequence it encodes. Synonymous mutations have traditionally been considered neutral mutations because they don't change the amino acid that is translated. However, recent studies suggest that synonymous mutations can have a significant impact on RNA stability, RNA folding, translation, and co-translational protein folding.

In prior art, Okada, Y, et al, conducted a large-scale MHC fine-mapping analysis of rheumatoid arthritis patients in a Japanese population (6,244 RA cases and 23,731 controls) population by using HLA imputation, followed by a multi-ethnic validation study including east Asian and European populations (n=7,097 and 23,149, respectively). They identified an independent risk of a synonymous mutation at HLA-DOA, a non-classical HLA gene, on anti-citrullinated protein autoantibody (ACPA)-positive RA risk (p=1.4×10(−9)), which demonstrated a cis-expression quantitative trait loci (cis-eQTL) effect on HLA-DOA expression. Trans-ethnic comparison revealed different linkage disequilibrium (LD) patterns in HLA-DOA and HLA-DRB1, explaining the observed HLA-DOA variant risk heterogeneity among ethnicities, which is most evident in the Japanese population. Although previous HLA fine-mapping studies have identified amino acid polymorphisms of the classical HLA genes as driving genetic susceptibility to disease, this study additionally identified the dosage contribution of a non-classical HLA gene, HLA-DOA to RA disease etiology.

Within the PTGER3 gene, four different proteins were identified. These were missense variants.

The protein encoded by the PTGER3 gene is a member of the G-protein coupled receptor family. This protein is one of four receptors identified for prostaglandin E2 (PGE2). This receptor may have many biological functions, which involve digestion, nervous system, kidney reabsorption, and uterine contraction activities. In inflamed joints of rheumatoid arthritis, PGE2) is highly expressed, and IL-10 and IL-6 are also abundant. PGE 2) is a well-known activator of the cAMP signaling pathway, and there is functional crosstalk between cAMP signaling and the Jak-STAT signaling pathway.

Within the HIPK3 gene, five VUSs that produced five proteins are identified, the encoded proteins are classified as missense mutations.

HIPK3 encodes a homeodomain interacting protein kinase 3. This enables protein serine/threonine kinase activity. It is involved in mRNA transcription, cell proliferation, inflammation, negative regulation of the apoptotic process, and protein phosphorylation.

Over-expression of HIPK3 protein in immune cells in rheumatoid arthritis patients has also been reported.

Two of these VUSs, TGFBR3 and HIF1A-AS3 are indirectly related to the pathophysiology of rheumatoid arthritis (Table 7).

TABLE 7
Two genes, classified as VUSs, indirectly related
to the pathophysiology of rheumatoid arthritis
Results

| | | | | Functionality | |
|---|---|---|---|---|---|
| Implicated | Implicating | | | Annotation | Matched |
| Symbol | Symbol | Description | Category | Score | Phenotypes |
| TGFBR3 | TNF | Tumor Necrosis Factor | Protein | 61 | “rheumatoid |
| | | | Coding | | arthritis” |
| TGFBR3 | IL10 | Interleukin 10 | Protein | 56 | “rheumatoid |
| | | | Coding | | arthritis” |
| TGFBR3 | IL6 | Interleukin 6 | Protein | 60 | “rheumatoid |
| | | | Coding | | arthritis” |
| TGFBR3 | STAT4 | Signal Transducer And | Protein | 55 | “rheumatoid |
| | | Activator Of | Coding | | arthritis” |
| | | Transcription 4 | | | |
| TGFBR3 | TGFB1 | Transforming Growth | Protein | 61 | “rheumatoid |
| | | Factor Beta 1 | Coding | | arthritis” |
| HIF1A-AS3 | HIF1A | Hypoxia Inducible Factor | Protein | 57 | “rheumatoid |
| | | 1 Subunit Alpha | Coding | | arthritis” |
| HIF1A-AS3 | SNAPC1 | Small Nuclear RNA | Protein | 39 | “rheumatoid |
| | | Activating Complex | Coding | | arthritis” |
| HIF1A-AS3 | HIF1A-AS2 | HIF1A Antisense RNA 2 | RNA Gene | 23 | “rheumatoid |
| | | | | | arthritis” |

| | | Implicated Gene | | Implicating Gene | |
|---|---|---|---|---|---|
| Results | | | Average | | Average |
| Matched | Global Rank | | Disease | | Disease |
| Phenotypes | (Total Genes | Score | Causing | Score | Causing |
| Count | 4892) | (Implicated) | Likelihood | (Implicating) | Likelihood1 |
| 1 | 26 | 1.35 | 25.53% | 0.77 | 70.41% |
| 1 | 5 | 1.35 | 25.53% | 0.68 | 63.14% |
| 1 | 9 | 1.35 | 25.53% | 0.64 | 56.07% |
| 1 | 29 | 1.35 | 25.53% | 0.46 | 73.39% |
| 1 | 130 | 1.35 | 25.53% | 0.46 | 42.49% |
| 1 | 252 | 0.10 | 0.00% | 0.09 | 61.97% |
| 1 | 1354 | 0.10 | 0.00% | 0.03 | 51.67% |
| 1 | 4369 | 0.10 | 0.00% | 0 | 0.00% |

The TGFBR3 DNA variant was found in 5/25 patient samples, and the HIF1A-AS3 VUS was also present in 5/25 samples.

Twelve VUSs within the TGFBR3 gene that encoded seven different proteins were identified. These proteins are missense mutations.

This TGFBR3 locus encodes the transforming growth factor (TGF)-beta type III receptor. The encoded receptor is a membrane proteoglycan that often functions as a co-receptor with other TGF-beta receptor superfamily members. Ectodomain shedding produces soluble TGFBR3, which may inhibit TGFB signaling. Decreased expression of this receptor has been observed in various cancers. Alternatively spliced transcript variants encoding different isoforms are identified for this gene. Diseases associated with TGFBR3 include Familial Cerebral Saccular Aneurysm and Priapism. Among its related pathways are Apoptotic Pathways in Synovial Fibroblasts and Negative regulation of FGFR3 signaling.

TGFBR3 is indirectly related to the development of RA by interacting with pathways including TNF. This gene encodes a multifunctional proinflammatory cytokine that belongs to the tumor necrosis factor (TNF) superfamily.

TGFBR3 also interacts with pathways for IL-6. This gene encodes a cytokine that functions in inflammation and the maturation of B cells. The protein is primarily produced at sites of acute and chronic inflammation, where it is secreted into the serum and induces a transcriptional inflammatory response through interleukin 6 receptor, alpha.

TGFBR3 plays an indirect role in the development of rheumatoid arthritis by interacting with the TGHB1 gene pathway. This gene encodes a secreted ligand of the TGF-beta (transforming growth factor-beta) superfamily of proteins. Ligands of this family bind various TGF-beta receptors leading to recruitment and activation of SMAD family transcription factors that regulate gene expression. The encoded preproprotein is proteolytically processed to generate a latency-associated peptide (LAP) and a mature peptide and is found in either a latent form composed of a mature peptide homodimer, a LAP homodimer, and a latent TGF-beta binding protein, or in an active form consisting solely of the mature peptide homodimer. The mature peptide may also form heterodimers with other TGFB family members. This encoded protein regulates cell proliferation, differentiation, and growth, and can modulate expression and activation of other growth factors.

Gene pathways including IL-10, play a role in the pathophysiology of RA. TGHBR3 plays an indirect role in the activation of this pathway. IL-10 encodes a cytokine that is produced primarily by monocytes and to a lesser extent by lymphocytes. This cytokine has pleiotropic effects in immunoregulation and inflammation. It down-regulates the expression of Th1 cytokines, MHC class II antigens, and costimulatory molecules on macrophages. It also enhances B cell survival, proliferation, and antibody production. This cytokine can block NF-kappa B activity and is involved in the regulation of the JAK-STAT signaling pathway.

TGFBR3 also plays an indirect role in the development of RA by interacting with the Stat-4 pathway. This protein encoded by this gene is a member of the STAT family of transcription factors. In response to cytokines and growth factors, STAT family members are phosphorylated by the receptor associated kinases, and then form homo- or heterodimers that translocate to the cell nucleus where they act as transcription activators. This protein is essential for mediating responses to IL12 in lymphocytes and regulating the differentiation of T helper cells. DNA variants in this gene may be associated with systemic lupus erythematosus and rheumatoid arthritis.

Of the eleven encoded proteins from the HIFA-AS3 VUSs, all but one are potentially disease causing and associated with the pathophysiology of rheumatoid arthritis.

The HIF1A-AS3 gene is also indirectly related to the pathophysiology of RA by interacting with pathways that include the genes HIF1A, SNAPC1, and HIF1A-AS2.

The HIF1A gene encodes the alpha subunit of transcription factor hypoxia-inducible factor-1 (HIF-1), which is a heterodimer composed of an alpha and a beta subunit. HIF-1 functions as a master regulator of cellular and systemic homeostatic response to hypoxia by activating transcription of many genes, including those involved in energy metabolism, angiogenesis, apoptosis, and other genes whose protein products increase oxygen delivery or facilitate metabolic adaptation to hypoxia. Hypoxia Inducible Factors (HIFs) are transcription factors that are activated in response to decreased oxygen availability in the cellular environment. Tissue hypoxia are major characteristics of rheumatoid arthritis.

HIF1A-AS3 plays an indirect role in the gene pathway of SNAPC1. The SNAPC1 gene product is a small nuclear RNA activating complex polypeptide 1. It is predicted to enable sequence-specific DNA binding activity. It is also predicted to be involved in snRNA transcription by RNA polymerase II and snRNA transcription by RNA polymerase III. The SNAPC1 pathway plays a role in the development of rheumatoid arthritis.

HIF1A-AS2 (HIF1A Antisense RNA 2) is an RNA gene and is affiliated with the lncRNA class of molecules. HIF1A-AS3 potentially plays an indirect role in the pathophysiology of RA by interacting with the HIF1A-AS2 pathway.

The identification of these VUSs doesn't confirm that they play a role in the development of rheumatoid arthritis, but the fact that most of their encoded proteins are classified as non-functional strongly suggests that they are highly likely to play some role in the pathophysiology of this disorder.

Proving that a VUS is pathogenic or likely pathogenic involves a process of variant interpretation and assessment. This process typically involves multiple steps and considerations, including:
1. Clinical—The first step is to gather clinical information about the individual who underwent genetic testing. This includes their medical history, family history, presenting symptoms, and any relevant clinical findings. Understanding the phenotype associated with the variant can provide valuable context for its interpretation.
2. Classification Guidelines—Variants identified through genetic testing are classified according to established guidelines, such as those provided by the American College of Medical Genetics and Genomics (ACMG) or the Association for Molecular Pathology (AMP). Variants are categorized into five main classes: pathogenic, likely pathogenic, variants of unknown clinical significance, likely benign, and benign.
3. Functional studies—These may be conducted to assess the impact of the variant on protein function or expression. These studies can provide direct evidence of the variant's pathogenicity by demonstrating its effect on cellular processes or protein function. In silico applications can also be used to determine whether a protein is functional or non-functional proteins.
4. Population Frequency—Variants that are rare in the general population are more likely to be pathogenic, especially if they are found in genes known to be associated with disease. Population databases such as the Exome Aggregation Consortium (ExAC) or the Genome Aggregation Database (gnomAD) can be used to assess the frequency of the variant in different populations.
5. Segregation—In families with multiple affected individuals, segregation analysis can be used to determine whether the variant co-segregates with the disease phenotype. If the variant is found in affected family members but not in unaffected individuals, this provides strong evidence of its pathogenicity.
6. In Silico Predictions—Computational algorithms and bioinformatics tools can be used to predict the functional impact of a variant based on its location within the gene and its effect on protein structure. While these tools are not definitive proof of pathogenicity, they can provide supporting evidence.

It's important to note that proving pathogenicity or likely pathogenicity for a VUS is often challenging and may require multiple lines of evidence. In many cases, variants initially classified as VUSs may be reclassified over time as additional evidence becomes available. Therefore, ongoing research and updates to variant databases are essential for improving the understanding of genetic variation and its clinical significance.

The reclassification of VUSs is one of the most significant challenges in genetics and genomics today.

A Biomimetic Digital Twin analysis is very powerful in its ability to analyze small, wide data sets and identify dark data. Additional studies are required to discover the role of ethnicity, if any, in the pathogenesis of rheumatoid arthritis.

In conclusion, these results suggest that by using multiomics and digital twins, more insight into the development of RA can be gained. Potentially the process of reclassifying VUSs can be started. The reclassification of VUSs will play a critical role in complex molecular diagnostics and drug development.

### Example 3: Applications to Inherited Breast Cancer and the Potential Reclassification of Variants of Unknown Clinical Significance

Biomimetic digital twins have been incorporated with the multiomics platform to potentially clarify the pathogenesis of inherited breast cancer. It was accomplished by identifying dark data—complex relationships that are not visible in bioinformatics platforms—as either directly or indirectly related to the development of inherited breast cancer.

These results suggested that by using biomimetic digital twins and a Multiomics platform, the process of reclassifying variants of unknown clinical significance (VUS) can be started.

Published papers identified over 13,000 gene inter-relationships potentially associated with this disorder.

In this study, variants of unknown clinical significance (VUSs) in patient samples, absent from controls, were identified. 133 genes, classified as VUSs, potentially associated with the development of inherited breast cancer have been identified. 27 genes, classified as VUSs, potentially associated with inherited breast cancer, have been identified through pathway analysis. 7 genes, classified as VUSs, have been identified with a poor prognosis. Six lincRNAs potentially associated with the development of inherited breast cancer have been identified.

These results suggest that by using digital twins and multiomics, more insight into the development of inherited breast cancer can be gained.

### Example 4: Applications to Episodic Eosinophilia and Angioedema (EAE) and the Potential Reclassification of Variants of Unknown Clinical Significance

The aim of this study is to use a novel approach to more clearly understand the pathophysiology of episodic eosinophilia and angioedema (EAE), a rare form of hypereosinophilic syndrome characterized by multilineage cycling associated with a variety of clinical symptoms. Ultimately, this information can be used to identify new therapeutic targets. A biomimetic digital twin analysis approach will be used to analyze data from a cohort of patients with EAE enrolled on an NIH IRB-approved protocol to study eosinophilia (94-I-0079). This approach offers significant advantages over traditional approaches to data analysis both because it employs a specialized form of artificial intelligence to identify “dark data” (i.e., data that is disconnected, unseen, unexplored and as a result, unanalyzed) and because, in contrast to traditional AI, large numbers of subjects are not required. Clinical, multiomic and biomarker data from patients with EAE will be provided by NIAID investigators. This data will be analyzed using a process that includes biomimetic digital twins, phenotype ranking, general knowledge graphs, expert knowledge graphs, a searchable, integrative database that provides comprehensive, information on all annotated and predicted human genes, and a comprehensive phenotype-dependent DNA variant/gene prioritizer, to identify causal DNA variants with phenotypes (genotype/phenotype). This provides search and scoring capabilities, proficiently matching DNA variant-containing genes to submitted disease/symptom/phenotype keywords. This output will infer direct as well as indirect links between genes and phenotypes.

These results suggest that by using digital twins and multiomics, more insight into the pathophysiology of episodic eosinophilia and angioedema (EAE), or a cycling disorder can be gained.

Example 5: The Application of Multiomics, Phenotype-Driven Variant Analysis, Biomimetic Digital Twins, and Genotype-Phenotype Relationships to Understand the Molecular Mechanisms of Disease More Clearly, and to Enable Genetic Variants of Unknown Clinical Significance (VUSs) to be Reclassified as Pathogenic, Likely Pathogenic, Benign, or Likely Benign and to Reduce the Time and Cost to Drug Development

In accordance with at least some embodiments disclosed herein is the realization that across multiple domains of science, engineering, and medicine, excitement is growing about the potential of digital twins to transform scientific research, industrial practices, and many aspects of daily life. A digital twin combines computational models with a physical counterpart to create a system that is dynamically updated through bidirectional data flows as conditions change. Going beyond traditional simulation and modeling, digital twins could enable improved precision medicine and healthcare by more clearly understanding the pathophysiology of disease. In accordance with at least some embodiments disclosed herein is the realization that the foundational research and resources needed to support the development of digital twin technologies is ongoing.

Biomimetic digital twins apply many of the recommendations from the NAS report, including the conclusion that new theories and methods are required to address the multi-dimensional, multi-scale characteristics of problems in modeling and advanced analytics in general, and in biomedicine in particular.

In some embodiments, a biomimetic digital twin ecosystem is incorporated into advanced multiomics experimental protocols. The biomimetic knowledge engineering methodology are utilized for creating an ecosystem of digital twins that implement real-world reasoning principles and that analyzed data that is raw and in its original state-meaning that no cleansing or normalization that removed outliers and hide relationships and impacts within data sets are performed. The use of this methodology has both leveraged and utilized dark data and has enabled unexpected discovery.

In one study, the research using whole exome sequencing, phenotype-driven ranking analysis, a biomimetic digital twin analysis, a database on annotated human genes, and a phenotype-dependent variants prioritizer to identify dark data associated with the molecular profile of the complex multifactorial disorder are performed. Significantly, additional results demonstrating that biomimetic digital twins and multiomics can play a role in the clarification in the pathophysiology of a complex genetic disorder are provided. Furthermore, it demonstrated the potential role of using the platform in identifying variants of unknown clinical significance (VUSs) potentially associated with the development of rheumatoid arthritis and suggested a way to potentially reclassify VUSs as pathogenic, likely pathogenic, benign, or likely benign.

The present invention demonstrated, for the first time, the ability to combine multiomics, phenotype driven ranking analysis, biomimetic digital twins, and genotype-phenotype relationships, to understand more clearly the pathophysiology of disease(s). Upon completion of identifying VUSs closely associated with the phenotype of the disease(s) studied, by using a systematic and evidence-based approach, drawing upon multiple lines of evidence and expertise to make informed decisions about the variant's clinical significance, VUSs can be reclassified. In this step, clinical information, data collection, literature review, classification guidelines, functional studies, population frequency, segregation studies, in silico predictions, documentation and reporting, and periodic reassessment can be involved. Furthermore, the platform output can be used to identify new therapeutic targets, and to reduce the time and cost of drug development.

The present invention allows one to understand the pathophysiology of any disease more clearly, to correlate DNA or RNA variants with patient symptoms, to reclassify variants of unknown clinical significance, and to reduce the time and cost of drug development.

Process from the selection of the disease to be studied, to exome/whole genome sequencing, phenotype ranking, biomimetic digital twin ecosystem, genotype-phenotype rankings, the reclassification of VUSs, and to reducing the time and cost to drug development.
1) Identify the disease to be studied.
2) Extract nucleic acids, protein, and other types of molecules from patient samples.
3) Perform a library prep for whole genome or clinical exome sequencing.
4) Export raw data into a secondary analysis pipeline.
5) Export genomic files into a tertiary analysis platform.
6) Download DNA variants.
7) Download phenotype ranked DNA variants.
8) Export DNA variant and phenotype ranked files into the biomimetic digital twin ecosystem.
9) Export general knowledge graphs into an ecosystem data lake. These graphs are what is known about the disease in relevant databases (ie PubMed).
10) Export expert knowledge graphs into the biomimetic digital twin ecosystem. Expert knowledge graphs are designed by experts in the disease studied.
11) Export into the biomimetic digital twin ecosystem data lake a searchable, integrative databases that provides information on annotated and predicted human genes. The knowledge base will automatically integrate gene-centric data from ˜300 web sources, including genomic, transcriptomic, proteomic, genetic, clinical, and functional information.
12) Export into the biomimetic digital twin ecosystem data lake, a phenotype-dependent DNA variant/gene prioritizer, to identify causal DNA variants with phenotypes (genotype-phenotype). This provides search and scoring capabilities, proficiently matching DNA variant-containing genes to submitted disease/symptom/phenotype keywords. This output will infer direct as well as indirect links between genes and phenotypes.
13) Run multidimensional data residing in the ecosystem data lake, using knowledge engineering for biomimetic digital twin analysis. In the real-world lab of delivering solutions, we have evolved the methods of real-world complexity modeling, real-world reasoning, real-world learning, and other adaptation methods, to explore highly complex, multidimensional, and multiscale problem domains for global organizations. This is called a biomimetic digital twin ecosystem. The use of this methodology has both leveraged and utilized dark data and has enabled unexpected discoveries. This is used in domains that are complex, multidimensional, multiscale, or dynamic in more clearly understand the pathophysiology of disease(s).
14) The output will identify dark or hidden data associated with genotype-phenotype rankings. The output will also rank VUSs most closely associated with the phenotype of the disease being studied. This will be used to begin to reclassify these variants as pathogenic, likely pathogenic, benign, or likely benign.
15) Reclassifying VUSs requires a systematic and evidence-based approach, drawing upon multiple lines of evidence and expertise to make informed decisions about the variant's clinical significance.
   Clinical—The first step is to gather clinical information about the individual who underwent genetic testing. This includes their medical history, family history, presenting symptoms, and any relevant clinical findings. Understanding the phenotype associated with the variant can provide valuable context for its interpretation.
   Data Collection—Gather relevant data on the variant, including its genomic location, allele frequency in the population, conservation across species, predicted impact on protein structure and function, and any available clinical or functional evidence.
   Literature Review—Conduct a literature review to identify any published studies or databases reporting on the variant. This includes studies describing the variant's association with disease, functional assays assessing its impact, and population frequency data.
   Classification Guidelines—Variants identified through genetic testing are classified according to established guidelines, such as those provided by the American College of Medical Genetics and Genomics (ACMG) or the Association for Molecular Pathology (AMP). Variants are categorized into five main classes: pathogenic, likely pathogenic, variants of unknown clinical significance, likely benign, and benign.
   Functional studies—Perform functional assays to experimentally assess the variant's impact on gene function, protein structure, or molecular pathways. This may involve in vitro or in vivo experiments, such as cell-based assays, biochemical assays, or animal models.
   Population Frequency—Variants that are rare in the general population are more likely to be pathogenic, especially if they are found in genes known to be associated with disease. Population databases such as the Exome Aggregation Consortium (ExAC) or the Genome Aggregation Database (gnomAD) can be used to assess the frequency of the variant in different populations.
   In some implementations, in families with multiple affected individuals, segregation analysis can be used to determine whether the variant co-segregates with the disease phenotype. If the variant is found in affected family members but not in unaffected individuals, this provides strong evidence of its pathogenicity.
   in Silico Predictions—Computational algorithms and bioinformatics tools can be used to predict the functional impact of a variant based on its location within the gene and its effect on protein structure. While these tools are not definitive proof of pathogenicity, they can provide supporting evidence.
   Documentation and Reporting—Document the evidence supporting the reclassification of the variant and provide clear rationale for the updated classification. This information should be communicated effectively to relevant stakeholders, including healthcare providers, patients, and researchers.
   Periodic Reassessment—As new evidence emerges or additional data becomes available, periodically reassess variants to incorporate updated information and refine their classification status.
16) Once variants of unknown clinical significance (VUSs) are reclassified to either pathogenic or likely pathogenic, one can use these in new drug discovery. This will shorten the time and reduce the cost in new therapeutics coming to market for enhanced patient care and precision medicine.

This example disclosed application of multiomics, phenotype-driven variant analysis, biomimetic digital twins, and genotype-phenotype relationships to understand the molecular mechanisms of disease more clearly, and to enable genetic variants of unknown clinical significance (VUSs) to be reclassified as pathogenic, likely pathogenic, benign, or likely benign and to reduce the time and cost to drug development.

The description of the present application has been presented for purposes of illustration and description and is not intended to be exhaustive or limited to the invention in the form disclosed. Many modifications, variations, and alternative embodiments will be apparent to those of ordinary skill in the art having the benefit of the teachings presented in the foregoing descriptions and the associated drawings. The embodiments are chosen and described in order to explain the principles of the invention, the practical applications, and to enable others skilled in the art to understand the invention for various embodiments and to utilize the underlying principles and various embodiments with various modifications as are suited to the particular use contemplated. Therefore, it is to be understood that the scope of claims is not to be limited to the specific examples of the embodiments disclosed and that modifications and other embodiments are intended to be included within the scope of the appended claims.

### Illustration of the Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A computer-implemented method for genetic testing, comprising: obtaining information of a plurality of subject genetic variants that are identified from a plurality of biological samples of a plurality of patients who are diagnosed with a target disease; selecting a subset of subject genetic variants from the plurality of subject genetic variants based on a plurality of subject phenotypes of the target disease; ranking the subset of subject genetic variants based on the plurality of subject phenotypes to generate subject genetic variant information; obtaining subject medical information of the plurality of patients, including doctor-inputted description of the target disease collected from the plurality of patients; obtaining general genetic variant information, independently of the plurality of patients; applying a biomimetic information model to process the subject genetic variant information, the subject medical information, and the general genetic variant information of the target disease; and identifying a set of target genetic variants from the subset of subject genetic variants, the set of target genetic variants satisfying a variant selection criterion.

Clause 2. The method of clause 1, further comprising: assessing the set of target variants as therapeutic targets; and determining one or more compounds of a drug configured to treat the target disease.

Clause 3. The method of clause 2, wherein the set of target variants includes a first target variant, and assessing the set of target variants further comprising one or more of: gathering clinical information about the plurality of patients; classifying the first target variant to one of a set of classes including pathogenic, likely pathogenic, uncertain significance, likely benign, and benign; conducting functional study to assess an impact of the first target variant on a protein function or expression; assessing a frequency of the first target variant in one or more populations; determining whether the first target variant co-segregates with a predefined phenotype; predicting a functional impact of the first target variant based on a variant location within a corresponding gene and an effect on a protein structure; and identifying a pathway affected by the first target variant and contributing to one or more phenotypes of the target disease.

Clause 4. The method of any of clause 2 or 3, wherein the one or more compounds of the drug include small molecules, antibodies, gene therapies, an immunotherapy, a chemotherapy, a radiation therapy, a hormone therapy, a photodynamic therapy, a targeted therapy, polynucleotide, natural compound, immune modulator, bone marrow therapy, stem cell therapy, surgery therapy, induction therapy, maintenance therapy, or a combination thereof.

Clause 5. The method of any of clauses 1-4, wherein the general genetic variant information includes a first knowledge graph that couples the target disease and a plurality of first biomedical terms semantically to one another based on a knowledge database.

Clause 6. The method of Clause 5, wherein the public knowledge database includes a collection of official peer-reviewed publications, the method further comprising: semantically analyzing a subset of the collection of official peer-reviewed publications; extracting the plurality of first biomedical terms that are mentioned in the subset of official publications jointly with the target disease; and in accordance with an analysis of the official peer-reviewed publications, forming the first knowledge graph including connecting the target disease directly or indirectly with each of the plurality of first biomedical terms.

Clause 7. The method of any of clauses 1-6, wherein the general genetic variant information includes a second knowledge graph that couples the target disease and a plurality of second biomedical terms semantically to one another based on a private knowledge database;

Clause 8. The method of clause 7, wherein the private knowledge database includes information items collected from a group of subject experts including a set of experts of the target disease, the method further comprising: semantically analyzing a subset of information items; extracting the plurality of second biomedical terms that are mentioned in the subset of information items jointly with the target disease; and in accordance with a semantic analysis of the information items, forming the second knowledge graph including connecting the target disease directly or indirectly with each of the plurality of second biomedical terms.

Clause 9. The method of any of clauses 1-8, wherein the general genetic variant information includes genomic related information on annotated and predicted human genes, wherein the method further comprising: extracting, from a first database, the general gene information of the genomic related information that is associated with the plurality of subject genetic variants.

Clause 10. The method of any of clauses 1-9, wherein the general genetic variant information includes information about associations between human gene variants and phenotypes, the method further comprising: obtaining, from a second database, the associations between human gene variants and phenotypes, wherein the corresponding general genetic variants are prioritized based on their corresponding associations with a plurality of general phenotypes of the target disease.

Clause 11. The method of clause 10, obtaining the associations between human gene variants and phenotypes further comprises: providing a query to the second database, the query identifying the target disease; and in response to the query, extracting the information about the associations between human gene variants and phenotypes from the second database.

Clause 12. The method of any of clauses 1-4, wherein the general genetic variant information includes one or more of: a first knowledge graph that couples the target disease and a plurality of first biomedical terms semantically to one another based on a knowledge database; a second knowledge graph that couples the target disease and a plurality of second biomedical terms semantically to one another based on a private knowledge database; general gene information of a plurality of human genes which includes at least a subset of first genes that are associated with the plurality of subject genetic variants; and genetic variant information that identifies and prioritizes corresponding general genetic variants based on a plurality of general phenotypes of the target disease.

Clause 13. The method of any of clauses 1-12, further comprising:

Determining that the general genetic variant information is updated; in accordance with a determination that the general genetic variant information is updated, re-applying the biomimetic information model to update the set of target genetic variants.

Clause 14. The method of any of clauses 1-13, wherein the set of target genetic variants includes a first number of genetic variants associated with the target disease, and the subset of subject genetic variants includes a second number of genetic variants associated with the target disease, the first number is at least two orders smaller than the second number.

Clause 15. The method of any of clauses 1-14, wherein: each of the subset of subject genetic variants is detected in samples of a respective number of patients in the plurality of patients; the subject genetic variant information includes the respective number of patients corresponding to each variant of the subset of subject genetic variants; and the subset of subject genetic variants is ranked partially based on the respective number of patients corresponding to each of the subset of subject genetic variants.

Clause 16. The method of any of clauses 1-15, wherein the target disease includes endometriosis and endometriosis-related infertility, and the set of target genetic variants includes an outlier genetic variant corresponding to at least one of MUC 20, USP17L1, FAM66B, and DEFB109B.

Clause 17. The method of any of clauses 1-16, wherein the target disease includes Rheumatoid Arthritis, and the set of target genetic variant includes an outlier genetic variant corresponding to at least one of HIF1A, HLA-DOA, PTGER3, HIPK3, TGFBR3 and HIF1A-AS3.

Clause 18. The method of any of clauses 1-17, wherein the set of target genetic variants includes a first subset of target genetic variants that are directly linked to the target disease and a second subset of target genetic variants that are indirectly linked to the target disease.

Clause 19. The method of any of clauses 1-18, wherein a filter is applied to select the subset of subject genetic variants from the plurality of subject genetic variants based on one or more of: a confidence score, a population frequency of occurrence, a predicted deleterious level, and a mode of inheritance.

Clause 20. The method of any of clauses 1-19, further comprising: ranking the set of target genetic variants based on a correlation level with the plurality of subject phenotypes of the target disease; and in accordance with ranking, associating each of the set of target genetic variants with one of a plurality of predefined genetic significance levels.

Clause 21. The method of clause 20, wherein the plurality of predefined genetic significance levels includes pathogenic, likely pathogenic, benign, and likely benign.

Clause 22. The method of clause 1, wherein the information of the plurality of subject genetic variants includes information of an outlier genetic variant, the information of the outlier genetic variant is not included in the general genetic variant information, and the outlier genetic variant is preserved in the set of target genetic variants after the biomimetic information model is applied and applied to identify the set of target genetic variants.

Clause 23. The method of any of clauses 1-22, wherein the biomimetic information model includes a neural network or a set of predefined information processing rules.

Clause 24. The method of any of clauses 1-23, wherein applying the biomimetic information model further comprises: quantitatively determining a set of transcription factors, a set of translation factors, a plurality of subject factors, a plurality of genetic factors, and a plurality of protein factors associated with the subset of subject genetic variants; and generating a score for the subset of subject genetic variants using a weighted combination of the set of transcription factors, the set of translation factors, the plurality of subject factors, the plurality of genetic factors, and the plurality of protein factors; wherein the variant selection criterion requires that the set of target genetic variants be ranked among a predefined number of subject genetic variants having the highest score.

Clause 25. A computer system, comprising: one or more processors; and memory having instructions stored thereon, which when executed by the one or more processors cause the processors to perform operations for implementing a method in any of clauses 1-22.

Clause 26. A non-transitory computer-readable medium, having instructions stored thereon, which when executed by one or more processors of a server system cause the processors to perform operations of a method of any of clauses 1-22.

Clause 27. A method of identifying a subject having a high risk of endometriosis, endometrial cancer, or an endometrial form of ovarian cancer comprising obtaining a biological sample from a subject; deriving a genomic DNA sample from the biological sample; and determining the said subject is a carrier of at least one variant within at least one gene selected from the group consisting of MUC 20, USP17L1, FAM66B, and DEFB109B.

Clause 28. The method of clause 27, wherein the SNP within the gene MUC20 is at the location of rs10794288 or rs10902088.

Clause 29. The method of clause 27, further comprising administering one or more therapeutic agents compounds for the treatment of endometriosis, endometrial cancer, or an endometrial form of ovarian cancer.

Clause 30. The method of clause 27, wherein the determining the said subject is a carrier of at least one variant is performed by a genotyping assay, polymerase chain reaction (PCR), reverse transcription PCR, quantitative PCR, a microarray, DNA sequencing, and/or RNA sequencing.

Clause 31. A kit comprising: at least one probe to selectively hybridize to at least one nucleotide variant under high stringency conditions and amplifies the nucleotide variance sequence but does not amplify a corresponding wild-type sequence, wherein the nucleotide variance comprises at least one variant within at least one gene selected from the group consisting of MUC 20, USP17L1, FAM66B, and DEFB109B.

Clause 32. A method of identifying a subject having a high risk of rheumatoid arthritis comprising: (a) obtaining a biological sample from a subject; (b) deriving a genomic DNA sample from the biological sample; and (c) determining the said subject is a carrier of at least one variant within at least one gene selected from the group consisting of HIF1A, HLA-DOA, PTGER3, HIPK3, TGFBR3 and HIF1A-AS3.

Clause 33. The method of clause 32, wherein the gene/genetic locus of the at least one variant is shown as in Table 4.

Clause 34. The method of clause 32, further comprising administering one or more therapeutic agents compounds for the treatment of rheumatoid arthritis.

Clause 35. The method of Clause 32, wherein the determining the said subject is a carrier of at least one variant is performed by a genotyping assay, polymerase chain reaction (PCR), reverse transcription PCR, quantitative PCR, a microarray, DNA sequencing, and/or RNA sequencing.

Clause 36. A kit comprising: at least one probe to selectively hybridize to at least one nucleotide variant under high stringency conditions and amplifies the nucleotide variant sequence but does not amplify a corresponding wild-type sequence, wherein the nucleotide variance comprises at least one variant within at least one gene selected from the group consisting of HIF1A, HLA-DOA, PTGER3, HIPK3, TGFBR3 and HIF1A-AS3.

What is claimed is:

1. A computer-implemented method for genetic testing, comprising:
obtaining information of a plurality of subject genetic variants that are identified from a plurality of biological samples of a plurality of patients who are diagnosed with a target disease;
selecting a subset of subject genetic variants from the plurality of subject genetic variants based on a plurality of subject phenotypes of the target disease;
ranking the subset of subject genetic variants based on the plurality of subject phenotypes to generate subject genetic variant information;
obtaining subject medical information of the plurality of patients, including doctor-inputted description of the target disease collected from the plurality of patients;
obtaining general genetic variant information, independently of the plurality of patients;
applying an information model to process the subject genetic variant information, the subject medical information, and the general genetic variant information of the target disease; and
identifying a set of target genetic variants from the subset of subject genetic variants, the set of target genetic variants satisfying a variant selection criterion based upon genotype-phenotype correlations to the disease being studied.

2. The method of claim 1, further comprising:
assessing the set of target variants as therapeutic targets; and
determining one or more compounds of a drug configured to treat the target disease,
wherein the set of target variants includes a first target variant, and assessing the set of target variants further comprising one or more of:
gathering clinical information about the plurality of patients;
classifying the first target variant to one of a set of classes including pathogenic, likely pathogenic, uncertain significance, likely benign, and benign;
conducting functional study to assess an impact of the first target variant on a protein function or expression;
assessing a frequency of the first target variant in one or more populations;
determining whether the first target variant co-segregates with a predefined phenotype;
predicting a functional impact of the first target variant based on a variant location within a corresponding gene and an effect on a protein structure; and
identifying a pathway affected by the first target variant and contributing to one or more phenotypes of the target disease.

3. The method of claim 2, wherein the one or more compounds of the drug include small molecules, antibodies, gene therapies, an immunotherapy, a chemotherapy, a radiation therapy, a hormone therapy, a photodynamic therapy, a targeted therapy, polynucleotide, natural compound, immune modulator, bone marrow therapy, stem cell therapy, surgery therapy, induction therapy, maintenance therapy, or a combination thereof.

4. The method of claim 1, wherein the general genetic variant information includes a first knowledge graph that couples the target disease and a plurality of first biomedical terms semantically to one another based on a public knowledge database.

5. The method of claim 4, wherein the public knowledge database includes a collection of official peer-reviewed publications, the method further comprising:
semantically analyzing a subset of the collection of official peer-reviewed publications;
extracting the plurality of first biomedical terms that are mentioned in the subset of official publications jointly with the target disease; and
in accordance with an analysis of the official peer-reviewed publications, forming the first knowledge graph including connecting the target disease directly or indirectly with each of the plurality of first biomedical terms.

6. The method of claim 1, wherein the general genetic variant information includes a second knowledge graph that couples the target disease and a plurality of second biomedical terms semantically to one another based on a private knowledge database.

7. The method of claim 6, wherein the private knowledge database includes information items collected from a group of subject experts including a set of experts of the target disease, the method further comprising:
semantically analyzing a subset of information items;
extracting the plurality of second biomedical terms that are mentioned in the subset of information items jointly with the target disease; and
in accordance with a semantic analysis of the information items, forming the second knowledge graph including connecting the target disease directly or indirectly with each of the plurality of second biomedical terms.

8. The method of claim 1, wherein the general genetic variant information includes genomic related information on annotated and predicted human genes, wherein the method further comprising:
extracting, from a first database, the general gene information of the genomic related information that is associated with the plurality of subject genetic variants.

9. The method of claim 1, wherein the general genetic variant information includes information about associations between human gene variants and phenotypes, the method further comprising:
obtaining, from a second database, the associations between human gene variants and phenotypes, wherein the corresponding general genetic variants are prioritized based on their corresponding associations with a plurality of general phenotypes of the target disease.

10. The method of claim 9, obtaining the associations between human gene variants and phenotypes further comprises:
providing a query to the second database, the query identifying the target disease; and
in response to the query, extracting the information about the associations between human gene variants and phenotypes from the second database.

11. The method of claim 1, wherein the general genetic variant information includes one or more of:
a first knowledge graph that couples the target disease and a plurality of first biomedical terms semantically to one another based on a public knowledge database;
a second knowledge graph that couples the target disease and a plurality of second biomedical terms semantically to one another based on a private knowledge database;
general gene information of a plurality of human genes which includes at least a subset of first genes that are associated with the plurality of subject genetic variants; and
genetic variant information that identifies and prioritizes corresponding general genetic variants based on a plurality of general phenotypes of the target disease.

12. The method of claim 1, wherein the set of target genetic variants includes a first number of genetic variants associated with the target disease, and the subset of subject genetic variants includes a second number of genetic variants associated with the target disease, the first number is at least two orders smaller than the second number.

13. The method of any of claim 1, wherein:
each of the subset of subject genetic variants is detected in samples of a respective number of patients in the plurality of patients;
the subject genetic variant information includes the respective number of patients corresponding to each variant of the subset of subject genetic variants; and
the subset of subject genetic variants is ranked partially based on the respective number of patients corresponding to each of the subset of subject genetic variants.

14. The method of claim 1, wherein the target disease includes endometriosis and endometriosis-related infertility, and the set of target genetic variants includes an outlier genetic variant corresponding to at least one of MUC 20, USP17L1, FAM66B, and DEFB109B.

15. The method of claim 1, wherein the target disease includes Rheumatoid Arthritis, and the set of target genetic variant includes an outlier genetic variant corresponding to at least one of HIF1A, HLA-DOA, PTGER3, HIPK3, TGFBR3 and HIF1A-AS3.

16. A computer system, comprising:
one or more processors; and
memory having instructions stored thereon, which when executed by the one or more processors cause the processors to perform operations further comprising:
obtaining information of a plurality of subject genetic variants that are identified from a plurality of biological samples of a plurality of patients who are diagnosed with a target disease;
selecting a subset of subject genetic variants from the plurality of subject genetic variants based on a plurality of subject phenotypes of the target disease;
ranking the subset of subject genetic variants based on the plurality of subject phenotypes to generate subject genetic variant information;
obtaining subject medical information of the plurality of patients, including doctor-inputted description of the target disease collected from the plurality of patients;
obtaining general genetic variant information, independently of the plurality of patients;
applying an information model to process the subject genetic variant information, the subject medical information, and the general genetic variant information of the target disease; and
identifying a set of target genetic variants from the subset of subject genetic variants, the set of target genetic variants satisfying a variant selection criterion based upon genotype-phenotype correlations to the disease being studied.

17. The computer system of any of claim 16, wherein the set of target genetic variants includes a first subset of target genetic variants that are directly linked to the target disease and a second subset of target genetic variants that are indirectly linked to the target disease.

18. The computer system of any of claim 16, wherein a filter is applied to select the subset of subject genetic variants from the plurality of subject genetic variants based on one or more of: a confidence score, a population frequency of occurrence, a predicted deleterious level, and a mode of inheritance.

19. The computer system of claim 16, further comprising:
ranking the set of target genetic variants based on a correlation level with the plurality of subject phenotypes of the target disease; and
in accordance with ranking, associating each of the set of target genetic variants with one of a plurality of predefined genetic significance levels.

20. The computer system of claim 19, wherein the plurality of predefined genetic significance levels includes pathogenic, likely pathogenic, benign, and likely benign.

21. The computer system of claim 16, wherein the information of the plurality of subject genetic variants includes information of an outlier genetic variant, the information of the outlier genetic variant is not included in the general genetic variant information, and the outlier genetic variant is preserved in the set of target genetic variants after the information model is applied and applied to identify the set of target genetic variants.

22. A non-transitory computer-readable storage medium, having instructions stored thereon, which when executed by one or more processors of a server system cause the processors to perform operations comprising:
obtaining information of a plurality of subject genetic variants that are identified from a plurality of biological samples of a plurality of patients who are diagnosed with a target disease;
selecting a subset of subject genetic variants from the plurality of subject genetic variants based on a plurality of subject phenotypes of the target disease;
ranking the subset of subject genetic variants based on the plurality of subject phenotypes to generate subject genetic variant information;
obtaining subject medical information of the plurality of patients, including doctor-inputted description of the target disease collected from the plurality of patients;
obtaining general genetic variant information, independently of the plurality of patients;
applying an information model to process the subject genetic variant information, the subject medical information, and the general genetic variant information of the target disease; and
identifying a set of target genetic variants from the subset of subject genetic variants, the set of target genetic variants satisfying a variant selection criterion based upon genotype-phenotype correlations to the disease being studied.

23. The non-transitory computer-readable storage medium of claim 22, wherein the information model includes a neural network or a set of predefined information processing rules.

24. The non-transitory computer-readable storage medium of claim 22, wherein applying the information model further comprises:
quantitatively determining a set of transcription factors, a set of translation factors, a plurality of subject factors, a plurality of genetic factors, and a plurality of protein factors associated with the subset of subject genetic variants; and
generating a score for the subset of subject genetic variants using a weighted combination of the set of transcription factors, the set of translation factors, the plurality of subject factors, the plurality of genetic factors, and the plurality of protein factors;
wherein the variant selection criterion requires that the set of target genetic variants be ranked among a predefined number of subject genetic variants having the highest score.
